(19) United States
(12) Patent Application Publication
(10) Pub. No.: US 2008/0242661 A1
(43) Pub. Date: Oct. 2, 2008

(54) Novel compounds useful for the treatment of degenerative & inflammatory diseases

(76) Inventors: Graeme James Dykes, Mechelen (BE); Stephen Robert Fletcher, Mechelen (BE); Christel Jeanne Marie Menet, Mechelen (BE); Nuria Merayo Merayo, Mechelen (BE); Benoit Antoine Schmitt, Mechelen (BE)

Correspondence Address: KLAUBER & JACKSON, 411 HACKENSACK AVENUE, HACKENSACK, NJ, 07601, US

(21) Appl. No.: 12/001,209

(22) Filed: Dec. 10, 2007

Related U.S. Application Data
(60) Provisional application No. 60/869,413, filed on Dec. 11, 2006.

(51) Int. Cl. A61K 31/551; A61K 31/519; A61K 31/5377; A61P 29/00; A61P 19/02; C07D 487/04

(52) U.S. Cl. 514/218; 544/262; 514/262.1; 540/575; 544/118; 514/234.2

(57) ABSTRACT

The present invention relates to compounds that are inhibitors of PDE1A, a phosphodiesterase that is involved in the modulation of the degradation of cartilage, joint degeneration and diseases involving such degradation and/or inflammation.

### RELATED APPLICATIONS

The present application claims the benefit under 35 U.S.C. § 119 of U.S. Provisional Application Ser. No. 60/869,413, filed Dec. 11, 2006, the contents of which is hereby incorporated by reference in its entirety.

### BACKGROUND OF THE INVENTION

### Field of Invention

The present invention relates to compounds that are inhibitors of PDE1A, a phosphodiesterase that is involved in the modulation of the degradation of cartilage, joint degeneration and diseases involving such degradation and/or inflammation.

Cartilage is an avascular tissue of which chondrocytes are the main cellular component. The chondrocytes in normal articular cartilage occupy approximately 5% of the tissue volume, while the extra-cellular matrix makes up the remaining 95% of the tissue. The chondrocytes secrete the components of the matrix, mainly proteoglycans and collagens, which in turn supply the chondrocytes with an environment suitable for their survival under mechanical stress. In cartilage, collagen type II, together with the protein collagen type IX, is arranged in solid fibril-like structures, which provide cartilage with great mechanical strength. The proteoglycans can absorb water and are responsible for the resilient and shock absorbing properties of the cartilage.

One of the functional roles of cartilage in the joint is to allow bones to articulate on each other smoothly. Loss of articular cartilage, therefore, causes the bones to rub against each other leading to pain and loss of mobility. The degradation of cartilage can have various causes. In inflammatory arthritis, as in rheumatoid arthritis for example, cartilage degradation is caused by the secretion of proteases (e.g. collagenases) by inflamed tissues (the inflamed synovium for example). Cartilage degradation can also be the result of an injury of the cartilage, due to an accident or surgery, or exaggerated loading or ‘wear and tear’. The ability of cartilage tissue to regenerate after such insults is limited. Chondrocytes in injured cartilage often display reduced cartilage synthesizing (anabolic) activity and/or increased cartilage degrading (catabolic) activity.

The degeneration of cartilage is the hallmark of various diseases, among which rheumatoid arthritis and osteoarthritis are the most prominent.

Rheumatoid arthritis (RA) is a chronic joint degenerative disease, characterized by inflammation and destruction of the joint structures. When the disease is unchecked, it leads to substantial disability and pain due to loss of joint functionality and even premature death. The aim of an RA therapy, therefore, is not to slow down the disease but to attain remission in order to stop the joint destruction. Besides the severity of the disease outcome, the high prevalence of RA (˜0.8% of adults are affected worldwide) means a high socioeconomic impact. (For reviews on RA, we refer to Smolen and Steiner (2003); Lee and Weinblatt (2001); Choy and Panayi (2001); O'Dell (2004) and Firestein (2003)).

Osteoarthritis (also referred to as OA, or wear-and-tear arthritis) is the most common form of arthritis and is characterized by loss of articular cartilage, often associated with hypertrophy of the bone and pain. The disease mainly affects hands and weight-bearing joints such as knees, hips and spines. This process thins the cartilage. When the surface area has disappeared due to the thinning, a grade I osteoarthritis is reached; when the tangential surface area has disappeared, grade II osteoarthritis is reached. There are further levels of degeneration and destruction, which affect the deep and the calcified cartilage layers that border with the subchondral bone. For an extensive review on Osteoarthritis, refer to Wieland et al., 2005.

The clinical manifestations of the development of the osteoarthritis condition include: increased volume of the joint, pain, crepitation and functional disability that, lead to pain and reduced mobility of the joints. When disease further develops, pain at rest emerges. If the condition persists without correction and/or therapy, the joint is destroyed leading to disability. Replacement surgery with total prosthesis is then required.

Therapeutic methods for the correction of the articular cartilage lesions that appear during the osteoarthritic disease have been developed, but so far none of them have been able to mediate the regeneration of articular cartilage in situ and in vivo.

Osteoarthritis is difficult to treat. At present, no cure is available and treatment focuses on relieving pain and preventing the affected joint from becoming deformed. Common treatments include the use of non-steroidal anti-inflammatory drugs (NSAID's). Although the dietary supplements as chondroitin and glucosamine sulphate have been advocated as safe and effective options for the treatment of osteoarthritis, a recent clinical trial revealed that both treatments did not reduce pain associated to osteoarthritis. (Clegg et al., 2006). Taken together, no disease modifying osteoarthritic drugs are available.

In severe cases, joint replacement may be necessary. This is especially true for hips and knees. If a joint is extremely painful and cannot be replaced, it may be fused. This procedure stops the pain, but results in the permanent loss of joint function, making walking and bending difficult.

Another possible treatment is the transplantation of cultured autologous chondrocytes. Here chondral cellular material is taken from the patient, sent to a laboratory where it is expanded. The material is then implanted in the damaged tissues to cover the tissue's defects.

Another treatment includes the intra-articular instillation of Hylan G-F 20 (Synvisc, Hyalgan, Artz etc.), a substance that improves temporarily the rheology of the synovial fluid, producing an almost immediate sensation of free movement and a marked reduction of pain.

Other reported methods include application of tendinous, periosteal, fascial, muscular or perichondral grafts; implantation of fibrin or cultured chondrocytes; implantation of synthetic matrices, such as collagen, carbon fiber; administration of electromagnetic fields. All of these have reported minimal and incomplete effects, resulting in a poor quality tissue that can neither support the weighted load nor allow the restoration of an articular function with normal movement.

Stimulation of the anabolic processes, blocking catabolic processes, or a combination of these two, may result in stabilization of the cartilage, and perhaps even reversion of the damage, and therefore prevent further progression of the disease. Various triggers may stimulate anabolic stimulation of chondrocytes. Insulin-like growth factor-I (IGF-I) is the predominant anabolic growth factor in synovial fluid and stimulates the synthesis of both proteoglycans and collagen. It has also been shown that members of the bone morphogenetic protein (BMP) family, notably BMP2, BMP4, BMP6, and BMP7, and members of the human transforming growth factor-b (TGF-b) family can induce chondrocyte anabolic stimulation (Chubinskaya and Kuettner, 2003). A compound has recently been identified that induces anabolic stimulation of chondrocytes (U.S. Pat. No. 6,500,854; EP 1.391211). However, most of these compounds show severe side effects and, consequently, there is a strong need for compounds that stimulate chondrocyte differentiation without these side effects.

Adenosine 3′,5′-cyclic monophosphate (cyclic AMP or cAMP) and guanosine 3′,5′-cyclic monophosphate (cyclic GMP or cGMP) are key second messenger molecules in cells which are synthesized by guanylyl and adenylyl cyclases. These molecules, by playing a role as ‘relay’ on signal transduction pathways, are key in controlling normal and pathological cell responses. Cyclic nucleotide phosphodiesterases (PDE's) are enzymes that hydrolyse cyclic nucleotides and thereby control the cellular levels of these second messenger molecules. Because of their key role in cellular signaling, PDE's are considered new therapeutic targets. Inhibition of PDE4 and PDE5 are accepted approaches for the treatment of asthma/chronic obstructive pulmonary disease and erectile dysfunction, respectively. As such, pharmaceutical industry has recently deployed a lot of efforts to develop PDE4 inhibitors (e.g. Cilomilast) and PDE5 inhibitors (e.g. sildenafil), some of which are marketed.

The diversity of the PDE family of enzymes (11 gene families (PDE1-PDE11) encoding more than 20 different PDE genes) allows a refined control over a variety of cellular processes. For an extensive review on PDE's, we refer to Lugnier, 2006. PDE's classically contain a catalytic domain, which is well conserved among different PDEs. In addition, PDE's contain regulatory domains. The activity of enzymes of the PDE1 subfamily, for example, is regulated by Ca²⁺ and calmodulin as well as by phosphorylation. As such, the PDE1 enzymes are involved in the complex interaction between the Ca²⁺ and cyclic nucleotide second messenger systems. Another feature of PDE1 enzymes is their dual substrate specificity as they have the capacity to hydrolyse both cAMP and cGMP (Zhang et al., 2004).

The generation of transgenic animals represents the best tool for the understanding of the specific physiological role of individual PDEs. In the PDE1 subfamily, a PDE1B knockout mouse has been generated and characterized. PDE1B(−/−) mice showed exaggerated hyperactivity after acute D-methamphetamine administration. PDE1B(−/−) and PDE1B(+/−) mice demonstrated spatial-learning deficits. These results indicate that enhancement of cyclic nucleotide signaling by inactivation of PDE1B-mediated cyclic nucleotide hydrolysis plays a significant role in the central nervous system, especially on the dopaminergic function (Reed et al., 2002). Less is known about the physiological role of the other members of the PDE1 superfamily. A role for PDE1 enzymes (PDE1C in particular) in vascular tone (e.g.; in pulmonary hypertension) has been suggested (Murray et al., 2006). For an extensive review on PDE1 enzymes, we refer to Kakkar et al., 1999 and Goraya and Cooper, 2005.

Several data point to a role of PDEs in chondrocyte biology. First, PDE4 and PDE1 were identified as major PDE activities in chondrocytes (Tenor et al., 2002). The involvement of PDEs in cartilage catabolic events was further evidenced as follows. The IL1 cytokine is responsible for cartilage catabolism by reducing the expression of matrix components, by inducing the expression of collagenases and inducible nitric oxide synthase (iNOS), which mediates the production of nitric oxide (NO). This event appears dependent on PDE activity, as IBMX, PDE5 inhibitor and PDE4 inhibitor treatment of chondrocytes reduced the induction of iNOS expression by ELI (Geng et al., 1998, Tenor et al., 2002). The ability of PDE inhibitors to reduce iNOS expression appeared dependent on autocrine PGE2 production by the chondrocytes. Taken together, these data suggest a role for PDEs in cartilage catabolic events.

The current therapies are not satisfactory and therefore there remains a need to identify further compounds that may be of use in the treatment of degenerative joint diseases, e.g. osteoarthritis, rheumatoid arthritis and osteoporosis. The present invention therefore provides compounds, methods for their manufacture and a pharmaceutical comprising a compound of the invention together with a suitable pharmaceutical carrier. The present invention also provides for the use of a compound of the invention in the preparation of a medicament for the treatment of degenerative joint diseases.

### SUMMARY OF THE INVENTION

The present invention is based on the discovery that inhibitors of PDE1A are useful for the treatment of diseases involving cartilage degradation, joint degradation and/or inflammation, for example osteoarthritis. The present invention also provides methods for the production of these compounds, pharmaceutical compositions comprising these compounds and methods for treating diseases involving cartilage degradation, joint degradation and/or inflammation by administering a compound of the invention.

Accordingly, the present invention relates to compounds having anti-inflammatory properties, according to formula (I):

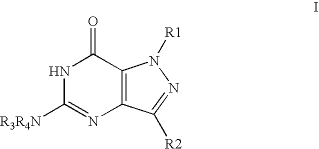

In which:
R¹ represents a group selected from H, C₁-C₆ alkyl, C₂-C₆ alkenyl, C₂-C₆ alkynyl, cycloalkyl optionally substituted with one or more of OH, CF₃, CN, halogen, —CONH₂ or C₁-C₆ alkyl;
R² represents a group selected from H, C₁-C₆ alkyl, C₂-C₆ alkenyl, C₂-C₆ alkynyl, cycloalkyl optionally substituted with one or more of OH, CF₃, CN, halogen, —CONH₂, or C₁-C₆ alkyl;
R³ represents (i) alkyl, (ii) substituted or unsubstituted aryl or substituted or unsubstituted aralkyl, (iii) substituted or unsubstituted heteroaryl or substituted or unsubstituted heteroarylalkyl;
R⁴ represents H or alkyl;
or R³ and R⁴ together with the N to which they are attached may form
   (i) heterocycloalkyl unsubstituted or substituted with alkyl, aralkyl, halo substituted aralkyl, heteroarylalkyl or halo substituted heteroarylalkyl; or
   (ii) 1,2,3,4-tetrahydroisoquinoline;
      or a pharmaceutically acceptable salt, hydrate, solvate or prodrug thereof or isotopic variants thereof, stereoisomers or tautomers thereof.

In one embodiment, the present invention relates to compounds having anti-inflammatory properties, according to formula (I), above, and wherein:
R¹ represents a group selected from H, C₁-C₆ alkyl, C₂-C₆ alkenyl, C₂-C₆ alkynyl, cycloalkyl optionally substituted with one or more of OH, CF₃, CN, halogen, —CONH₂, or C₁-C₆ alkyl;
R² represents a group selected from H, C₁-C₆ alkyl, C₂-C₆ alkenyl, C₂-C₆ alkynyl, cycloalkyl optionally substituted with one or more of OH, CF₃, CN, halogen, —CONH₂, or C₁-C₆ alkyl;
R³ represents (i) C₁-C₆ alkyl, (ii) (CH₂)m-aryl, where the aryl may optionally be substituted by one or more of halogen, CF₃, OCH₃, heteroaryl, CH₂-heterocycloalkyl containing 2 or more heteroatoms, N-linked heterocycloalkyl optionally substituted with one or more of C₁-C₄ alkyl, CF₃, halogen, or heteroaryl, (iii) (CH₂)ₙ-heteroaryl, where the heteroaryl may optionally be substituted by one or more aryl or two or more C₁-C₄ alkyl, or (iv) aryl optionally substituted by one or more halogen;
R⁴ represents H or C₁-C₆ alkyl;
or R³ and R⁴ together with the N to which they are attached may form (i) a 4-7 membered nitrogen-containing heterocycloalkyl, optionally substituted with one or more of (CH₂)n-heteroaryl or (CH₂)n-aryl optionally substituted with one or more halogen, or (ii) 1,2,3,4-tetrahydroisoquinolinyl;
each “m” independently represents 0, 1 or 2;
each “n” independently represents 0 or 1;
   or a pharmaceutically acceptable salt, hydrate, solvate or prodrug thereof or isotopic variants thereof, stereoisomers or tautomers thereof.

More particularly, the present invention relates to compounds according to formulae Ia, Ib, or Ic:

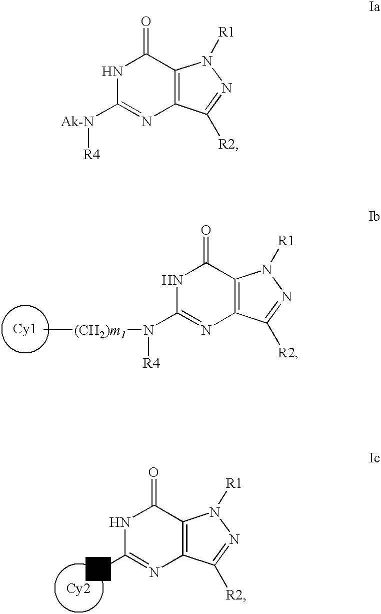

wherein:
Ak is C₁-C₆ alkyl;
Cy1 is substituted or unsubstituted aryl or substituted or unsubstituted heteroaryl;
Cy2 is heterocycloalkyl unsubstituted or substituted with C₁-C₆ alkyl, aralkyl, halo substituted aralkyl, heteroarylalkyl or halo substituted heteroarylalkyl; or Cy2 is 1,2,3,4-tetrahydroisoquinoline;
R¹ is H, C₁-C₆ alkyl, C₂-C₆ alkenyl, C₂-C₆ alkynyl, cycloalkyl optionally substituted with one or more of OH, CF₃, CN, halogen, —CONH₂ or C₁-C₆ alkyl;
R² is H, C₁-C₆ alkyl, C₂-C₆ alkenyl, C₂-C₆ alkynyl, cycloalkyl optionally substituted with one or more of OH, CF₃, CN, halogen, —CONH₂, or C₁-C₆ alkyl;
R⁴ is H, or C₁-C₆ alkyl; and
m1 is 0, 1, or 2; provided that when HetAr group is joined to —(CH₂)m1- via a N atom of HetAr group then m1 is 2;
   or a pharmaceutically acceptable salt, hydrate, solvate or prodrug thereof or isotopic variants thereof, stereoisomers or tautomers thereof.

In one embodiment, with respect to compounds of formulae Ia-Ic, R¹ is C₁-C₆ alkyl. In another embodiment R¹ is Me.

In one embodiment, with respect to compounds of formulae Ia-Ic, R² is C₁-C₆ alkyl or cycloalkyl. In another embodiment R² is C₁-C₆ alkyl.

In one embodiment, with respect to compounds of formulae Ia-Ib, R⁴ is C₁-C₆ alkyl.

Another aspect of this invention relates to the use of the present compound in a therapeutic method, a pharmaceutical composition, and the manufacture of such composition, useful for the treatment of a disease involving inflammation, and in particular, a disease characteristic of abnormal PDE1A activity. This invention also relates to processes for the preparation of the present compounds.

Other objects and advantages will become apparent to those skilled in the art from a consideration of the ensuing detailed description with reference to the following illustrative drawings.

### BRIEF DESCRIPTION OF THE DRAWINGS

FIG. 1. Shows the mechanism of the primary screening assay using the cAMP dynamic htrf kit from Cisbio.

### DETAILED DESCRIPTION

### Definitions

The following terms are intended to have the meanings presented therewith below and are useful in understanding the description and intended scope of the present invention.

When describing the compounds, pharmaceutical compositions containing such compounds and methods of using such compounds and compositions, the following terms have the following meanings unless otherwise indicated. It should be further understood that the terms “groups” and “radicals” can be considered interchangeable when used herein.

The articles “a” and “an” may be used herein to refer to one or to more than one (i.e. at least one) of the grammatical objects of the article. By way of example “an analogue” means one analogue or more than one analogue.

‘Alkoxy’ means alkyl-O—. Exemplary alkoxy includes methoxy, ethoxy, n-propoxy, i-propoxy, n-butoxy, and heptoxy. Particular alkoxy groups are lower alkoxy, i.e. with between 1 and 6 carbon atoms.

‘Alkyl’ means straight or branched aliphatic hydrocarbon having 1 to 20 carbon atoms. In particular, alkyl has 1 to 12 carbon atoms. The term ‘lower alkyl’ means 1 to 6 carbon atoms in a linear alkyl chain that may be straight or branched. Further particular groups are groups such as methyl (Me), ethyl (Et), propyl (Pr) and butyl (Bu). Branched means that one or more lower alkyl groups such as methyl, ethyl or propyl is attached to a linear alkyl chain. The term alkyl includes both branched and straight chain groups, exemplary straight chain groups include ethyl, n-propyl (n-Pr), n-butyl (n-Bu) as listed above, exemplary branched chain groups include isopropyl (i-Pr), tert-butyl (t-Bu) isoamyl.

‘Alkyl amino’ means alkyl-NH—. Particular alkyl amino is (C₁-C₆)-alkyl amino. Exemplary alkyl amino includes methylamino and ethylamino.

‘Amino lower alkanoyl’ means NH₂—R—CO—, where R is lower alkylene. Particular groups include aminoethanoyl and aminoacetyl.

‘Aralkyl’ or ‘arylalkyl’ refers to a radical in which an aryl group is substituted for a hydrogen atom of an alkyl group.

‘Acyl’ refers to a radical —C(O)R²⁰, where R²⁰ is hydrogen, alkyl, cycloalkyl, cycloheteroalkyl, aryl, arylalkyl, heteroalkyl, heteroaryl, heteroarylalkyl as defined herein. Representative examples include, but are not limited to, formyl, acetyl, cyclohexylcarbonyl, cyclohexylmethylcarbonyl, benzoyl, benzylcarbonyl and the like.

‘Aryl’ refers to a monovalent aromatic hydrocarbon group derived by the removal of one hydrogen atom from a single carbon atom of a parent aromatic ring system. Aryl groups may be monocyclic or a bicyclic fused-ring structure where at least one of the rings is an aromatic ring structure that particularly contains 6 carbons. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta 2,4 diene, pentacene, pentalene, pentaphene, perylene, phenalene, phenanthrene, picene, pleiadene, pyrene, pyranthrene, rubicene, triphenylene, trinaphthalene and the like. Particularly, an aryl group comprises from 6 to 14 carbon atoms. Particularly, the aryl group may contain 6 carbon atoms, exemplary aryl groups include phenyl and indan-1-one.

‘Substituted Aryl’ includes those groups recited in the definition of “substituted” herein, and particularly refers to an aryl group that may optionally be substituted with 1 or more substituents, for instance from 1 to 5 substituents, particularly 1 to 3 substituents, selected from the group consisting of acyl, acylamino, acyloxy, alkenyl, substituted alkenyl, alkoxy, substituted alkoxy, alkoxycarbonyl, alkyl, substituted alkyl, alkynyl, substituted alkynyl, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, nitro, thioalkoxy, substituted thioalkoxy, thioaryloxy, thiol, alkyl-S(O)—, aryl-S(O)—, alkyl-S(O)₂— and aryl-S(O)₂—.

‘Bicycloaryl’ refers to a monovalent aromatic hydrocarbon group derived by the removal of one hydrogen atom from a single carbon atom of a parent bicycloaromatic ring system. Typical bicycloaryl groups include, but are not limited to, groups derived from indane, indene, naphthalene, tetrahydronaphthalene, and the like. Particularly, an aryl group comprises from 8 to 11 carbon atoms.

‘Carbamoyl’ refers to the radical —C(O)N(R⁴²)₂ where each R⁴² group is independently hydrogen, alkyl, cycloalkyl or aryl, as defined herein, which may be optionally substituted as defined herein. In a specific embodiment, the term “carbamoyl” refers to —C(O)—NH₂. In an alternative embodiment ‘carbamoyl lower alkyl’ means the radical NH₂CO-lower alkyl-. Particular carbamoyl lower alkyl groups include carbamoylethyl and carbamoylmethyl.

‘Carboxy lower alkyl ester’ means a lower alkyl ester of a carboxy radical, —COO— group.

‘Compounds of the present invention’, and equivalent expressions, are meant to embrace the compounds as hereinbefore described, in particular compounds according to formula I and/or formulae Ia-Ic, or any of the formulae herein described, which expression includes the prodrugs, the pharmaceutically acceptable salts, and the solvates, e.g., hydrates, where the context so permits. Similarly, reference to intermediates, whether or not they themselves are claimed, is meant to embrace their salts, and solvates, where the context so permits.

‘Cycloalkylalkyl’ refers to a radical in which a cycloalkyl group is substituted for a hydrogen atom of an alkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

‘Heterocycloalkylalkyl’ refers to a radical in which a heterocycloalkyl group is substituted for a hydrogen atom of an alkyl group. Typical heterocycloalkylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

‘Expression’ means endogenous or exogenous expression.

‘Halo’ or ‘halogen’ means fluoro (F), chloro (Cl), bromo (Br), or iodo (I).

‘Hydrogen’ means in the context of a substituent that —H is present at the compound position and also includes its isotope, deuterium.

‘Lower alkanoyl amino’ means an amino group with an organic functional group R—CO—, where R represents a lower alkyl group.

‘Lower alkoxy’ means 1 to 6 carbon atoms in a linear alkyl chain that may be straight or branched, and that is bonded by an oxygen atom.

‘Lower alkyl sulphonamide’ refers to a lower alkyl amide of sulphonamide of the formula —SO₂NR*R*, where R* is hydrogen or lower alkyl, and at least one R* is lower alkyl.

‘Sulphonamide’ refers to a group of compounds containing the chemical group —SO₂NH₂.

‘Cycloalkyl’ refers to cyclic hydrocarbyl groups having from 3 to 10 carbon atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, and multiple ring structures such as adamantanyl, and the like. Particular cycloalkyl groups have between 4 and 7 carbon ring members for example cyclopropyl, cyclobutyl, cyclopentyl, cyclohexyl and cycloheptyl.

‘Substituted cycloalkyl’ includes those groups recited in the definition of ‘substituted’ herein, and particularly refers to a cycloalkyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

‘Substituted’ refers to a group in which one or more hydrogen atoms are each independently replaced with the same or different substituent(s). Typical substituents include, but are not limited to, —X, —R⁴⁶, —O, ═O, —OR⁴⁶, —SR⁴⁶, —S, ═S, NR⁴⁶R⁴⁷, ═NR⁴⁶, —CX₃, —CF₃, —CN, —OCN, —SCN, —NO, —NO₂, ═N₂, —N₃, —S(O)₂O, S(O)₂OH, —S(O)₂R₄₆, —OS(O₂)O—, —OS(O)₂R⁴⁶, —P(O)(O—)₂, —P(O)(OR⁴⁶)(O—), OP(O)(OR⁴⁶)(OR⁴⁷), —C(O)R⁴⁶, —C(S)R⁴⁶, —C(O)OR⁴⁶, —C(O)NR⁴⁶R⁴⁷, —C(O)O, C(S)OR⁴⁶, N⁴⁸C(O)NR⁴⁶R⁴⁷, —NR⁴⁸C(S)NR⁴⁶R⁴⁷, NR⁴⁹C(NR⁴⁸)NR⁴⁶R⁴⁷ and C(NR⁴⁸)NR⁴⁶R⁴⁷, where each X is independently a halogen; each R⁴⁶, R⁴⁷, R⁴⁸ and R⁴⁹ are independently hydrogen, alkyl, substituted alkyl, aryl, substituted aryl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR⁵⁰R⁵¹, —C(O)R⁵⁰ or S(O)₂R⁵⁰ or optionally R⁵⁰ and R⁵¹ together with the atom to which they are both attached form a cycloheteroalkyl or substituted cycloheteroalkyl ring; and R⁵⁰ and R⁵¹ are independently hydrogen, alkyl, substituted alkyl, aryl, substituted alkyl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl or substituted heteroarylalkyl.

Examples of representative substituted aryls include the following

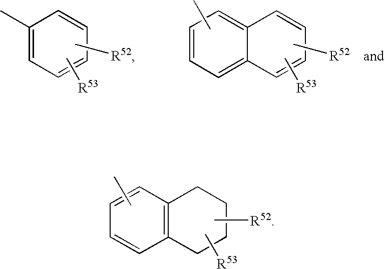

In these formulae one of R⁵² and R⁵³ may be hydrogen and at least one of R⁵² and R⁵³ is each independently selected from alkyl, alkenyl, alkynyl, cycloheteroalkyl, alkanoyl, alkoxy, aryloxy, heteroaryloxy, alkylamino, arylamino, heteroarylamino, NR⁵⁴COR⁵⁵, NR⁵⁴SOR⁵⁵, NR⁵⁴SO2R⁵⁷, COO-alkyl, COO-aryl, CONR⁵⁴R⁵⁵, CONR⁵⁴OR⁵⁵, NR⁵⁴R⁵⁵, SO2NR⁵⁴R⁵⁵, S-alkyl, S-alkyl, SO-alkyl, SO₂-alkyl, S-aryl, SO-aryl, SO₂-aryl; or R⁵² and R⁵³ may be joined to form a cyclic ring (saturated or unsaturated) from 5 to 8 atoms, optionally containing one or more heteroatoms selected from the group N, O or S. R⁵⁴, R⁵⁵, and R⁵⁶ are independently hydrogen, alkyl, alkenyl, alkynyl, perfluoroalkyl, cycloalkyl, cycloheteroalkyl, aryl, substituted aryl, heteroaryl, substituted or hetero alkyl or the like.

‘Hetero’ when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloalkyl, e.g. heterocycloalkyl, aryl, e.g. heteroaryl, cycloalkenyl, heterocycloalkenyl, and the like having from 1 to 5, and especially from 1 to 3 heteroatoms.

‘Heteroaryl’ refers to a monovalent heteroaromatic group derived by the removal of one hydrogen atom from a single atom of a parent heteroaromatic ring system. The heteroaryl group may be a monocyclic group (in which case it will typically be a 5 to 7, more typically a 5 or 6 membered ring), alternatively the heteroaryl group may be a bicycloheteroaryl group in particular a fused ring system comprising 2 fused 5-membered rings, a fused 5 and 6 membered ring or two fused 6 membered rings, where the heteroaryl group comprises fused rings at least one of said rings should contain a heteroatom and at least one said rings should be aromatic (both requirements may or may not be fulfilled in the same ring). The heteroaryl group can be, for example, a five membered or six membered monocyclic ring which may contain up to about four heteroatoms typically selected from nitrogen, sulphur and oxygen. Typically the heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Particularly, the heteroaryl group is between 5-15 membered heteroaryl, with 5-10 membered heteroaryl being particular groups. Particular heteroaryl groups are those derived from thiophene, pyrrole, benzothiophene, benzofuran, indole, pyridine, quinoline, imidazole, oxazole and pyrazine. Particularly, examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups. Particularly, examples of six membered heteroaryl groups include but are not limited to pyridine, pyrazine, pyridazine, pyrimidine and triazine.

Examples of representative heteroaryls include the following:

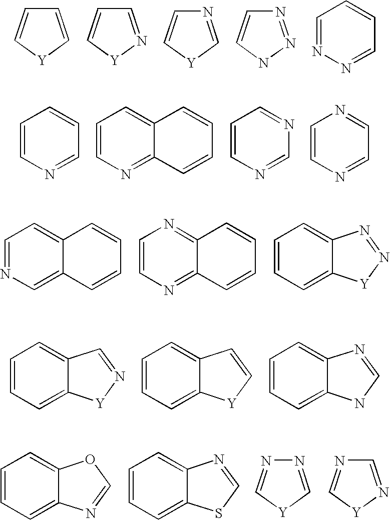

wherein each Y is selected from carbonyl, N, NR⁵⁸, O, and S; and R⁵⁸ is independently hydrogen, alkyl, cycloalkyl, cycloheteroalkyl, aryl, heteroaryl, heteroalkyl or the like.

‘Bicycloheteroaryl’ refers to a monovalent bicycloheteroaromatic group derived by the removal of one hydrogen atom from a single atom of a parent bicycloheteroaromatic ring system. Typical bicycloheteroaryl groups include, but are not limited to, groups derived from benzofuran, benzimidazole, benzindazole, benzdioxane, chromene, chromane, cinnoline, phthalazine, indole, indoline, indolizine, isobenzofuran, isochromene, isoindole, isoindoline, isoquinoline, benzothiazole, benzoxazole, naphthyridine, benzoxadiazole, pteridine, purine, benzopyran, benzpyrazine, pyridopyrimidine, quinazoline, quinoline, quinolizine, quinoxaline, benzomorphan, tetrahydroisoquinoline, tetrahydroquinoline, and the like. Particularly, the bicycloheteroaryl group is between 9-11 membered bicycloheteroaryl, with 5-10 membered heteroaryl being particular groups. Particular bicycloheteroaryl groups are those derived from benzothiophene, benzofuran, benzothiazole, indole, quinoline, isoquinoline, benzimidazole, benzoxazole, benzo[1,3]dioxalyl and benzodioxane.

‘Heterocycloalkyl’ refers to a stable heterocyclic non-aromatic ring and fused rings containing one or more heteroatoms independently selected from N, O and S. A fused heterocyclic ring system may include carbocyclic rings and need only include one heterocyclic ring. Examples of heterocyclic rings include, but are not limited to, piperazinyl, homopiperazinyl, piperidinyl and morpholinyl, and are shown in the following illustrative examples:

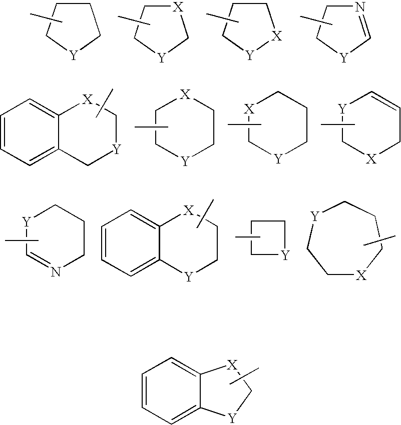

wherein each X is selected from CR⁵⁸₂, NR⁵⁸, O and S; and each Y is selected from NR⁵⁸, O and S; and R⁵⁸ is independently hydrogen, alkyl, cycloalkyl, cycloheteroalkyl, aryl, heteroaryl, heteroalkyl or the like. These cycloheteroalkyl rings may be optionally substituted with one or more groups selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—. Substituting groups include carbonyl or thiocarbonyl which provide, for example, lactam and urea derivatives.

“Nitrogen-Containing Heterocycloalkyl” group means a 4 to 7 membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

Examples of representative aryl having hetero atoms containing substitution include the following:

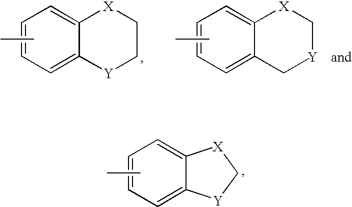

wherein each X is selected from CR⁵⁸₂, NR⁵⁸, O and S; and each Y is selected from carbonyl, NR⁵⁸, O and S; and R⁵⁸ is independently hydrogen, alkyl, cycloalkyl, cycloheteroalkyl, aryl, heteroaryl, heteroalkyl or the like.

One having ordinary skill in the art of organic synthesis will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

‘Sulfonyl’ refers to the group —SO₂R⁶³. In particular embodiments, R⁶³ is selected from H, lower alkyl, alkyl, aryl and heteroaryl.

‘Pharmaceutically acceptable’ means approved by a regulatory agency of the Federal or a state government or listed in the U.S. Pharmacopoeia or other generally recognized pharmacopoeia for use in animals, and more particularly in humans.

‘Pharmaceutically acceptable vehicle’ refers to a diluent, adjuvant, excipient or carrier with which a compound of the invention is administered.

‘Pharmaceutically acceptable salt’ refers to the non-toxic, inorganic and organic acid addition salts, and base addition salts, of compounds of the present invention, in particular they are pharmaceutically acceptable and possess the desired pharmacological activity of the parent compound. These salts can be prepared in situ during the final isolation and purification of compounds useful in the present invention. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term “pharmaceutically acceptable cation” refers to a non toxic, acceptable cationic counter-ion of an acidic functional group. Such cations are exemplified by sodium, potassium, calcium, magnesium, ammonium, tetraalkylammonium cations, and the like.

‘Solvate’ means a physical association of a compound useful in this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. Conventional solvents include water, ethanol, acetic acid and the like, therefore, representative solvates include hydrates, ethanolates and methanolates.

‘Prodrugs’ refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particular prodrugs. In some cases it is desirable to prepare double ester type prodrugs such as (acyloxy)alkyl esters or ((alkoxycarbonyl)oxy)alkylesters. Particularly the C₁ to C₈ alkyl, C₂-C₈ alkenyl, aryl, C₇-C₁₂ substituted aryl, and C₇-C₁₂ arylalkyl esters of the compounds of the invention.

‘Isotopic variant’ refers to a compound that contains unnatural proportions of isotopes at one or more of the atoms that constitute such compound. For example, an “isotopic variant” of a compound can contain one or more non-radioactive isotopes, such as for example, deuterium (²H or D), carbon 13 (¹³C), nitrogen-15 (¹⁵N), or the like. It will be understood that, in a compound where such isotopic substitution is made, the following atoms, where present, may vary, so that for example, any hydrogen may be 2H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy. All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

‘Tautomers’ refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

‘Subject’ includes humans. The terms ‘human’, ‘patient’ and ‘subject’ are used interchangeably herein.

‘Prophylaxis’ means a measure taken for the prevention of a disease.

‘Preventing’ or ‘prevention’ refers to a reduction in risk of acquiring a disease or disorder (i.e., causing at least one of the clinical symptoms of the disease not to develop in a subject that may be exposed to or predisposed to the disease but does not yet experience or display symptoms of the disease).

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

‘Therapeutically effective amount’ means that amount of a drug or pharmaceutical agent that will elicit the biological or medical response of a subject that is being sought by a medical doctor or other clinician. The “therapeutically effective amount” can vary depending on the compound, the disease and its severity, and the age, weight, etc., of the subject to be treated.

### THE COMPOUNDS

The compounds of the present invention may be described generally as pyrazolo[4,3-d]pyrimidin-7(6H)-ones substituted in the 5-position.

Accordingly, the present invention relates to compounds having anti-inflammatory properties, according to formula (I):

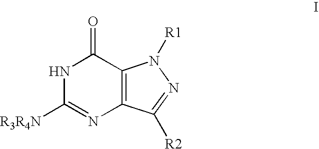

In which:
R¹ represents a group selected from H, C₁-C₆ alkyl, C₂-C₆ alkenyl, C₂-C₆ alkynyl, cycloalkyl optionally substituted with one or more of OH, CF₃, CN, halogen, —CONH₂, or C₁-C₆ alkyl;
R² represents a group selected from H, C₁-C₆ alkyl, C₂-C₆ alkenyl, C₂-C₆ alkynyl, cycloalkyl optionally substituted with one or more of OH, CF₃, CN, halogen, —CONH₂, or C₁-C₆ alkyl;
R³ represents (i) alkyl, (ii) substituted or unsubstituted aryl or substituted or unsubstituted aralkyl, (iii) substituted or unsubstituted heteroaryl or substituted or unsubstituted heteroarylalkyl;
R⁴ represents H or alkyl;
or R³ and R⁴ together with the N to which they are attached may form
   (iii) heterocycloalkyl unsubstituted or substituted with alkyl, aralkyl, halo substituted aralkyl, heteroarylalkyl or halo substituted heteroarylalkyl; or
   (iv) 1,2,3,4-tetrahydroisoquinoline;
      or a pharmaceutically acceptable salt, hydrate, solvate or prodrug thereof or isotopic variants thereof, stereoisomers or tautomers thereof.

In one embodiment, the present invention relates to compounds having anti-inflammatory properties, according to formula (I), above, and wherein:
R¹ represents a group selected from H, C₁-C₆ alkyl, C₂-C₆ alkenyl, C₂-C₆ alkynyl, cycloalkyl optionally substituted with one or more of OH, CF₃, CN, halogen, —CONH₂, or C₁-C₆ alkyl;
R² represents a group selected from H, C₁-C₆ alkyl, C₂-C₆ alkenyl, C₂-C₆ alkynyl, cycloalkyl optionally substituted with one or more of OH, CF₃, CN, halogen, —CONH₂, or C₁-C₆ alkyl;
R³ represents (i) C₁-C₆ alkyl, (ii) (CH₂)m-aryl, where the aryl may optionally be substituted by one or more of halogen, CF₃, OCH₃, heteroaryl, CH₂-heterocycloalkyl containing 2 or more heteroatoms, N-linked heterocycloalkyl optionally substituted with one or more of C₁-C₄ alkyl, CF₃, halogen, or heteroaryl, (iii) (CH₂)ₙ-heteroaryl, where the heteroaryl may optionally be substituted by one or more aryl or two or more C₁-C₄ alkyl, or (iv) aryl optionally substituted by one or more halogen;
R⁴ represents H or C₁-C₆ alkyl;
or R³ and R⁴ together with the N to which they are attached may form (i) a 4-7 membered nitrogen-containing heterocycloalkyl, optionally substituted with one or more of (CH₂)n-heteroaryl or (CH₂)n-aryl optionally substituted with one or more halogen, or (ii) 1,2,3,4-tetrahydroisoquinolinyl;
each “m” independently represents 0, 1 or 2;
each “n” independently represents 0 or 1;
   or a pharmaceutically acceptable salt, hydrate, solvate or prodrug thereof or isotopic variants thereof, stereoisomers or tautomers thereof.

More particularly, the present invention relates to compounds according to formulae Ia, Ib, or Ic:

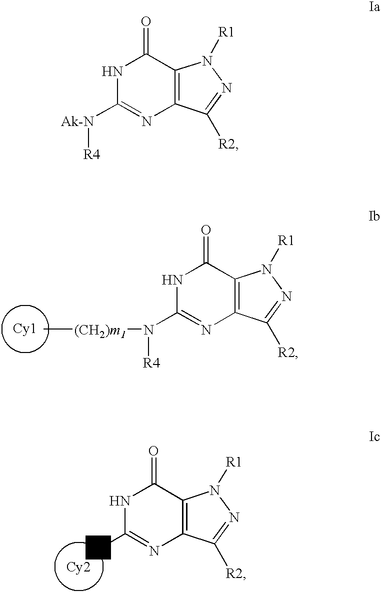

wherein:
Ak is C₁-C₆ alkyl;
Cy1 is substituted or unsubstituted aryl or substituted or unsubstituted heteroaryl;
Cy2 is heterocycloalkyl unsubstituted or substituted with C₁-C₆ alkyl, aralkyl, halo substituted aralkyl, heteroarylalkyl or halo substituted heteroarylalkyl; or Cy2 is 1,2,3,4-tetrahydroisoquinoline;
R¹ is H, C₁-C₆ alkyl, C₂-C₆ alkenyl, C₂-C₆ alkynyl, cycloalkyl optionally substituted with one or more of OH, CF₃, CN, halogen, —CONH₂, or C₁-C₆ alkyl;
R² is H, C₁-C₆ alkyl, C₂-C₆ alkenyl, C₂-C₆ alkynyl, cycloalkyl optionally substituted with one or more of OH, CF₃, CN, halogen, —CONH₂, or C₁-C₆ alkyl;
R⁴ is H, or C₁-C₆ alkyl; and
m1 is 0, 1, or 2; provided that when HetAr group is joined to —(CH₂)m1- via a N atom of HetAr group then m1 is 2;
   or a pharmaceutically acceptable salt, hydrate, solvate or prodrug thereof or isotopic variants thereof, stereoisomers or tautomers thereof.

In one embodiment, with respect to compounds of formulae Ia-Ic, R¹ is C₁-C₆ alkyl. In another embodiment R¹ is Me.

In one embodiment, with respect to compounds of formulae Ia-Ic, R² is C₁-C₆ alkyl or cycloalkyl. In another embodiment R² is C₁-C₆ alkyl.

In one embodiment, with respect to compounds of formulae Ia-Ib, R⁴ is alkyl.

In one embodiment, with respect to compounds of formulae Ia-Ic, the compound is according to formula Ia:

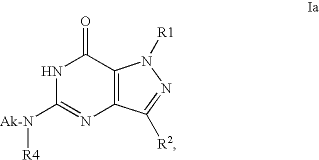

wherein Ak, R¹, R² and R⁴ are as described above.

In one embodiment, with respect to compounds of formulae Ia-Ic, the compound is according to formula Ib:

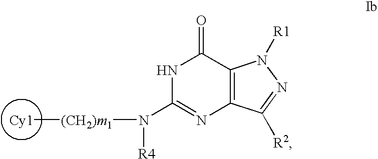

wherein Cy1, R¹, R², R⁴ and ml are as described above.

In one embodiment, with respect to compounds of formulae Ia-Ic, the compound is according to formula Ic:

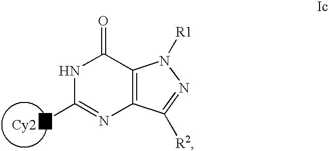

wherein Cy2, R¹, and R² as described above.

In one embodiment, with respect to compounds of formulae Ia-Ic, R¹ is H or C₁-C₆ alkyl.

In another embodiment, with respect to compounds of formulae Ia-Ic, R¹ is Me.

In one embodiment, with respect to compounds of formulae Ia-Ic, R² is C₁-C₆ alkyl or cycloalkyl.

In another embodiment, with respect to compounds of formulae Ia-Ic, R² is Me, Et, n-Pr, t-Bu, cyclopropyl, cyclohexyl, or cyclopentyl.

In one embodiment, with respect to compounds of formulae Ia-Ib, R⁴ is C₁-C₆ alkyl.

In another embodiment, with respect to compounds of formulae Ia-1b, R⁴ is Me, Et, n-Pr, i-Pr, n-Bu, t-Bu, n-pentyl, CH₂CH(Me)Me, CH₂CH₂-t-Bu, CH₂CH(Me)CH₂Me, CH₂CH(Et)CH₂Me, or CH₂CH(Me)CH₂CH₂Me.

In one embodiment, with respect to compounds of formula Ib, m1 is 0, 1 or 2. In another embodiment, m1 is 1 or 2. In yet another embodiment m1 is 1.

In one embodiment, with respect to compounds of formula Ia, the compound is according to formula II:

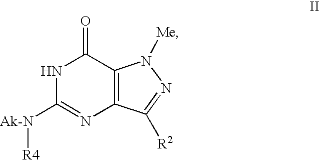

wherein R² is C₁-C₆ alkyl or cycloalkyl and R⁴ is C₁-C₆ alkyl.

In one embodiment, with respect to compounds of formula II, R² is C₁-C₆ alkyl. In another embodiment R² is Me, Et, n-Pr, t-Bu. In yet another embodiment R² is n-Pr.

In one embodiment, with respect to compounds of formula II, R² is cycloalkyl. In another embodiment, R² is cyclopropyl, cyclohexyl, or cyclopentyl.

In one embodiment, with respect to compounds of formula II, R⁴ is alkyl. In another embodiment R⁴ is Me, Et, n-Pr, i-Pr, n-Bu, t-Bu, n-pentyl, CH₂CH(Me)Me, CH₂CH₂-t-Bu, CH₂CH(Me)CH₂Me, CH₂CH(Et)CH₂Me, or CH₂CH(Me)CH₂CH₂Me. In yet another embodiment R⁴ is n-Pr.

In one embodiment, with respect to compounds of formula II, Ak is Me, Et, n-Pr, i-Pr, n-Bu, t-Bu, n-pentyl, CH₂CH(Me)Me, CH₂CH₂-t-Bu, CH₂CH(Me)CH₂Me, CH₂CH(Et)CH₂Me, or CH₂CH(Me)CH₂CH₂Me. In yet another embodiment Ak is n-Pr.

In one embodiment, with respect to compounds of formula II, each of R², R⁴, and Ak is n-Pr.

In one embodiment, with respect to compounds of formula I, the compound is according to formula Ib.

In one embodiment, with respect to compounds of formula Ib, Cy1 is substituted or unsubstituted aryl.

In one embodiment, with respect to compounds of formula Ib, m1 is 1 or 2.

In one embodiment, with respect to compounds of formula Ib, the compound is according to formulae IIIa or IIIb:

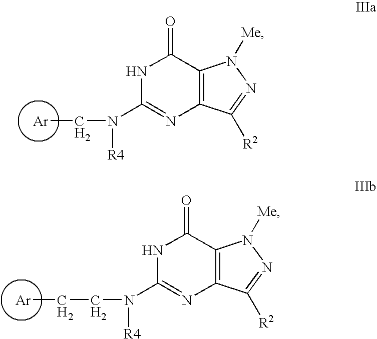

wherein:

Ar is substituted or unsubstituted phenyl;

R² is C₁-C₆ alkyl or cycloalkyl; and

R⁴ is alkyl.

In one embodiment, with respect to compounds of formulae IIa or IIIb, Ar is unsubstituted phenyl.

In another embodiment, with respect to compounds of formulae IIa or IIIb, Ar is phenyl substituted with one or more groups selected from C₁-C₆ alkyl, halo, C₁-C₆ haloalkyl, and C₁-C₆ alkoxy.

In another embodiment Ar is phenyl substituted with one or more groups selected from Me, Et, Cl, F, CF₃, OMe, and OEt.

In another embodiment, with respect to compounds of formulae IIa or IIIb, Ar is phenyl substituted with cycloalkyl, heterocycloalkyl, aryl or heteroaryl.

In another embodiment, with respect to compounds of formulae IIIa or IIIb, Ar is phenyl substituted with piperidinyl, piperazinyl, and morpholinyl.

In another embodiment, with respect to compounds of formulae IIIa or IIIb, Ar is phenyl substituted with pyrrolyl, pyrazolyl, imidazolyl, triazolyl, tetrazolyl, thiazolyl, and oxazolyl.

In another embodiment, with respect to compounds of formulae IIIa or IIIb, Ar is phenyl substituted with pyridyl and pyrimidinyl.

In another embodiment, with respect to compounds of formula IIIa, the compound is according to formula IVa:

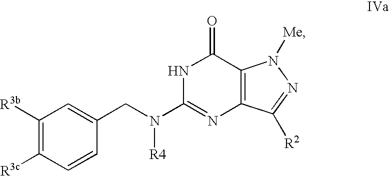

wherein:

R² and R⁴ are as described for formula IIIa; and

each of R^{3b} and R^{3c} is independently H, C₁-C₆ alkyl, halo, C₁-C₆ haloalkyl, or C₁-C₆ alkoxy.

In one embodiment, with respect to compounds of formula IVa, one of R^{3b} and R^{3c} is Me, F, Cl, CF₃, OMe, or OEt and the other is H.

In one embodiment, with respect to compounds of formula IVa, R^{3b} is H; and R^{3c} is Me, F, Cl, CF₃, OMe, or OEt.

In one embodiment, with respect to compounds of formula IVa, R^{3b} is Me, F, Cl, CF₃, OMe, or OEt; and R^{3c} is H.

In one embodiment, with respect to compounds of formula IVa, both R^{3b} and R^{3c} are independently F, Cl, OMe, CF₃, or OEt. In another embodiment, both R^{3b} and R^{3c} are Cl. In another embodiment, both R^{3b} and R^{3c} are F. In another embodiment, both R^{3b} and R^{3c} are OMe. In another embodiment, both R^{3b} and R^{3c} are CF₃.

In one embodiment, with respect to compounds of formula IVa, both R^{3c} is H.

In another embodiment, with respect to compounds of formula IIIa, the compound is according to formula IVb:

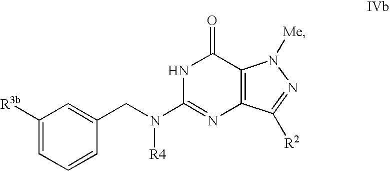

wherein:

R² and R⁴ are as described for formula IIIa; and

R^{3b} is substituted or unsubstituted aryl, heterocycloalkyl or heteroaryl.

In one embodiment, with respect to compounds of formula IVb, R^{3b} is selected from substituted or unsubstituted piperidinyl, piperazinyl, and morpholinyl.

In one embodiment, with respect to compounds of formula IVb, R^{3b} is selected from substituted or unsubstituted pyrrolyl, pyrazolyl, imidazolyl, triazolyl, tetrazolyl, thiazolyl, and oxazolyl.

In one embodiment, with respect to compounds of formula IVb, R^{3b} is selected from substituted or unsubstituted pyridyl, and pyrimidinyl.

In one embodiment, with respect to compounds of formula IVb, the compound is according to formulae Va, Vb, Vc, Vd, Ve, Vf, Vg, and Vh:

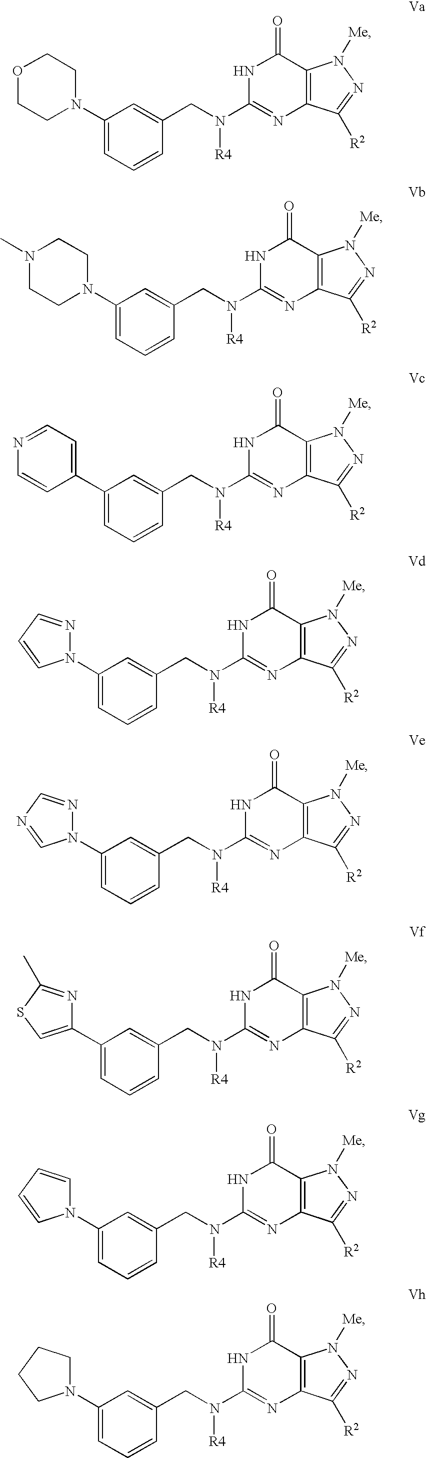

wherein R² and R⁴ are as described for formula IIIa.

In one embodiment, with respect to compounds of formula Ib, Cy1 is substituted or unsubstituted heteroaryl.

In one embodiment, with respect to compounds of formula Ib, the compound is according to formulae VIa or VIb:

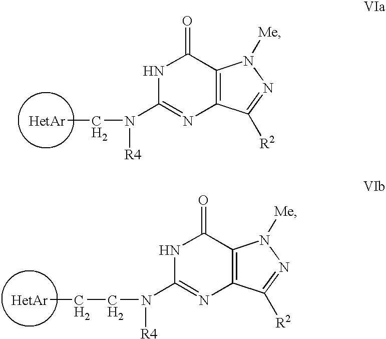

wherein:

HetAr is substituted or unsubstituted heteroaryl;

R² is C₁-C₆ alkyl or cycloalkyl;

R⁴ is C₁-C₆ alkyl.

In one embodiment, with respect to compounds of formulae VIa or VIb, HetAr is unsubstituted pyridyl. In another embodiment, HetAr is pyridyl substituted with one or more groups selected from C₁-C₆ alkyl, halo, C₁-C₆ haloalkyl, and C₁-C₆ alkoxy.

In one embodiment, with respect to compounds of formulae VIa or VIb, HetAr is pyridyl substituted with cycloalkyl, heterocycloalkyl, aryl or heteroaryl.

In one embodiment, with respect to compounds of formula VIa, the compound is according to formula VIIa or VIIIb:

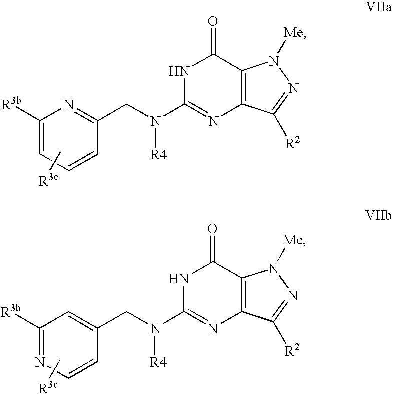

wherein:

R² and R⁴ are as described for formula VIa; and

each of R^{3b} and R^{3c} is independently H, C₁-C₆ alkyl, halo, C₁-C₆ haloalkyl, or C₁-C₆ alkoxy.

In one embodiment, with respect to compounds of formulae VIIa or VIIb, one of R^{3b} and R^{3c} is Me, F, Cl, CF₃, OMe, or OEt and the other is H.

In one embodiment, with respect to compounds of formulae VIIa or VIIb, both R^{3b} and R^{3c} are independently F, Cl, OMe, CF₃, or OEt.

In one embodiment, with respect to compounds of formulae VIIa or VIIb, the compound is according to formulae VIIIa or VIIIb:

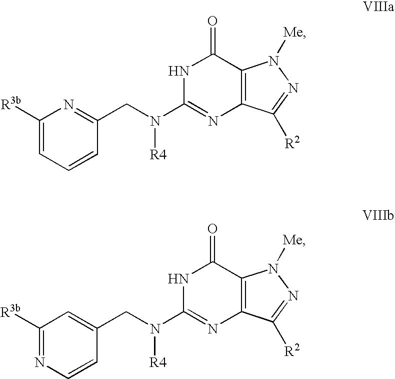

wherein:

R² and R⁴ are as described for formula VIa; and

R^{3b} is substituted or unsubstituted aryl, heterocycloalkyl or heteroaryl.

In one embodiment, with respect to compounds of formulae VIIa or VIIIb, R^{3b} is selected from substituted or unsubstituted piperidinyl, piperazinyl, and morpholinyl.

In one embodiment, with respect to compounds of formulae VIIa or VIIIb, R^{3b} is selected from substituted or unsubstituted pyrrolyl, pyrazolyl, imidazolyl, triazolyl, tetrazolyl, thiazolyl, and oxazolyl.

In one embodiment, with respect to compounds of formulae VIIa or VIIIb, R^{3b} is selected from substituted or unsubstituted pyridyl, and pyrimidinyl.

In one embodiment, with respect to compounds of formula VIIa, the compound is according to formula IXa, IXb, IXc, IXd, IXe, Ixf, IXg, and IXh:

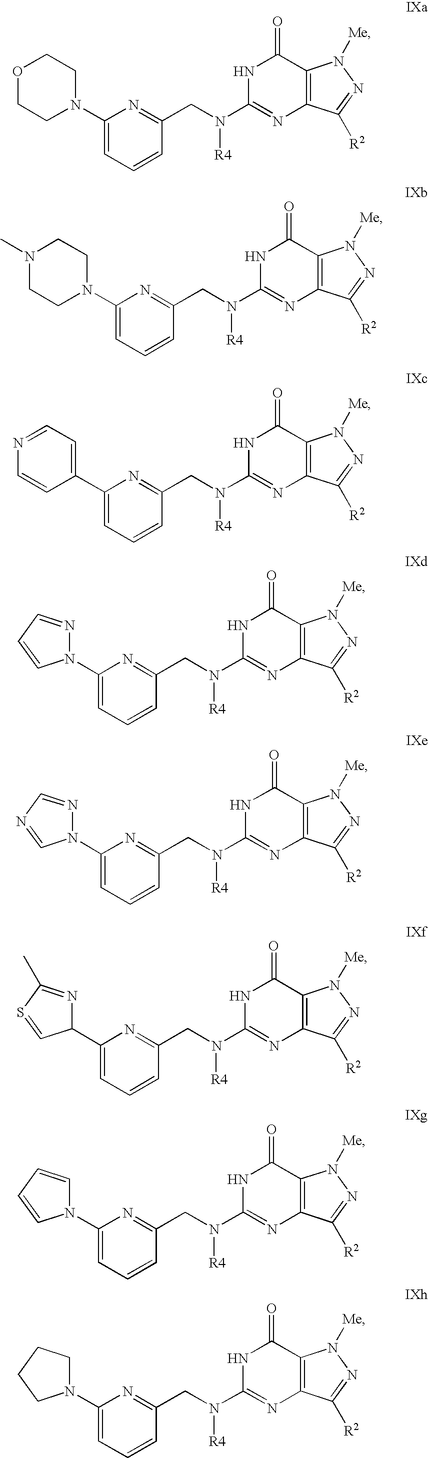

wherein R² and R⁴ are as described for formula VIa.

In one embodiment, with respect to compounds of formula VIIIb, the compound is according to formula Xa, Xb, Xc, Xd, Xe, Xf, Xg, and Xh:

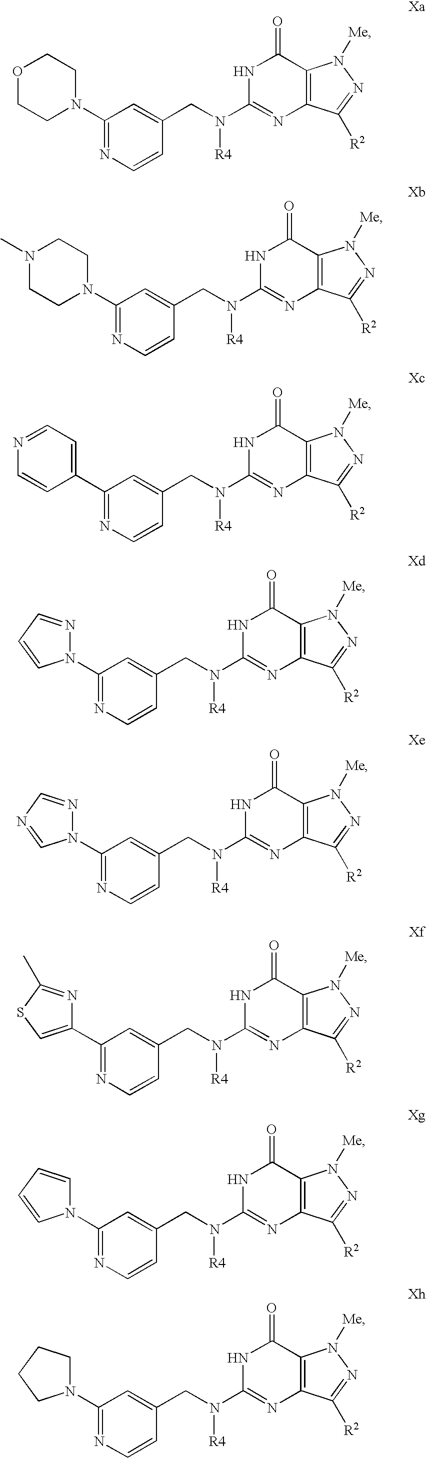

wherein R² and R⁴ are as described for formula VIa.

In one embodiment, with respect to compounds of formulae VIa or VIb, HetAr is selected from substituted or unsubstituted pyrrolyl, pyrazolyl, imidazolyl, triazolyl, tetrazolyl, thiazolyl, isoxazolyl, and oxazolyl.

In one embodiment, with respect to compounds of formulae VIa or VIb, HetAr is selected from pyrrolyl, pyrazolyl, imidazolyl, triazolyl, tetrazolyl, thiazolyl, isoxazolyl, and oxazolyl; substituted with one or more groups selected from C₁-C₆ alkyl, halo, haloalkyl, and phenyl. In one embodiment, the HetAr is mono substituted and the substitution is selected from Ph, Me, CF₃, and halo. In another embodiment, the HetAr is di substituted and the substitution is selected from Ph, Me and CF₃.

In one embodiment, with respect to compounds of formula VIa, the compound is according to formulae XIa, XIb, XIc, XId, and XIe:

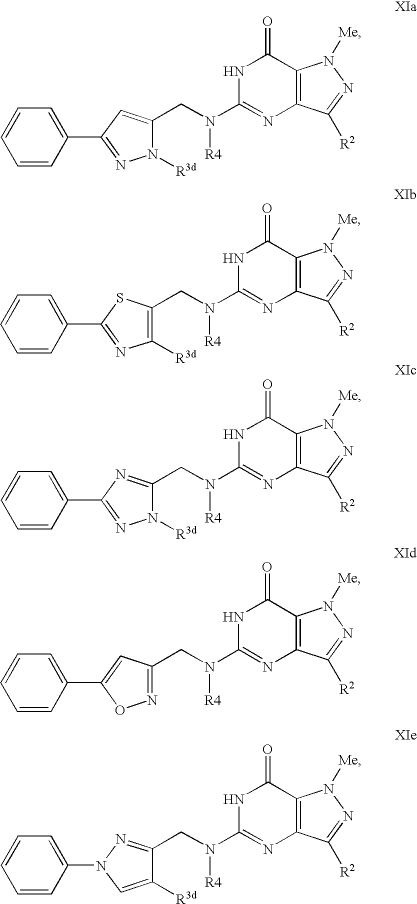

wherein R² and R⁴ are as as described for formula VIa; and R^{3d} is H, C₁-C₆ alkyl or C₁-C₆ haloalkyl.

In one embodiment, with respect to compounds of formulae XIa-XIc and XIe wherein R^{3d} is Me.

In one embodiment, with respect to compounds of formulae IIIa-XIe, R² is Me, Et, n-Pr, t-Bu, cyclopropyl, cyclohexyl, or cyclopentyl.

In one embodiment, with respect to compounds of formulae IIIa-XIe, R⁴ is Me, Et, n-Pr, i-Pr, n-Bu, t-Bu, n-pentyl, CH₂CH(Me)Me, CH₂CH₂-t-Bu, CH₂CH(Me)CH₂Me, CH₂CH(Et)CH₂Me, or CH₂CH(Me)CH₂CH₂Me.

In one embodiment, with respect to compounds of formulae IIIa-XIe, each of R² and R⁴ is n-Pr.

In one embodiment, with respect to compounds of formulae IIIa-XIe, each of R², R³, and R⁴ is n-Pr.

In one embodiment, with respect to compounds of formula I, the compound is according to formula Ic.

In one embodiment, with respect to compounds of formula Ic, the compound is according to formula XII:

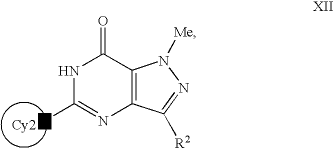

wherein Cy2, and R² are as described for formula Ic.

In one embodiment, with respect to compounds of formula XII, Cy2 is selected from substituted or unsubstituted piperidine, piperazine, s diazepine, morpholine, or tetrahydroisoquinoline.

In one embodiment, with respect to compounds of formula XII, R² is Me, Et, n-Pr, t-Bu, cyclopropyl, cyclohexyl, or cyclopentyl.

In one embodiment, with respect to compounds of formula I, the compound is selected from
5-(4-(2-chloro-6-fluorobenzyl)-1,4-diazepan-1-yl)-1-methyl-3-propyl-1H-pyrazolo[4,3-d]pyrimidin-7(6H)-one;
5-((3,4-dichlorobenzyl)(methyl)amino)-1-methyl-3-propyl-1H-pyrazolo[4,3-d]pyrimidin-7(6H)-one;
1-methyl-5-(methyl(4-(piperidin-1-yl)benzyl)amino)-3-propyl-1H-pyrazolo[4,3-d]pyrimidin-7(6H)-one;
5-(dipropylamino)-1-methyl-3-propyl-1H-pyrazolo[4,3-d]pyrimidin-7(6H)-one;
1-methyl-5-(methyl(3-(trifluoromethyl)benzyl)amino)-3-propyl-H-pyrazolo[4,3-d]pyrimidin-7(6H)-one;
1-methyl-5-(4-(4-methylpiperazin-1-yl)benzylamino)-3-propyl-1H-pyrazolo[4,3-d]pyrimidin-7(6H)-one;
5-(1H-indazol-5-ylamino)-1-methyl-3-propyl-1H-pyrazolo[4,3-d]pyrimidin-7(6H)-one;
5-(3,4-dihydroisoquinolin-2(1H)-yl)-1-methyl-3-propyl-1H-pyrazolo[4,3-d]pyrimidin-7(6H)-one;
5-((4-methoxybenzyl)(methyl)amino)-1-methyl-3-propyl-1H-pyrazolo[4,3-d]pyrimidin-7(6H)-one;
1-methyl-5-(methyl(3-morpholinobenzyl)amino)-3-propyl-1H-pyrazolo[4,3-d]pyrimidin-7(6H)-one;
1-methyl-5-(3-(piperidin-1-yl)benzylamino)-3-propyl-1H-pyrazolo[4,3-d]pyrimidin-7(6H)-one;
1-methyl-5-(methyl(3-(pyrimidin-5-yl)benzyl)amino)-3-propyl-1H-pyrazolo[4,3-d]pyrimidin-7(6H)-one;
1-methyl-5-(methyl(4-(pyrimidin-5-yl)benzyl)amino)-3-propyl-1H-pyrazolo[4,3-d]pyrimidin-7(6H)-one;
5-(((1,5-dimethyl-1H-pyrazol-3-yl)methyl)(methyl)amino)-1-methyl-3-propyl-1H-pyrazolo[4,3-d]pyrimidin-7(6H)-one;
5-((3-chlorobenzyl)(methyl)amino)-1-methyl-3-propyl-1H-pyrazolo[4,3-d]pyrimidin-7(6H)-one;
1-methyl-5-(methyl(4-(trifluoromethyl)benzyl)amino)-3-propyl-1H-pyrazolo[4,3-d]pyrimidin-7(6H)-one;
5-((4-chlorobenzyl)(methyl)amino)-1-methyl-3-propyl-1H-pyrazolo[4,3-d]pyrimidin-7(6H)-one;
5-(3-fluorobenzylamino)-1-methyl-3-propyl-1H-pyrazolo[4,3-d]pyrimidin-7(6H)-one;
5-(isopentylamino)-1-methyl-3-propyl-1H-pyrazolo[4,3-d]pyrimidin-7(6H)-one;
5-((3,4-dimethoxyphenethyl)(methyl)amino)-1-methyl-3-propyl-1H-pyrazolo[4,3-d]pyrimidin-7(6H)-one;
5-((4-fluorobenzyl)(methyl)amino)-1-methyl-3-propyl-1H-pyrazolo[4,3-d]pyrimidin-7(6H)-one;
1-methyl-S-(methyl(phenethyl)amino)-3-propyl-1H-pyrazolo[4,3-d]pyrimidin-7(6H)-one;
5-(4-fluorobenzylamino)-1-methyl-3-propyl-1H-pyrazolo[4,3-d]pyrimidin-7(6H)-one;
5-((2-chlorobenzyl)(methyl)amino)-1-methyl-3-propyl-1H-pyrazolo[4,3-d]pyrimidin-7(6H)-one;
1-methyl-5-(methyl((5-phenylisoxazol-3-yl)methyl)amino)-3-propyl-1H-pyrazolo[4,3-d]pyrimidin-7(6H)-one;
1-methyl-3-propyl-5-(3-(pyrrolidin-1-yl)benzylamino)-1H-pyrazolo[4,3-d]pyridin-7(6H)-one;
1-methyl-5-(4-(morpholinomethyl)benzylamino)-3-propyl-1H-pyrazolo[4,3-d]pyrimidin-7(6H)-one;
5-(benzyl(isopropyl)amino)-1-methyl-3-propyl-1H-pyrazolo[4,3-d]pyrimidin-7(6H)-one;
5-(diisobutylamino)-1-methyl-3-propyl-1H-pyrazolo[4,3-d]pyrimidin-7(6H)-one;
1-methyl-5-(methyl(propyl)amino)-3-propyl-1H-pyrazolo[4,3-d]pyrimidin-7(6H)-one;
5-(3,5-bis(trifluoromethyl)benzylamino)-1-methyl-3-propyl-1H-pyrazolo[4,3-d]pyrimidin-7(6H)-one;
5-(3,4-dichlorobenzylamino)-1-methyl-3-propyl-1H-pyrazolo[4,3-d]pyrimidin-7(6H)-one;
5-(benzyl(methyl)amino)-1-methyl-3-propyl-1H-pyrazolo[4,3-d]pyrimidin-7(6H)-one;
5-(6-fluoro-3,4-dihydroisoquinolin-2(1H)-yl)-1-methyl-3-propyl-1H-pyrazolo[4,3-d]pyrimidin-7(6H)-one;
1-methyl-5-(methyl((1-methyl-1H-indazol-3-yl)methyl)amino)-3-propyl-1H-pyrazolo[4,3-d]pyrimidin-7(6H)-one;
1-methyl-5-(methyl(thieno[2,3-b]pyridin-2-ylmethyl)amino)-3-propyl-1H-pyrazolo[4,3-d]pyrimidin-7(6H)-one;
5-(6,7-dimethoxy-1-methyl-3,4-dihydroisoquinolin-2(1H)-yl)-1-methyl-3-propyl-1H-pyrazolo[4,3-d]pyrimidin-7(6H)-one;
1-methyl-5-(methyl((1-methyl-3-phenyl-1H-pyrazol-5-yl)methyl)amino)-3-propyl-1H-pyrazolo[4,3-d]pyrimidin-7(6H)-one;
1-methyl-5-(methyl((2-(4-(trifluoromethyl)phenyl)thiazol-4-yl)methyl)amino)-3-propyl-1H-pyrazolo[4,3-d]pyrimidin-7(6H)-one;
1-methyl-5-(methyl(4-(pyridin-4-yl)benzyl)amino)-3-propyl-1H-pyrazolo[4,3-d]pyrimidin-7(6H)-one;
1-methyl-5-(methyl((2-(pyrrolidin-1-yl)pyridin-4-yl)methyl)amino)-3-propyl-1H-pyrazolo[4,3-d]pyrimidin-7(6H)-one;
1-methyl-5-(methyl((6-morpholinopyridin-2-yl)methyl)amino)-3-propyl-1H-pyrazolo[4,3-d]pyrimidin-7(6H)-one;
1-methyl-5-(methyl(3-(pyridin-4-yl)benzyl)amino)-3-propyl-1H-pyrazolo[4,3-d]pyrimidin-7(6H)-one;
1-methyl-5-(methyl(4-(pyridin-2-yl)benzyl)amino)-3-propyl-1H-pyrazolo[4,3-d]pyrimidin-7(6H)-one;
5-((3,4-dichlorobenzyl)(isopentyl)amino)-1-methyl-3-propyl-1H-pyrazolo[4,3-d]pyrimidin-7(6H)-one;
5-(butyl(3,4-dichlorobenzyl)amino)-1-methyl-3-propyl-1H-pyrazolo[4,3-d]pyrimidin-7(6H)-one;
1-methyl-5-(methyl((4-methyl-2-phenylthiazol-5-yl)methyl)amino)-3-propyl-1H-pyrazolo[4,3-d]pyrimidin-7(6H)-one;
1-methyl-5-(methyl(3-(2-morpholinoethoxy)benzyl)amino)-3-propyl-1H-pyrazolo[4,3-d]pyrimidin-7(6H)-one;
1-methyl-5-(methyl(4-(2-morpholinoethoxy)benzyl)amino)-3-propyl-1H-pyrazolo[4,3-d]pyrimidin-7(6H)-one;
1-methyl-5-(2-(2-morpholinoethoxy)benzylamino)-3-propyl-1H-pyrazolo[4,3-d]pyrimidin-7(6H)-one;
1-methyl-5-((5-methyl-3-phenylisoxazol-4-yl)methylamino)-3-propyl-1H-pyrazolo[4,3-d]pyrimidin-7(6H)-one;
1-methyl-5-((5-methyl-2-phenyl-2H-1,2,3-triazol-4-yl)methylamino)-3-propyl-1H-pyrazolo[4,3-d]pyrimidin-7(6H)-one;
1-methyl-5-(3-(morpholinomethyl)benzylamino)-3-propyl-1H-pyrazolo[4,3-d]pyrimidin-7(6H)-one;
1-methyl-5-((2-morpholinopyridin-4-yl)methylamino)-3-propyl-1H-pyrazolo[4,3-d]pyrimidin-7(6H)-one;
1-methyl-5-(3-(2-morpholinoethoxy)benzylamino)-3-propyl-1H-pyrazolo[4,3-d]pyrimidin-7(6H)-one;
1-methyl-5-(3-morpholinobenzylamino)-3-propyl-1H-pyrazolo[4,3-d]pyrimidin-7(6H)-one;
1-methyl-5-((1-phenyl-1H-pyrazol-3-yl)methylamino)-3-propyl-1H-pyrazolo[4,3-d]pyrimidin-7(6H)-one;
1-methyl-5-(methyl(naphthalen-1-ylmethyl)amino)-3-propyl-1H-pyrazolo[4,3-d]pyrimidin-7(6H)-one;
5-(4-(2,2-diphenylacetoyl)piperazin-1-yl)-1-methyl-3-propyl-1H-pyrazolo[4,3-d]pyrimidin-7(6H)-one;
1-methyl-3-propyl-5-(4-(trifluoromethyl)piperidin-1-yl)-1H-pyrazolo[4,3-d]pyrimidin-7(6H)-one;
1-methyl-5-(methyl(4-morpholinobenzyl)amino)-3-propyl-1H-pyrazolo[4,3-d]pyrimidin-7(6H)-one;
5-((2,3-dihydrobenzofuran-5-yl)methylamino)-1-methyl-3-propyl-1H-pyrazolo[4,3-d]pyrimidin-7(6H)-one;
5-(1H-pyrazol-1-yl)benzylamino)-1-methyl-3-propyl-1H-pyrazolo[4,3-d]pyrimidin-7(6H)-one;
5-(1H-pyrrol-1-yl)benzylamino)-1-methyl-3-propyl-1H-pyrazolo[4,3-d]pyrimidin-7(6H)-one;
1-methyl-5-(methyl(3-(2-methylthiazol-4-yl)benzyl)amino)-3-propyl-1H-pyrazolo[4,3-d]pyrimidin-7(6H)-one;
1-methyl-5-((1-methyl-3-phenyl-1H-pyrazol-5-yl)methylamino)-3-propyl-1H-pyrazolo[4,3-d]pyrimidin-7(6H)-one;
1-methyl-5-(methyl-1H-pyrazol-3-yl)benzylamino)-3-propyl-1H-pyrazolo[4,3-d]pyrimidin-7(6H)-one;
1-methyl-5-((1-methyl-1H-indol-5-yl)methylamino)-3-propyl-1H-pyrazolo[4,3-d]pyrimidin-7(6H)-one;
1-methyl-5-((1-methyl-1H-indol-6-yl)methylamino)-3-propyl-1H-pyrazolo[4,3-d]pyrimidin-7(6H)-one;
3-cyclopentyl-5-((4-fluorobenzyl)(methyl)amino)-1-methyl-1H-pyrazolo[4,3-d]pyrimidin-7(6H)-one;
5-((4-chlorobenzyl)(methyl)amino)-3-cyclopentyl-1-methyl-1H-pyrazolo[4,3-d]pyrimidin-7(6H)-one;
1-methyl-5-((4-methyl-2-phenylthiazol-5-yl)methylamino)-3-propyl-1H-pyrazolo[4,3-d]pyrimidin-7(6H)-one;
5-(1H-1,2,4-triazol-1-yl)benzylamino)-1-methyl-3-propyl-1H-pyrazolo[4,3-d]pyrimidin-7(6H)-one;
5-(isobutyl(3-morpholinobenzyl)amino)-1-methyl-3-propyl-1H-pyrazolo[4,3-d]pyrimidin-7(6H)-one;
5-((4-chlorophenethyl)(propyl)amino)-1-methyl-3-propyl-1H-pyrazolo[4,3-d]pyrimidin-7(6H)-one;
3-cyclopentyl-5-(3,4-dichlorobenzylamino)-1-methyl-1H-pyrazolo[4,3-d]pyrimidin-7(6H)-one;
3-cyclopentyl-5-((3,4-dichlorobenzyl)(methyl)amino)-1-methyl-1H-pyrazolo[4,3-d]pyrimidin-7(6H)-one;
3-cyclopentyl-5-(dipropylamino)-1-methyl-1H-pyrazolo[4,3-d]pyrimidin-7(6H)-one;
5-(4-chlorophenethylamino)-1-methyl-3-propyl-1H-pyrazolo[4,3-d]pyrimidin-7(6H)-one;
3-tert-butyl-1-methyl-5-(methyl((4-methyl-2-phenylthiazol-5-yl)methyl)amino)-1H-pyrazolo[4,3-d]pyrimidin-7(6H)-one;
3-tert-butyl-1-methyl-5-(methyl((1-methyl-3-phenyl-1H-pyrazol-5-yl)methyl)amino)-1H-pyrazolo[4,3-d]pyrimidin-7(6H)-one;
5-(3,4-diethoxyphenethylamino)-1-methyl-3-propyl-1H-pyrazolo[4,3-d]pyrimidin-7(6H)-one;
5-([1,2,4]triazolo[4,3-a]pyridin-3-ylmethylamino)-1-methyl-3-propyl-1H-pyrazolo[4,3-d]pyrimidin-7(6H)-one;
3-cyclopentyl-1-methyl-5-(2-methylpiperidin-1-yl)-1H-pyrazolo[4,3-d]pyrimidin-7(6H)-one;
3-cyclopentyl-1-methyl-5-(piperidin-1-yl)-1H-pyrazolo[4,3-d]pyrimidin-7(6H)-one;
3-cyclopentyl-1-methyl-5-(2-phenylpyrrolidin-1-yl)-1H-pyrazolo[4,3-d]pyrimidin-7(6H)-one;
5-(4-fluorophenethylamino)-1-methyl-3-propyl 1H-pyrazolo[4,3-d]pyrimidin-7(6H)-one;
1-methyl-5-(2-(1-phenyl-1H-pyrazol-4-yl)ethylamino)-3-propyl-1H-pyrazolo[4,3-d]pyrimidin-7(6H)-one;
3-tert-butyl-5-(dipropylamino)-1-methyl-1H-pyrazolo[4,3-d]pyrimidin-7(6H)-one;
3-tert-butyl-1-methyl-5-(methyl(3-(2-methylthiazol-4-yl)benzyl)amino)-1H-pyrazolo[4,3-d]pyrimidin-7(6H)-one;
5-((1-benzyl-3,5-dimethyl-1H-pyrazol-4-yl)methylamino)-1-methyl-3-propyl-1H-pyrazolo[4,3-d]pyrimidin-7(6H)-one;
3-tert-butyl-1-methyl-5-(3-(4-methylpiperazin-1-yl)benzylamino)-1H-pyrazolo[4,3-d]pyrimidin-7(6H)-one;
5-((1H-pyrazol-1-yl)benzyl)(isobutyl)amino)-3-tert-butyl-1-methyl-1H-pyrazolo[4,3-d]pyrimidin-7(6H)-one;
5-((1H-pyrazol-1-yl)benzyl)(isobutyl)amino)-1-methyl-3-propyl-1H-pyrazolo[4,3-d]pyrimidin-7(6H)-one;
5-((1H-1,2,4-triazol-1-yl)benzyl)(isobutyl)amino)-3-tert-butyl-1-methyl-1H-pyrazolo[4,3-d]pyrimidin-7(6H)-one;
5-((1H-1,2,4-triazol-1-yl)benzyl)(isobutyl)amino)-1-methyl-3-propyl-1H-pyrazolo[4,3-d]pyrimidin-7(6H)-one;
3-tert-butyl-5-((4-fluorobenzyl)(isobutyl)amino)-1-methyl-1H-pyrazolo[4,3-d]pyrimidin-7(6H)-one;
5-((4-fluorobenzyl)(isobutyl)amino)-1-methyl-3-propyl-1H-pyrazolo[4,3-d]pyrimidin-7(6H)-one;
3-tert-butyl-5-(isobutyl(methyl)amino)-1-methyl-1H-pyrazolo[4,3-d]pyrimidin-7(6H)-one;
5-((1H-pyrrol-1-yl)benzyl)(isobutyl)amino)-3-tert-butyl-1-methyl-1H-pyrazolo[4,3-d]pyrimidin-7(6H)-one;
5-((1H-pyrrol-1-yl)benzyl)(isobutyl)amino)-1-methyl-3-propyl-1H-pyrazolo[4,3-d]pyrimidin-7(6H)-one;
5-(isobutyl(3-(4-methylpiperazin-1-yl)benzyl)amino)-1-methyl-3-propyl-1H-pyrazolo[4,3-d]pyrimidin-7(6H)-one;
3-tert-butyl-5-((3,3-dimethylbutyl)(3-morpholinobenzyl)amino)-1-methyl-1H-pyrazolo[4,3-d]pyrimidin-7(6H)-one;
3-tert-butyl-1-methyl-5-((2-methylpentyl)(3-morpholinobenzyl)amino)-1H-pyrazolo[4,3-d]pyrimidin-7(6H)-one;
3-tert-butyl-5-((2-ethylbutyl)(methyl)amino)-1-methyl-1H-pyrazolo[4,3-d]pyrimidin-7(6H)-one;
5-((1H-pyrazol-1-yl)benzyl)(isopentyl)amino)-1-methyl-3-propyl-1H-pyrazolo[4,3-d]pyrimidin-7(6H)-one;
5-((1H-pyrazol-1-yl)benzyl)(2-ethylbutyl)amino)-1-methyl-3-propyl-1H-pyrazolo[4,3-d]pyrimidin-7(6H)-one;
5-((3,3-dimethylbutyl)(3-morpholinobenzyl)amino)-1-methyl-3-propyl-1H-pyrazolo[4,3-d]pyrimidin-7(6H)-one;
3-tert-butyl-5-((2-ethylbutyl)(3-morpholinobenzyl)amino)-1-methyl-1H-pyrazolo[4,3-d]pyrimidin-7(6H)-one;
5-((3,3-dimethylbutyl)(methyl)amino)-1-methyl-3-propyl-1H-pyrazolo[4,3-d]pyrimidin-7(6H)-one;
3-tert-butyl-5-((3,3-dimethylbutyl)(methyl)amino)-1-methyl-1H-pyrazolo[4,3-d]pyrimidin-7(6H)-one;
1-methyl-5-(methyl(2-methylbutyl)amino)-3-propyl-1H-pyrazolo[4,3-d]pyrimidin-7(6H)-one;
1-methyl-5-((2-methylpentyl)(3-morpholinobenzyl)amino)-3-propyl-1H-pyrazolo[4,3-d]pyrimidin-7(6H)-one;
1-methyl-5-((2-methylbutyl)(3-morpholinobenzyl)amino)-3-propyl-1H-pyrazolo[4,3-d]pyrimidin-7(6H)-one;
3-tert-butyl-1-methyl-5-(methyl(2-methylpentyl)amino)-1H-pyrazolo[4,3-d]pyrimidin-7(6H)-one;
5-((1H-1,2,4-triazol-1-yl)benzyl)(2-methylbutyl)amino)-1-methyl-3-propyl-1H-pyrazolo[4,3-d]pyrimidin-7(6H)-one;
3-tert-butyl-1-methyl-5-((2-methylbutyl)(3-morpholinobenzyl)amino)-1H-pyrazolo[4,3-d]pyrimidin-7(6H)-one;
5-((1H-1,2,4-triazol-1-yl)benzyl)(2-methylbutyl)amino)-3-tert-butyl-1-methyl-1H-pyrazolo[4,3-d]pyrimidin-7(6H)-one;
5-((1H-pyrazol-1-yl)benzyl)(neopentyl)amino)-1-methyl-3-propyl-1H-pyrazolo[4,3-d]pyrimidin-7(6H)-one;
4-((1-methyl-7-oxo-3-propyl-6,7-dihydro-1H-pyrazolo[4,3-d]pyrimidin-5-ylamino)methyl)benzenesulfonamide;
5-((1H-pyrazol-1-yl)benzyl)(2-ethylbutyl)amino)-3-tert-butyl-1-methyl-1H-pyrazolo[4,3-d]pyrimidin-7(6H)-one;
5-((1H-pyrazol-1-yl)benzyl)(2-methylpentyl)amino)-3-tert-butyl-1-methyl-1H-pyrazolo[4,3-d]pyrimidin-7(6H)-one;
1-methyl-5-(methyl(2-methylpentyl)amino)-3-propyl-1H-pyrazolo[4,3-d]pyrimidin-7(6H)-one;
3-tert-butyl-1-ethyl-5-(isobutyl(3-morpholinobenzyl)amino)-1H-pyrazolo[4,3-d]pyrimidin-7(6H)-one; and
3-cyclopropyl-5-(dipropylamino)-1-methyl-1H-pyrazolo[4,3-d]pyrimidin-7(6H)-one;
   or a pharmaceutically acceptable salt, hydrate, solvate or prodrug thereof or isotopic variants thereof, stereoisomers or tautomers thereof.

In yet another embodiment, with respect to compounds of formulae Ia-Ic, the compound is selected from all compounds of the invention exemplified specifically herein.

A compound for use according to the invention may contain one or more asymmetric carbon atoms and may exist in racemic and optically active forms. It will be understood by a person of skill in the art that the present invention includes both the racemic mixture and each enantiomer in isolated form. A compound according to an embodiment of the invention may be in trans or cis form.

The present invention also extends to a prodrug of a compound according to an embodiment of the invention such as an ester or amide thereof. A prodrug is a compound that may be converted under physiological conditions or by solvolysis to a compound according to an embodiment of the invention or to a pharmaceutically acceptable salt of a compound according to an embodiment of the invention. A prodrug may be inactive when administered to a subject but is converted in vivo to an active compound of the invention. ‘Pharmaceutically acceptable prodrugs’ as used herein refers to those prodrugs of the compounds useful in the present invention, which are, within the scope of sound medical judgment, suitable for use in contact with the tissues of patients with undue toxicity, irritation, allergic response commensurate with a reasonable benefit/risk ratio, and effective for their intended use of the compounds of the invention. The term ‘prodrug’ means a compound that is transformed in vivo to yield an effective compound useful in the present invention or a pharmaceutically acceptable salt, hydrate or solvate thereof. The transformation may occur by various mechanisms, such as through hydrolysis in blood. The compounds bearing metabolically cleavable groups have the advantage that they may exhibit improved bioavailability as a result of enhanced solubility and/or rate of absorption conferred upon the parent compound by virtue of the presence of the metabolically cleavable group, thus, such compounds act as prodrugs. A thorough discussion is provided in Design of Prodrugs, H. Bundgard, ed., Elsevier (1985); Methods in Enzymology; K. Widder et al, Ed., Academic Press, 42, 309-396 (1985); A Textbook of Drug Design and Development, Krogsgaard-Larsen and H. Bundgard, ed., Chapter 5; “Design and Applications of Prodrugs”113-191 (1991); Advanced Drug Delivery Reviews, H. Bundgard, 8, 1-38, (1992); J. Pharm. Sci., 77,285 (1988); Chem. Pharm. Bull., N. Nakeya et al, 32, 692 (1984); Pro-drugs as Novel Delivery Systems, T. Higuchi and V. Stella, 14 A.C.S. Symposium Series, and Bioreversible Carriers in Drug Design, E. B. Roche, ed., American Pharmaceutical Association and Pergamon Press, 1987, all of which are incorporated herein by reference.

### Pharmaceutical Compositions

Compounds of the invention can be incorporated into pharmaceutical compositions suitable for administration to a patient in need thereof. Such compositions typically comprise at least one compound of the invention and at least one pharmaceutically acceptable carrier. As used herein the language ‘pharmaceutically acceptable carrier’ is intended to include solid carriers such as lactose, magnesium stearate, terra alba, sucrose, talc, stearic acid, gelatin, agar, pectin, acacia or the like; and liquids such as vegetable oils, arachis oil and sterile water, or the like, any and all solvents, dispersion media, coatings, antibacterial and antifungal agents, isotonic and absorption delaying agents, and the like, compatible with pharmaceutical administration. This listing of pharmaceutically acceptable carriers is not to be construed as limiting. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be appropriate to include isotonic agents, for example, sugars, polyalcohols such as manitol, sorbitol, sodium chloride in the composition. Prolonged absorption of the injectable compositions can be brought about by including in the composition an agent which delays absorption, for example, aluminum mono stearate and gelatin.

Sterile injectable solutions can be prepared by incorporating the active compound (e.g., a compound according to an embodiment of the invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the particular methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

A compound according to an embodiment of the invention may be provided as a salt, particularly as a pharmaceutically acceptable salt of compounds of any of the formulae herein. Examples of pharmaceutically acceptable salts of these compounds include those derived from organic acids such as acetic acid, malic acid, tartaric acid, citric acid, lactic acid, oxalic acid, succinic acid, fumaric acid, maleic acid, benzoic acid, salicylic acid, phenylacetic acid, mandelic acid, methanesulphonic acid, benzenesulphonic acid and p-toluenesulphonic acid, mineral acids such as hydrochloric and sulphuric acid and the like, giving methanesulphonate, benzenesulphonate, p-toluenesulphonate, hydrochloride and sulphate, and the like, respectively or those derived from bases such as organic and inorganic bases. Examples of suitable inorganic bases for the formation of salts of compounds for this invention include the hydroxides, carbonates, and bicarbonates of ammonia, lithium, sodium, calcium, potassium, aluminium, iron, magnesium, zinc and the like. Salts can also be formed with suitable organic bases. Such bases suitable for the formation of pharmaceutically acceptable base addition salts with compounds of the present invention include organic bases which are nontoxic and strong enough to form salts. Such organic bases are already well known in the art and may include amino acids such as arginine and lysine, mono-, di-, or trihydroxyalkylamines such as mono-, di-, and triethanolamine, choline, mono-, di-, and trialkylamines, such as methylamine, dimethylamine, and trimethylamine, guanidine; N-methylglucosamine; N-methylpiperazine; morpholine; ethylenediamine; N-benzylphenethylamine; tris(hydroxymethyl)aminomethane; and the like.

Salts of compounds according to an embodiment of the invention may be prepared in a conventional manner using methods well known in the art. Acid addition salts of said basic compounds may be prepared by dissolving the free base compounds according to the first or second aspects of the invention in aqueous or aqueous alcohol solution or other suitable solvents containing the required acid. Where a compound of the invention contains an acidic function, a base salt of said compound may be prepared by reacting said compound with a suitable base. The acid or base salt may separate directly or can be obtained by concentrating the solution e.g. by evaporation. The compounds of this invention may also exist in solvated or hydrated forms.

The following formulation examples illustrate representative pharmaceutical compositions that may be prepared in accordance with this invention. The present invention, however, is not limited to the following pharmaceutical compositions.

### Formulation 1

### Tablets

A compound of the invention is admixed as a dry powder with a dry gelatin binder in an approximate 1:2 weight ratio. A minor amount of magnesium stearate is added as a lubricant. The mixture is formed into 240-270 mg tablets (80-90 mg of active compound per tablet) in a tablet press.

### Formulation 2

### Capsules

A compound of the invention is admixed as a dry powder with a starch diluent in an approximate 1:1 weight ratio. The mixture is filled into 250 mg capsules (125 mg of active compound per capsule).

### Formulation 3

### Liquid

A compound of the invention (125 mg) may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl:cellulose (11:89, 50 mg) in water. Sodium benzoate (10 mg), flavor, and color are diluted with water and added with stirring. Sufficient water may then added to produce a total volume of 5 mL.

### Formulation 4

### Tablets

A compound of the invention may be admixed as a dry powder with a dry gelatin binder in an approximate 1:2 weight ratio. A minor amount of magnesium stearate is added as a lubricant. The mixture is formed into 450-900 mg tablets (150-300 mg of active compound) in a tablet press.

### Formulation 5

### Injection

A compound of the invention is dissolved or suspended in a buffered sterile saline injectable aqueous medium to a concentration of approximately 5 mg/mL. Formulation 6

### Topical

Stearyl alcohol (250 g) and a white petrolatum (250 g) are melted at about 75° C. and then a mixture of a compound of the invention (50 g) methylparaben (0.25 g), propylparaben (0.15 g), sodium lauryl sulfate (10 g), and propylene glycol (120 g) dissolved in water (about 370 g) is added and the resulting mixture is stirred until it congeals.

### Methods of Treatment

The present invention relates also to a method of treatment or prevention of osteoarthritis, which comprises administering to a subject in need thereof, a therapeutically effective amount of compound of the invention.

The present invention relates also to a method of treatment or prevention of osteoarthritis, which comprises administering to a subject in need thereof, a therapeutically effective amount of an inhibitor of PDE1A according to any of the Formulae herein.

The present invention relates also to a method of treatment or prevention of osteoarthritis, which comprises administering to a subject in need thereof, a therapeutically effective amount of an inhibitor of PDE1 A according to any of the Formulae herein.

Another aspect of the present method invention relates to a method of treatment or prophylaxis of a condition characterized by abnormal PDE1A activity, which comprises administering a therapeutically effective amount of a PDE1A inhibiting compound according to any of the Formulae herein.

A further aspect of the present method invention is a method of treatment or prophylaxis of a disease involving degradation of cartilage, which comprises administering a therapeutically effective a compound according to any of the Formulae herein.

A special embodiment of the present method invention is a method of treatment or prevention of OA, which comprises administering to a subject in need thereof, a therapeutically effective amount of a compound according to any of the Formulae herein.

This invention also relates to the use of the present compounds in the manufacture of a medicament for treatment or prophylaxis of a condition prevented, ameliorated or eliminated by administration of an inhibitor of PDE1A which is a compound of the invention, or a condition selected from diseases involving inflammation, most particularly for the treatment of diseases selected from osteoarthritis and rheumatoid arthritis.

Administration of the compound of the present invention to the subject patient includes both self-administration and administration by another person. The patient may be in need of treatment for an existing disease or medical condition, or may desire prophylactic treatment to prevent or reduce the risk for diseases and medical conditions affected by a disturbance in bone metabolism. The compound of the present invention may be delivered to the subject patient orally, transdermally, via inhalation, injection, nasally, rectally or via a sustained release formulation.

A particular regimen of the present method comprises the administration to a subject in suffering from a disease condition characterized by a disturbance in bone and/or cartilage metabolism, of an effective PDEIA-inhibiting amount of a compound of the present invention for a period of time sufficient to reduce the abnormal levels of bone and/or cartilage degradation in the patient, and particularly terminate, the self-perpetuating processes responsible for said degradation. A special embodiment of the method comprises administering of an effective PDE1A inhibiting amount of a compound of the present invention to a subject patient suffering from or susceptible to the development of osteoarthritis, for a period of time sufficient to reduce or prevent, respectively, collagen and bone degradation in the joints of said patient, and particularly terminate, the self-perpetuating processes responsible for said degradation.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Particular compounds are those that exhibit large therapeutic indices. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies particularly within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

A particular therapeutically effective amount of the compound of the present invention to administer to a subject patient is about 0.1 mg/kg to about 10 mg/kg administered from once to three times a day. For example, an effective regimen of the present method may administer about 5 mg to about 1000 mg of said compound of the present invention from once to three times a day. It will be understood, however, that the specific dose level for any particular subject patient will depend upon a variety of factors including the age, body weight, general health, sex, diet, time of administration, route of administration, rate of excretion, drug combination and the severity of the particular inflammatory condition. A consideration of these factors is well within the purview of the ordinarily skilled clinician for the purpose of determining the therapeutically effective or prophylactically effective dosage amount needed to prevent, counter, or arrest the progress of the condition.

For the prevention and/or treatment of long-term conditions, the regimen for treatment usually stretches over many months or years so oral dosing is appropriate for patient convenience and tolerance. With oral dosing, one to five and especially two to four and typically three oral doses per day are representative regimens. Using these dosing patterns, each dose provides from about 0.01 to about 20 mg/kg of the compound of the invention, with particular doses each providing from about 0.1 to about 10 mg/kg and especially about 1 to about 5 mg/kg.

Transdermal doses are generally selected to provide similar or lower blood levels than are achieved using injection doses.

When used to prevent the onset of a condition related to bone and/or cartilage degradation the compounds of this invention will be administered to a patient at risk for developing the condition, typically on the advice and under the supervision of a physician, at the dosage levels described above. Patients at risk for developing a particular condition generally include those who have been identified by genetic testing or screening to be particularly susceptible to developing said condition.

The compounds of this invention can be administered as the sole active agent or they can be administered in combination with other agents, including other compounds that demonstrate the same or a similar therapeutic activity and that are determined to safe and efficacious for such combined administration.

The present invention will now be described in detail with reference to specific examples of compounds and methods for their production. Within this specification embodiments have been described in a way that enables a clear and concise specification to be written, but it will be appreciated that embodiments may be variously combined or separated without parting from the invention.

### EXAMPLES

### 1. Synthetic Preparation of Compounds of the Invention

The compounds of this invention can be prepared from readily available starting materials using the following general methods and procedures. It will be appreciated that where typical or particular process conditions (i.e., reaction temperatures, times, mole ratios of reactants, solvents, pressures, etc.) are given; however, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The following methods are presented with details as to the preparation of representative compounds that have been listed hereinabove. The compounds of the invention may be prepared from known or commercially available starting materials and reagents by one skilled in the art of organic synthesis.

### General Synthetic Procedures

The pyrazolo[4,3-d]pyrimidinones compounds of this invention can be prepared from readily available starting materials using the following general methods and procedures. It will be appreciated that where typical or particular process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The following representative methods are presented with details as to the preparation of representative pyrazolo[4,3-d]pyrimidinones that have been listed herein above. The compounds of the invention may be prepared from known or commercially available starting materials and reagents by one skilled in the art of organic synthesis.

### Synthesis of Intermediates

### Intermediate 1

### 5-Chloro-1-methyl-3-propyl-1,6-dihydro-pyrazolo[4,3-d]pyrimidin-7-one

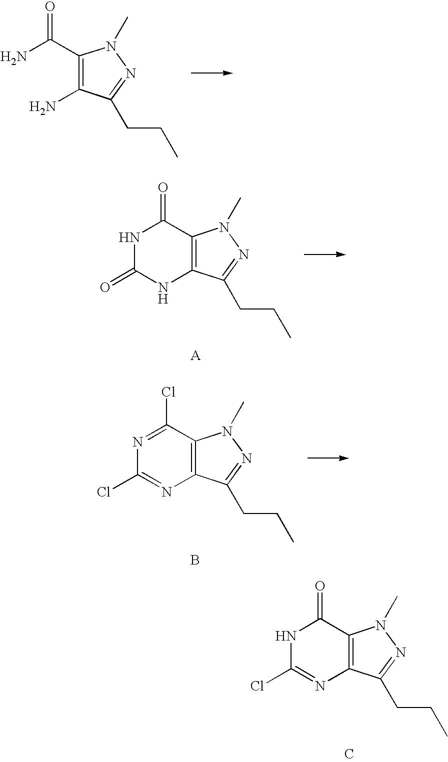

### A. 1-Methyl-3-propyl-1,4-dihydro-pyrazolo[4,3-d]pyrimidine-5,7-dione

4-Amino-1-methyl-3-propyl-pyrazole-5-carboxamide (10.9 mmol) was dissolved in acetonitrile (200 mL) and was heated under reflux. Carbonyldiimidazole (CDI, 13 mmol) was added portionwise while maintaining the temperature. After few minutes a precipitate developed. The solution was kept at reflux for until complete consumption of starting material (16 h). On cooling to room temperature, the suspension was filtered. The resulting solid was dried under reduced pressure. This solid was then used in the next step without further purification. MS (ES+) 209.1 (M+1); HPLC tr=0.85 min. (Column used for all LCMS analysis: Waters Acquity HPLC BEH C18 1.7 μm, 2.1 mm ID×50 mm L (Part No. 186002350)).

### B. 5,7-Dichloro-1-methyl-3-propyl-1H-pyrazolo[4,3-d]pyrimidine

1-Methyl-3-propyl-1,4-dihydro-pyrazolo[4,3-d]pyrimidine-5,7-dione (4.8 mmol) was dissolved in phosphorus oxychloride at room temperature, under nitrogen. A trace of DMF was added to the reaction mixture and the solution was heated under reflux for 7 h-16 h. On completion of the reaction, the excess of phosphorus oxychloride was removed under reduced pressure. The reaction mixture was poured onto ice/water and extracted with ethyl acetate. The organic layer was dried over anhydrous magnesium sulfate. Removal of solvent under reduced pressure afforded a soft solid, which was used without further purification in the next step.

MS (ES+) 245.1 (M+1); 247 (M+3); HPLC tr=1.41 min.

### C. 5-Chloro-1-methyl-3-propyl-1,6-dihydro-pyrazolo[4,3-d]pyrimidin-7-one

Potassium hydroxide (20 mmol) was added to a solution of 5,7-dichloro-1-methyl-3-propyl-1H-pyrazolo[4,3-d]pyrimidine (4 mmol) in dioxane/water (20 mL/5 mL) at room temperature. The solution was heated under reflux for 1 h. After complete reaction, the mixture was allowed to cool to room temperature. The solution was treated with dilute aqueous hydrochloric acid (2M) until pH=1 was achieved and then extracted with ethyl acetate. The organic phase was dried over anhydrous magnesium sulfate and then the solvent removed under reduced pressure to afford 5-Chloro-1-methyl-3-propyl-1,6-dihydro-pyrazolo[4,3-d]pyrimidin-7-one.

MS (ES+) 227.1 (M+1); HPLC tr=1.04 min. NMR (¹H, DMSO) 3.9 (3H, s, CH₃); 2.6 (2H, d, J=7.2 Hz, CH₂); 1.69 (2H, q, J=7.2 Hz, CH₂), 0.91 (3H, t, J=7.2 Hz, CH₃).

### Intermediate 2

### 5-Chloro-1-methyl-3-cyclopentyl-1,6-dihydro-pyrazolo[4,3-d]pyrimidin-7-one

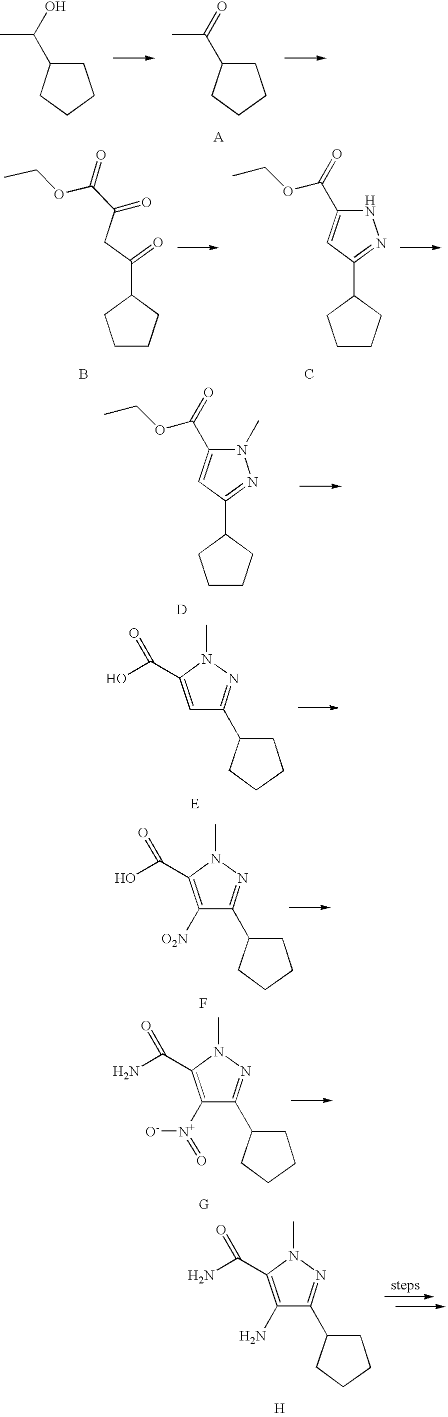

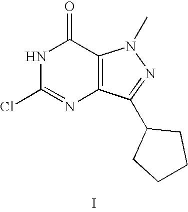

### A. 1-Cyclopentyl ethanone

A solution of chromium(VI) oxide (0.32 mol) in water (50 mL was added to sulfuric acid (28 mL, 0.52 mmol) dropwise at room temperature for 30 min. This solution was added dropwise to 1-cyclopentylethanol (0.21 mol) in acetone (550 mL) at 35° C. until the orange colour persisted for 10 min. The reaction was quenched using iso-propyl alcohol (25 mL) and treated with solid sodium hydrogencarbonate (90 g) until a pH of 5 was obtained. The mixture was filtered and the filtrate was extracted with methyl tert-butyl ether (2×75 mL). The combined methyl tert-butyl ether layer was washed with water (20 mL×3) followed by saturated aqueous sodium chloride (25 mL). The organic phase was dried over anhydrous sodium sulfate, filtered and the solvent removed under reduced pressure to afford 1-cyclopentyl ethanone as a colorless liquid. No further purification was performed, and crude product was used directly in next step.

Yield of the compound: 27 g.

### B. 4-Cyclopentyl-2,4-dioxobutyric acid ethyl ester

To a solution of sodium ethoxide in ethanol (5.54 g of sodium in 300 mL of ethanol) at room temperature, a mixture of 1-cyclopentylethanone (0.241 mol) and diethyloxalate (0.265 mol) in ethanol (150mL) was added dropwise. The temperature was raised to 60° C. and maintained for 2 h. On completion of the reaction, the mixture was poured onto cold, dilute aqueous hydrochloric acid (2M, 320mL) and the product was extracted with ethyl acetate (75 mL×3). The combined organic phases were washed with water (20 mL×3), followed by saturated aqueous sodium chloride (30 mL). The organic phase was dried over anhydrous sodium sulfate, filtered and the solvent removed under reduced pressure. The product was obtained as a dark orange liquid. No further purification was performed, and crude material was used directly in next step.

Weight of the compound: 34.6 g, Yield: 77%.

### C. 5-Cyclopentyl-1H-pyrazole-3-carboxylic acid ethyl ester

Hydrazine hydrate (0.070 mol) was added to a solution of 4-cyclopentyl-2,4-dioxobutyric acid ethyl ester (0.067 mol) in ethanol (50 mL). The reaction mixture was stirred at room temperature for 16 h. On completion of the reaction, ethanol was removed from the mixture under reduced pressure. The crude material was dissolved in dichloromethane (100 mL) and washed with water (25 mL) followed by saturated aqueous sodium chloride solution (30 mL). The organic phase was dried over anhydrous sodium sulfate, filtered and the solvent removed under reduced pressure. The product was obtained as a viscous liquid.

Weight of the compound: 8.5 g Yield: 77%.

MS (ES+): 209 (M+1).

### D. 5-Cyclopentyl-2-methyl-2H-pyrazole-3-carboxylic acid ethyl ester

To a solution of to 5-cyclopentyl-1H-pyrazole-3-carboxylic acid ethyl ester (0.040 mol) in toluene, dimethylsulfate (0.040 mol) was added and the reaction mixture was stirred at 90° C. for 2.5 h. On completion of the reaction, the mixture was diluted with dichloromethane (100 mL) and washed with water (20 mL×3), followed by saturated aqueous sodium carbonate (20 mL) and saturated aqueous sodium chloride solution. The organic phase was dried over sodium sulfate, filtered and the solvent removed under reduced pressure. The product was obtained as a brown liquid.

Weight of the compound: 6.16 g Yield: 68%. MS (ES+): 223 (M+1). NMR (¹H, CDCl₃): 6.4 (1H, s, CH); 4.3 (2H, q, OCH₂); 4.1 (3H, s, NCH₃); 3.1 (1H, m, CH); 2 (2H, m, CH₂); 1.75 (2H, m, CH₂); 1.65 (4H, s, 2×CH₂); 1.35 (3H, t, CH₃).

### E. 5-Cyclopentyl-2-methyl-1H-pyrazole-3-carboxylic acid

Aqueous sodium hydroxide (3.27 g of sodium hydroxide and 14 mL of water) was added to 5-cyclopentyl-2-methyl-1H-pyrazole-3-carboxylic acid ethyl ester (0.027 mol) and heated under reflux for about 2.5 h. On completion of the reaction, the mixture was diluted with water (50 mL) and treated with aqueous hydrochloric acid (6.97 mL, 35%) to precipitate the product. The solid was collected by filtration and dried under reduced pressure to give the product as an off-white solid.

Weight of the compound: 3.6 Yield: 70% MS (ES+): 195 (M+1) M.Pt.: 123-128° C.

### F. 5-Cyclopentyl-2-methyl-4-nitro-1H-pyrazole-3-carboxylic acid

Oleum (4.24mL) was added slowly to fuming nitric acid at 0° C. To this was added portionwise, 5-cyclopentyl-2-methyl-1H-pyrazole-3-carboxylic acid (2 g, 10 mmol) at less than 60° C. and thereafter maintained at 60° C. for 2.5 h. On completion of the reaction the mixture was allowed to cool and poured on to crushed ice to give a precipitate. The solid was collected by filtration and dried under reduced pressure to give the product as pale yellow solid.

Weight of the compound: 1.4 g Yield: 58%. MS (ES+): 240 (M+1). M.Pt: 134-138° C.

### G. 5-Cyclopentyl-2-methyl-4-nitro-2H-pyrazole-3-carboxylic acid amide

Thionyl chloride and 5-cyclopentyl-2-methyl-4-nitro-1H-pyrazole-3-carboxylic acid (3 mmol) were heated under reflux for 3 h. On completion of the reaction, thionyl chloride was distilled out from the reaction mixture and the residue was dissolved in acetone (7 mL) and ice cold aqueous ammonia was added dropwise. The solid was collected by filtration and dried under reduced pressure to give the product as an off-white solid.

Weight of the compound: 830 mg Yield: 59%. MS (ES+): 239 (M+1). M.Pt: 133-135° C.

### H. 4-Amino-5-cyclopentyl-2-methyl-2H-pyrazole-3-carboxylic acid amide

Palladium (10% on charcoal) was added to 5-cyclopentyl-2-methyl-4-nitro-1H-pyrazole-3-carboxamide (4.5 g, 18 mmol) in methanol (50 mL). The reaction mixture was stirred at room temperature for 5 h under hydrogen atmosphere. On completion of the reaction, the catalyst was removed by filtration through a pad of celite and the filtrate was concentrated under reduced pressure. The resulting crude product (4.6 g) was purified by column chromatography eluting initially with hexane followed by 2% of ethyl acetate in hexane. The product was isolated as a light pink solid.

Weight of the compound: 1.6 g Yield: 95%. M.Pt: 116-120° C.

### I. 5-Chloro-1-methyl-3-cyclopentyl-1,6-dihydro-pyrazolo[4,3-d]pyrimidin-7-one

This intermediate was obtained using same reaction sequence as that for 5-chloro-1-methyl-3-propyl-1,6-dihydro-pyrazolo[4,3-d]pyrimidin-7-one starting with 4-amino-3-cyclopentyl-1-methyl-1H-pyrazole-5-carboxamide instead of 4-amino-3-propyl-1-methyl-1H-pyrazole-5-carboxamide MS (ES+): 253 (M+1), 255. ¹H NMR (400 MHz, CDCl₃) 10.39 (1H, brs), 4.23 (3H, s), 3.37 (1H, pent, J=8 Hz), 2.11 (2H, m), 1.91-1.83 (4H, m), 1.69 (2H, m).

### Intermediate 3

### 5-Chloro-1-methyl-3-tert-butyl-1,6-dihydro-pyrazolo[4,3-d]pyrimidin-7-one

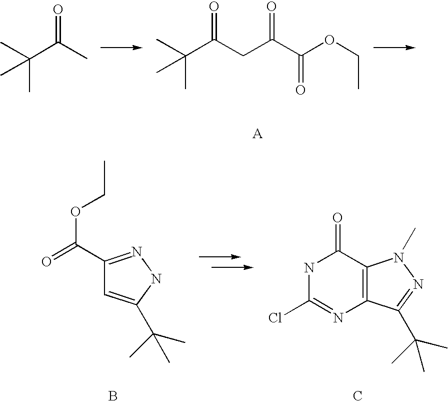

### A. 5,5-Dimethyl-2,4-dioxohexanoic acid ethyl ester

To a solution of sodium ethoxide (740 mmol of sodium in 450 mL ethanol), a mixture of methyl tert-butyl ketone (740 mmol) and diethyl oxalate (820 mmol) were added dropwise at room temperature and stirred for 1 h at 78° C. On completion of the reaction, the mixture was allowed to cool to room temperature and poured onto cold aqueous hydrochloric acid (5M, 800 mL). Following extraction with methyl tert-butyl ether (300 mL×3), the combined organic phases were washed with saturated aqueous sodium chloride (500 mL) and dried over anhydrous sodium sulfate. After filtration, removal of the solvent under reduced pressure afforded the product as a red liquid.

Compound wt: 140 g, Yield: 93.3%. MS (ES+): 199 (M+1). NMR (¹H, CDCl₃): 6.5 (1H, s, CH); 4.3 (2H, q, OCH₂); 1.35 (3H, t, CH₃); 1.15 (9H, s, 3×CH₃).

### B. 5-tert-butyl-1H-pyrazole-3-carboxylic acid ethyl ester

To a solution of methyl 5,5-dimethyl-2,4-dioxohexanoic acid ethyl ester (720 mmol) in ethanol (500 μL) was added dropwise, at 0° C., hydrazine hydrate (640 mmol) and stirred for 3 h at room temperature. On completion of the reaction, the mixture was diluted with water (1 L) and stirred for 10 min. The precipitate was collected by filtration and dried at room temperature for 5 hours. The product was isolated as an off-white solid.

Compound wt: 107 g Yield: 75%. MS (ES−): 195 (M−1). NMR (¹H, CDCl₃): 6.6 (1H, s, CH); 4.3 (2H, q, OCH₂); 1.2 (12H, m, 4×CH₃). M.Pt.: 138-139° C.

### C. 5-Chloro-1-methyl-3-tert-butyl-1,6-dihydro-pyrazolo[4,3-d]pyrimidin-7-one

This intermediate was obtained using same reaction sequence as that for 5-chloro-1-methyl-3-cyclopentyl-1,6-dihydro-pyrazolo[4,3-d]pyrimidin-7-one starting with 5-tert-butyl-1H-pyrazole-3-carboxylic acid ethyl ester instead of 5-cyclopentyl-1H-pyrazole-3-carboxylic acid ethyl ester.

MS (ES+): 223 (M+1). NMR (¹H, CDCl₃): 13.15 (1H, s, NH); 4.08 (3H, s, CH₃); 1.37 (3H, s, CH₃).

### Intermediate 4

### 5-Chloro-1-ethyl-3-tert-butyl-1,6-dihydro-pyrazolo[4,3-d]pyrimidin-7-one

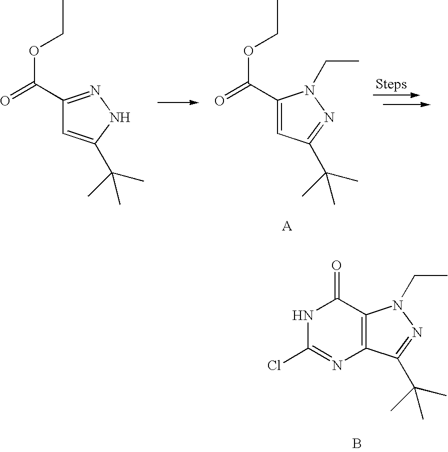

### A. 5-tert-butyl-2-ethyl-pyrazole-3-carboxylic acid ethyl ester

To a suspension of sodium hydride (20 mmol) in dimethylformamide (5 mL) at 0° C. was added a solution of 5-tert-butyl-1H-pyrazole-3-carboxylic acid ethyl ester. The resulting mixture was stirred for 30 min at room temperature before being cooled to 0° C. Ethyl bromide (40 mmol) was added dropwise. The resulting mixture was heated to 55° C. for 18 h. On completion of the reaction, the mixture was cooled to room temperature and diluted with water (50 mL). Extraction of the mixture with methyl tert-butyl ether was performed (50 mL×2). The combined organic phases were washed with saturated aqueous sodium chloride (50 mL) and dried over anhydrous sodium sulfate. Following filtration, the solvent was removed under reduced pressure. Crude compound (2.2 g) was purified by column chromatography using silica gel, eluting with 1% ethyl acetate, hexane. The product was isolated as a yellow liquid.

Compound wt: 1.3 g. Yield: 57%. NMR (¹H, CDCl₃): 6.6 (1H, s, CH); 4.55 (2H, q, CH₂); 4.55 (2H, q, CH₂); 1.2-1.4 (15H, m, 5×CH₃).

### B. 5-Chloro-1-ethyl-3-tert-butyl-1,6-dihydro-pyrazolo[4,3-d]pyrimidin-7-one

This intermediate was obtained using same reaction sequence as that for 5-chloro-1-methyl-3-cyclopentyl-1,6-dihydro-pyrazolo[4,3-d]pyrimidin-7-one, starting with 2-ethyl-5-tert-butyl-pyrazole-3-carboxylic acid ethyl ester instead of 2-methyl-5-cyclopentyl-pyrazole-3-carboxylic acid ethyl ester.

MS (ES+): 255 (M+1), HPLC rt: 1.33 min. NMR (¹H, CDCl₃) 4.46 (2H, q, J 7.2, NCH₂); 1.38 (9H, s, 3×CH₃); 1.35 (2H, t, J 7.2, CH₃).

### Intermediate 5

### 3-bromo-5-dipropylamino-1-methyl-1,6-dihydro-pyrazolo[4,3-d]pyridine-7-one

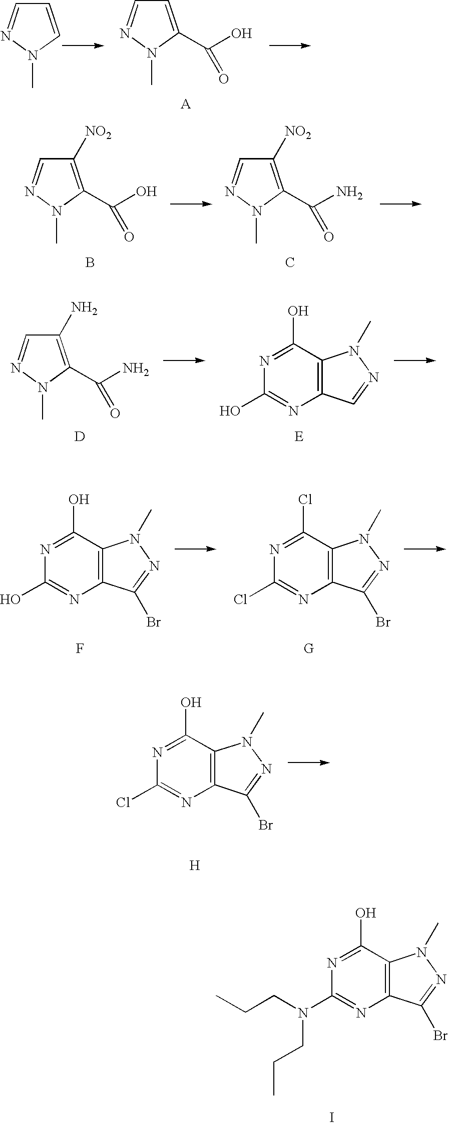

### A. 2-Methyl-2H-pyrazole-3-carboxylic acid

To a stirred solution of 1-methyl-1H-pyrazole (609 mmol) in dry ether (600 mL) under an atmosphere of nitrogen was added a solution of n-BuLi in hexane (2.6M, 670 mmol) dropwise at −78oC over a period of 1 h. The reaction mixture was stirred at this temperature for a further 1 hour, and then dry carbon dioxide gas was passed through the mixture at −78oC for 30 min. The reaction mixture was then allowed to warm to room temperature and quenched with water (500 mL). The aqueous phase was separated, washed with ether (500 mL) and cooled to 2-3oC. To the stirred mixture was added concentrated aqueous hydrochloric acid until a pH of 3 was obtained. The resulting precipitate was collected by filtration, washed with ice-cold water (20 mL), dried first in open air, and then in a vacuum desiccator over phosphorus pentoxide to afford 2-methyl-2H-pyrazole-3-carboxylic acid as a white powder.

Compound wt: 35.3 g, 45.4% yield. ¹H NMR (400 MHz, DMSO-d₆) δ: 13.31(1H, bs); 7.50 (1H, d); 6.81 (1H, d); 4.07 (3H, s).

### B. 2-Methyl-4-nitro-2H-pyrazole-3-carboxylic acid

Oleum (1977 mmol) was slowly added to fuming nitric acid (777 mmol) followed by the addition of 2-methyl-2H-pyrazole-3-carboxylic acid (277 mmol) in small portions maintaining the reaction temperature below 60oC. Stirring at this temperature was continued for a further 1 h. On completion, the reaction mixture was poured onto crushed ice and extracted with ethyl acetate (300 mL×3). The combined organic phases were washed with water (250 mL×2) and dried over anhydrous sodium sulfate. The solvent was removed under reduced pressure to afford 2-methyl-4-nitro-2H-pyrazole-3-carboxylic acid as a light yellow solid.

Compound wt: 23.6 g, 50%. ¹H NMR (400 MHz, DMSO-d₆) δ: 8.29 (1H, s); 3.95 (3H, s).

### C. 2-Methyl-4-nitro-2H-pyrazole-3-carboxylic acid amide

A mixture of 2-methyl-4-nitro-2H-pyrazole-3-carboxylic acid (138.8 mmol) and thionyl chloride (300 mL) was heated under reflux for 12 h. The mixture was concentrated to dryness under reduced pressure. The resulting oil was dissolved in acetone (200 mL) and added to cold aqueous ammonium hydroxide with stirring. The precipitate was collected by filtration and dried to give 2-methyl-4-nitro-2H-pyrazole-3-carboxylic acid amide as an off-white solid.

Compound wt: 10.5 g, 45% yield. ¹H NMR (400 MHz, DMSO-d₆) δ: 8.47 (1H, s); 8.32 (1H, s); 8.27 (1H, s); 3.86 (3H, s).

### D. 4-Amino-2-methyl-2H-pyrazole-3-carboxylic acid amide

To 2-methyl-4-nitro-2H-pyrazole-3-carboxylic acid amide (61.2 mmol) in ethanol (100 mL) was added palladium (1 g, 10% on Charcoal) and the reaction mixture was stirred under hydrogen atmosphere (Parr shaker, 40 PSI) for five hours. On completion, the reaction mixture was filtered through a celite pad and the solvent removed under reduced pressure to give 4-amino-2-methyl-2H-pyrazole-3-carboxylic acid amide as a pale yellow solid.

Compound wt: 6.9 g, 80% yield. M.p. 158-160° C. ¹H NMR (400 MHz, DMSO-d₆) δ: 7.37 (2H, bs); 7.01 (1H, s); 4.44 (2H, bs); 3.88 (3H, s). ¹³C NMR (100 MHz, DMSO-d6) δ: 38.80, 122.9788, 129.1571, 132.5167, 161.8173; Mass (m/z): 141 (M+H)

### E. 1-Methyl-3a,7a-dihydro-1H-pyrazolo[4,3-d]pyrimidine-5,7-diol

To a stirred solution of 4-amino-2-methyl-2H-pyrazole-3-carboxylic acid amide (46.7 mmol) in acetonitrile (100 mL), heated under reflux, was added carbonyldiimidazole (68.6 mmol) over a period of 1 h. The reaction mixture was heated under reflux for a further 24 h. On completion, the reaction mixture was cooled and the precipitated solid was collected by filtration and dried completely to afford 1-methyl-3a,7a-dihydro-1H-pyrazolo[4,3-d]pyrimidine-5,7-diol as a light yellow solid.

Compound wt: 6.2 g, 80% yield. ¹H NMR (400 MHz, DMSO-d6) δ: 11.13 (1H, bs); 10.98 (1H, bs); 7.34 (1H, s); 4.04 (3H, s).

### F. 3-Bromo-1-methyl-3a,7a-dihydro-1H-pyrazolo[4,3-d]pyrimidine-5,7-diol

To a mixture of 1-methyl-3a,7a-dihydro-1H-pyrazolo[4,3-d]pyrimidine-5,7-diol (36.1 mmol) and NBS (43.4 mmol) in acetonitrile (120 mL) was added acetic acid (0.5 mL) and the reaction mixture was heated under reflux for 6 hours. On completion, the reaction mixture was cooled to room temperature. The white precipitate thus obtained was collected by filtration and dried to give 3-bromo-1-methyl-3a,7a-dihydro-1H-pyrazolo[4,3-d]pyrimidine-5,7-diol as a brown solid.

Compound wt: 4.9 g, 55% yield. ¹H NMR (400 MHz, DMSO-d6) δ: 11.10 (1H, bs); 10.95 (1H, bs); 4.03 (3H, s). Mass (m/z): 243 (M−2H), 245 (M−1H).

### G. 3-Bromo-5,7-dichloro-1-methyl-3a,7a-dihydro-1H-pyrazolo[4,3-d]pyrimidine

A solution of 3-bromo-1-methyl-3a,7a-dihydro-1H-pyrazolo[4,3-d]pyrimidine-5,7-diol (18.4 mmol) in phosphorus oxychloride (100 ml) was heated to 120oC for 16 h. On completion, the excess of phosphorus oxychloride was removed under reduced pressure from reaction mixture. The residue was poured onto crushed ice and solid obtained was collected by filtration. After drying, the crude product was purified by column chromatography (60-120 mesh silica gel) eluting with ethyl acetate:hexane (2:8) to afford title 3-bromo-5,7-dichloro-1-methyl-3a,7a-dihydro-1H-pyrazolo[4,3-d]pyrimidine as an off-white solid.

Compound wt: 2.32 g, 45% yield. ¹H NMR (400 MHz, DMSO-d6) δ: 4.31(3H, s). Mass (m/z): 282 (M+H), 284(M+2H).

### H. 3-Bromo-5-chlorol-methyl-3a,7a-dihydro-1H-pyrazolo[4,3-d]pyrimidin-7-ol

To a stirred solution of 3-bromo-5,7-dichloro-1-methyl-3a,7a-dihydro-1H-pyrazolo[4,3-d]pyrimidine (7.83 mmol) in tetrahydrofuran (10 mL) was added to aqueous potassium hydroxide (6M, 15mL) dropwise at 0-5oC. The reaction mixture was stirred at room temperature for 2 h. On completion, the reaction mixture was treated with aqueous hydrochloric acid (2M) until a pH of 2 was obtained. Extraction with ethyl acetate (20 mL×3) was performed and the combined organic phases were washed with saturated aqueous sodium chloride. After being dried over anhydrous magnesium sulfate, the solvent was removed under reduced pressure. The crude compound was purified by washing with ether to afford 3-bromo-5-chloro-1-methyl-3a,7a-dihydro-1H-pyrazolo[4,3-d]pyrimidin-7-ol as a light yellow solid.

Compound wt: 1.85 g, 90% yield. 1H NMR (400 MHz, DMSO-d6) δ: 4.15 (3H, s). ¹³C NMR (100 MHz, DMSO-d₆) δ: 38.6165, 118.1215, 125.8725, 137.0813, 141.3023, 153.2573; Mass (m/z): 263 (M+, 100%), 265 (M+2H, 100). M.p. 260-262° C.

### I. 3-Bromo-5-dipropylamino-1-methyl-3a,7a-dihydro-1H-pyrazolo[4,3-d]pyrimidin-7-ol

A mixture of 3-bromo-5-chloro-1-methyl-3a,7a-dihydro-1H-pyrazolo[4,3-d]pyrimidin-7-ol (6.46 mmol), N,N′-dipropyl amine (7.75 mmol), and Hunigs base (1.75 mL) in 20mL of t-BuOH (20 mL) was heated under reflux for 3 days. On completion, the reaction mixture was cooled to room temperature. The white solid obtained was collected by filtration and dried to afford 3-bromo-5-dipropylamino-1-methyl-3a,7a-dihydro-1H-pyrazolo[4,3-d]pyrimidin-7-ol as a light yellow solid.

Compound wt: 1.6 g, 75% yield. ¹H NMR (400 MHz, DMSO-d₆) δ: 11.09 (1H, s); 4.04 (3H, s); 3.41 (4H, t); 1.50-1.55 (4H, m); 0.85 (6H, t). ¹³C NMR (100 MHz, DMSO-d₆) δ: 10.9120, 20.5125, 38.3307, 49.2171, 115.7339, 121.2220, 139.9948, 150.3785, 154.5420. Mass (m/z): 328 (M+, 100%), 330 (M+2H, 100%). M.p. 186-187° C.

### Representative Methods

### Method A

The corresponding amine (0.26 mmol) was added to a solution of corresponding Cl-derivative (intermediates 1 to 6) (8.84×10⁻² mmol) in t-BuOH (0.5 mL). The reaction was heated in a sealed tube to 100° C. to 120° C. for 12-72 h. After complete reaction (monitored by LCMS), the mixture was allowed to cool to room temperature. The solvent was removed under reduced pressure to give the crude product. The final compound was isolated by preparative HPLC (yield 10-50%).

Analytical: Waters Acquity HPLC BEH C18 1.7 μm, 2.1 mm ID×50 mm L (Part No. 186002350). Preparative HPLC: Waters XBridge Prep C18 5 μm ODB 19 mm ID×100 mm L (Part No. 186002978). All the methods used MeCN/H₂O gradients. H₂O contained either 0.1% Trifluoroacetic acid (TFA) or 0.1% Ammonia.

### Method B

To the corresponding primary amine (4.8 mmol) in THF (25 mL) at room temperature were added aldehyde (5.6 mmol) and anhydrous magnesium sulfate (0.75 g). After stirring for 1.5 h at room temperature, sodium borohydride (4.8 mmol) was added and the mixture stirred for a further 2 h. Water (10-15 mL) was added to the mixture and stirring resumed for 30-60 min. Additional water was added (20-30 mL) and the mixture was extracted with CH₂Cl₂ (40 mL×3). After being dried over anhydrous magnesium sulfate the solvent was removed under reduced pressure to give the crude product. This material was used in subsequent steps without further purification.

The amine obtained in the previous step (0.26 mmol) was added to a solution of corresponding Chloro-derivative (8.84×10⁻² mmol) in t-BuOH (0.5 ml). The reaction was heated in a sealed tube tot 100° C. for 12-72 h. On completion of the reaction (monitored by LCMS), the mixture was allowed to cool to room temperature and the solvent was removed under reduced pressure. The final compound was isolated by preparative HPLC (yield 10-50%).

Analytical: Waters Acquity HPLC BEH C18 1.7 μm, 2.1 mm ID×50 mm L (Part No. 186002350). Preparative HPLC: Waters XBridge Prep C18 5 μm ODB 19 mm ID×100 mm L (Part No. 186002978). All the methods used MeCN/H₂O gradients. H₂O contained either 0.1% Trifluoroacetic acid (TFA) or 0.1% Ammonia.

### Method C

In a homogeneous solution of Pd(dppf)Cl₂ (0.012 mmol) and 3-bromo-5-dipropylamino-1-methyl-1,6-dihydro-pyrazolo[4,3-d]pyridine-7-one (0.122 mmol), 1,4-dioxane (0.8 ml) and water (0.4 ml) was added potassium carbonate (0.244 mmol) and boronic acid (0.244 mmol). The reaction was heated to 120° C. under microwave irradiation for 40 min. On cooling to room temperature, the solvents were removed under reduced pressure. The crude product was further purified by preparative HPLC.

Preparative HPLC: Waters XBridge Prep C18 5 μm ODB 19 mm ID×100 mm L. All the methods used MeCN/H₂O gradients. H₂O contained either 0.1% Trifluoroacetic acid (TFA) or 0.1% Ammonia. Microwave: Explorer PSL, CEM discover Nr. 020621.

### Example 1

### 5-(4-(2-Chloro-6-fluorobenzyl)-1,4-diazepan-1-yl)-1-methyl-3-propyl-1H-pyrazolo[4,3-d]pyrimidin-7(6H)-one

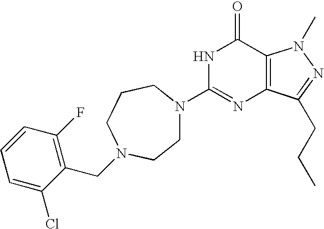

1-(2,6-Difluoro-benzyl)-[1,4]diazepane (0.26 mmol) was added to a solution of 5-chloro-1-methyl-3-propyl-1,6-dihydro-pyrazolo[4,3-d]pyrimidin-7-one (8.84×10⁻² mmol) in t-BuOH (0.5 mL). The reaction was heated in a sealed tube to 100° C. for 24 h. On completion of the reaction (monitored by LCMS), the mixture was allowed to cool to room temperature and the solvent was removed under reduced pressure. The final compound was purified by preparative HPLC.

Preparative HPLC: Waters XBridge Prep C18 5 μm ODB 19 mm ID×100 mm L. The method used MeCN/H₂O 20-45% gradients. H₂O contained 0.1% trifluoroacetic acid (TFA).

### Example 8

### 5-(3,4-dihydroisoquinolin-2(1H)-yl)-1-methyl-3-propyl-1H-pyrazolo[4,3-d]pyrimidin-7(6H)-one

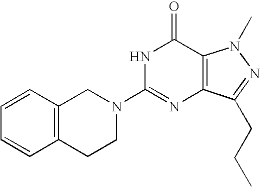

1,2,3,4-Tetrahydro-isoquinoline (0.26 mmol) was added to a solution of 5-chloro-1-methyl-3-propyl-1,6-dihydro-pyrazolo[4,3-d]pyrimidin-7-one (8.84×10⁻² mmol) in t-BuOH (0.5 mL). The reaction was heated in a sealed tube to 100° C. for 24 h. On completion of the reaction (monitored by LCMS), the mixture was allowed to cool to room temperature and the solvent was removed under reduced pressure. The final compound was purified by preparative HPLC.

Preparative HPLC: Waters XBridge Prep C18 5 m ODB 19 mm ID×100 mm L. The method used MeCN/H₂O 35-60% gradients. H₂O contained 0.1% trifluoroacetic acid (TFA).

### Example 25

### 1-methyl-5-(methyl((5-phenylisoxazol-3-yl)methyl)amino)-3-propyl-1H-pyrazolo[4,3-d]pyrimidin-7(6H)-one

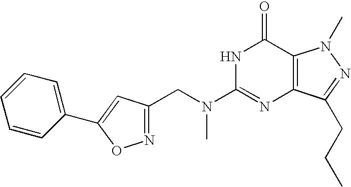

Methyl-(5-phenyl-isoxazol-3-ylmethyl)-amine (0.26 mmol) was added to a solution of 5-chloro-1-methyl-3-propyl-1,6-dihydro-pyrazolo[4,3-d]pyrimidin-7-one (8.84×10−2 mmol) in t-BuOH (0.5 mL). The reaction was heated in a sealed tube to 100° C. for 24 h. On completion of the reaction (monitored by LCMS), the mixture was allowed to cool to room temperature and the solvent was removed under reduced pressure. The final compound was purified by preparative HPLC.

Preparative HPLC: Waters XBridge Prep C18 5 μm ODB 19 mm ID×100 mm L. The method used MeCN/H₂O 45-70% gradients. H₂O contained 0.1% trifluoroacetic acid (TFA).

### Example 37

### 5-(6,7-dimethoxy-1-methyl-3,4-dihydroisoquinolin-2(1H)-yl)-1-methyl-3-propyl-1H-pyrazolo[4,3-d]pyrimidin-7(6H)-one

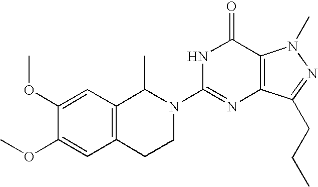

6,7-Dimethoxy-1-methyl-1,2,3,4-tetrahydro-isoquinoline (0.26 mmol) was added to a solution of 5-chloro-1-methyl-3-propyl-1,6-dihydro-pyrazolo[4,3-d]pyrimidin-7-one (8.84×10⁻² mmol) in t-BuOH (0.5 mL). The reaction was heated in a sealed tube to 100° C. for 24 h. On completion of the reaction (monitored by LCMS), the mixture was allowed to cool to room temperature and the solvent was removed under reduced pressure. The final compound was purified by preparative HPLC.

Preparative HPLC: Waters XBridge Prep C18 5 μm ODB 19 mm ID×100 mm L. The method used MeCN/H₂O 35-60% gradients. H₂O contained 0.1% trifluoroacetic acid (TFA).

### Example 45

### 5-((3,4-dichlorobenzyl)(isopentyl)amino)-1-methyl-3-propyl-1H-pyrazolo[4,3-d]pyrimidin-7(6H)-one

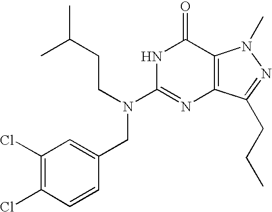

To 3,4-dichloro-benzylamine (0.5 mmol) in THF (2 mL) at room temperature were added 3-methyl-butyraldehyde (0.6 mmol) and anhydrous magnesium sulfate (60 mg). After stirring for 1.5 h at room temperature, sodium borohydride (0.5 mmol) was added and the mixture stirred for a further 2 h. Water (3 mL) was added to the mixture and stirring resumed for 10 min. Additional water was added (1 mL) and the mixture was extracted with CH₂Cl₂ (40 mL×3). After being dried over anhydrous magnesium sulfate the solvent was removed under reduced pressure to give the crude product. This material was used in subsequent steps without further purification.

The amine obtained in the previous step (0.26 mmol) was added to a solution of corresponding 5-chloro-1-methyl-3-propyl-1,6-dihydro-pyrazolo[4,3-d]pyrimidin-7-one (8.84×10⁻² mmol) in t-BuOH (0.5 mL). The reaction was heated in a sealed tube at 100° C. for 12-72 h. On completion of the reaction (monitored by LCMS), the mixture was allowed to cool to room temperature and the solvent was removed under reduced pressure. The final compound was isolated by preparative HPLC.

Preparative HPLC: Waters XBridge Prep C18 5 μm ODB 19 mm ID×100 mm L. The method used MeCN/H₂O 75-100% gradients. H₂O contained 0.1% trifluoroacetic acid (TFA).

### Example 47

### 1′-methyl-5-(methyl((4-methyl-2-phenylthiazol-5-yl)methyl)amino)-3-propyl-1H-pyrazolo[4,3-d]pyrimidin-7(6H)-one

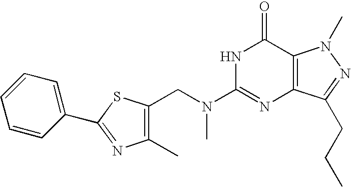

Methyl-(4-methyl-2-phenyl-thiazol-5-ylmethyl)-amine (0.26 mmol) was added to a solution of 5-chloro-1-methyl-3-propyl-1,6-dihydro-pyrazolo[4,3-d]pyrimidin-7-one (8.84×10⁻² mmol) in t-BuOH (0.5 mL). The reaction was heated in a sealed tube to 100° C. for 24 h. On completion of the reaction (monitored by LCMS), the mixture was allowed to cool to room temperature and the solvent was removed under reduced pressure. The final compound was purified by preparative HPLC.

Preparative HPLC: Waters XBridge Prep C18 5 μm ODB 19 mm ID×100 mm L. The method used MeCN/H₂O 45-70% gradients. H₂O contained 0.1% trifluoroacetic acid (TFA).

### Example 52

### 1-methyl-5-((5-methyl-2-phenyl-2H-1,2,3-triazol-4-yl)methylamino)-3-propyl-1H-pyrazolo[4,3-d]pyrimidin-7(6H)-one

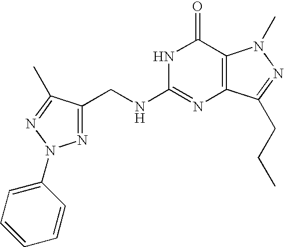

C-(5-Methyl-2-phenyl-2H-[1,2,3]triazol-4-yl)-methylamine (0.26 mmol) was added to a solution of 5-chloro-1-methyl-3-propyl-1,6-dihydro-pyrazolo[4,3-d]pyrimidin-7-one (8.84×10⁻² nmol) in t-BuOH (0.5 mL). The reaction was heated in a sealed tube to 100° C. for 24 h. On completion of the reaction (monitored by LCMS), the mixture was allowed to cool to room temperature and the solvent was removed under reduced pressure. The final compound was purified by preparative HPLC.

Preparative HPLC: Waters XBridge Prep C18 5 μm ODB 19 mm ID×100 mm L. The method used MeCN/H₂O 45-70% gradients. H₂O contained 0.1% trifluoroacetic acid (TFA).

### Example 59

### 5-(4-(2,2-diphenylacetoyl)piperazin-1-yl)-1-methyl-3-propyl-1H-pyrazolo[4,3-d]pyrimidin-7(6H)-one

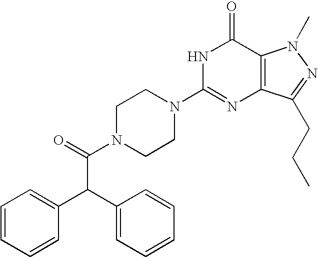

2,2-Diphenyl-1-piperazin-1-yl-ethanone (0.26 mmol) was added to a solution of 5-chloro-1-methyl-3-propyl-1,6-dihydro-pyrazolo[4,3-d]pyrimidin-7-one (8.84×10⁻² mmol) in t-BuOH (0.5 mL). The reaction was heated in a sealed tube to 100° C. for 24 h. On completion of the reaction (monitored by LCMS), the mixture was allowed to cool to room temperature and the solvent was removed under reduced pressure. The final compound was purified by preparative HPLC.

Preparative HPLC: Waters XBridge Prep C18 5 μm ODB 19 mm ID×100 mm L. The method used MeCN/H₂O 45-70% gradients. H₂O contained 0.1% trifluoroacetic acid (TFA).

### Example 70

### 3-cyclopentyl-5-((4-fluorobenzyl)(methyl)amino)-1-methyl-1H-pyrazolo[4,3-d]pyrimidin-7(6H)-one

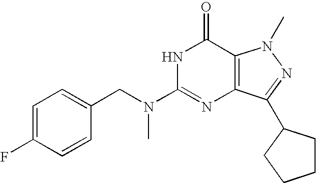

(4-Fluoro-benzyl)-methyl-amine (0.26 mmol) was added to a solution of 5-chloro-1-methyl-3-cyclopentyl-1,6-dihydro-pyrazolo[4,3-d]pyrimidin-7-one (8.84×10⁻² mmol) in t-BuOH (0.5 mL). The reaction was heated in a sealed tube to 100° C. for 24 h. On completion of the reaction (monitored by LCMS), the mixture was allowed to cool to room temperature and the solvent was removed under reduced pressure. The crude product was purified by gradient column chromatography, eluting with 0-5% methanol in dichloromethane, to give the title compound.

### Example 76

### 3-cyclopentyl-5-(3,4-dichlorobenzylamino)-1-methyl-1H-pyrazolo[4,3-d]pyrimidin-7(6H)-one

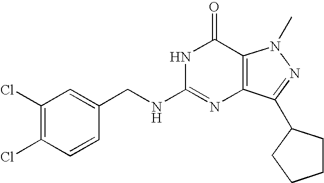

3,4-Dichloro-benzylamine (0.26 mmol) was added to a solution of 5-chloro-1-methyl-3-cyclopentyl-1,6-dihydro-pyrazolo[4,3-d]pyrimidin-7-one (8.84×10⁻² mmol) in t-BuOH (0.5 mL). The reaction was heated in a sealed tube at 100° C. for 24 h. On completion of the reaction (monitored by LCMS), the mixture was allowed to cool to room temperature and the solvent was removed under reduced pressure. The crude product was purified by gradient column chromatography, eluting with 0-5% methanol in dichloromethane, to give the title compound.

### Example 80

### 3-tert-butyl-1-methyl-5-(methyl((4-methyl-2-phenylthiazol-5-yl)methyl)amino)-1H-pyrazolo[4,3-d]pyrimidin-7(6H)-one

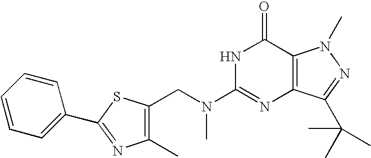

Methyl-(4-methyl-2-phenyl-thiazol-5-ylmethyl)-amine (0.26 mmol) was added to a solution of 5-chloro-1-methyl-3-tert-butyl-1,6-dihydro-pyrazolo[4,3-d]pyrimidin-7-one (8.84×10⁻² mmol) in t-BuOH (1 mL). The reaction was heated in a sealed tube to 100° C. for 24 h. On completion of the reaction (monitored by LCMS), the mixture was allowed to cool to room temperature and the solvent was removed under reduced pressure. The final compound was purified by preparative HPLC.

Preparative HPLC: Waters XBridge Prep C18 5 μm ODB 19 mm ID×100 mm L. The method used MeCN/H₂O 65-90% gradients. H₂O contained 0.1% trifluoroacetic acid (TFA).

### Example 81

### 3-tert-butyl-1-methyl-5-(methyl((1-methyl-3-phenyl-1H-pyrazol-5-yl)methyl)amino)-1H-pyrazolo[4,3-d]pyrimidin-7(6H)-one

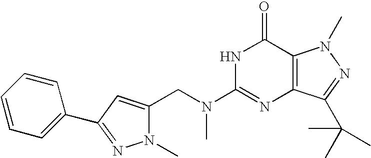

Methyl-(2-methyl-5-phenyl-2H-pyrazol-3-ylmethyl)-amine (0.26 mmol) was added to a solution of 5-chloro-1-methyl-3-tert-butyl-1,6-dihydro-pyrazolo[4,3-d]pyrimidin-7-one (8.84×10⁻² mmol) in t-BuOH (1 mL). The reaction was heated in a sealed tube to 120° C. for 24 h. On completion of the reaction (monitored by LCMS), the mixture was allowed to cool to room temperature and the solvent was removed under reduced pressure. The final compound was purified by preparative HPLC.

Preparative HPLC: Waters XBridge Prep C18 5 μm ODB 19 mm ID×100 mm L. The method used MeCN/H₂O 65-90% gradients. H₂O contained 0.1% trifluoroacetic acid (TFA).

### Example 85

### 3-cyclopentyl-1-methyl-5-(piperidin-1-yl)-1H-pyrazolo[4,3-d]pyrimidin-7(6H)-one

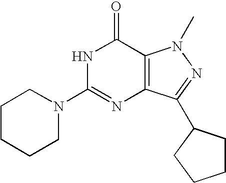

Cyclohexylamine (0.26 mmol) was added to a solution of 5-chloro-1-methyl-3-cyclopentyl-1,6-dihydro-pyrazolo[4,3-d]pyrimidin-7-one (8.84×10⁻² mmol) in t-BuOH (0.5 mL). The reaction was heated in a sealed tube to 100° C. for 24 h. On completion of the reaction (monitored by LCMS), the mixture was allowed to cool to room temperature and the solvent was removed under reduced pressure. The crude product was purified by gradient column chromatography, eluting with 0-5% methanol in dichloromethane, to give the title compound.

### Example 86

### 3-cyclopentyl-1-methyl-5-(2-phenylpyrrolidin-1-yl)-1H-pyrazolo[4,3-d]pyrimidin-7(6H)-one

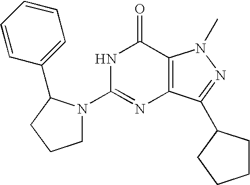

2-Phenyl-pyrrolidine (0.26 mmol) was added to a solution of 5-chloro-1-methyl-3-cyclopentyl-1,6-dihydro-pyrazolo[4,3-d]pyrimidin-7-one (8.84×10⁻² mmol) in t-BuOH (0.5 mL). The reaction was heated in a sealed tube to 100° C. for 24 h. On completion of the reaction (monitored by LCMS), the mixture was allowed to cool to room temperature and the solvent was removed under reduced pressure. The crude product was purified by gradient column chromatography, eluting with 0-5% methanol in dichloromethane, to give the title compound.

### Example 89

### 3-tert-butyl-5-(dipropylamino)-1-methyl-1H-pyrazolo[4,3-d]pyrimidin-7(6H)-one

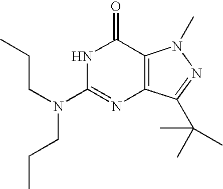

Bispropylamine (0.26 mmol) was added to a solution of 5-chloro-1-methyl-3-tert-butyl-1,6-dihydro-pyrazolo[4,3-d]pyrimidin-7-one (8.84×10−2 mmol) in t-BuOH (1 mL). The reaction was heated in a sealed tube to 120° C. for 24 h. On completion of the reaction (monitored by LCMS), the mixture was allowed to cool to room temperature and the solvent was removed under reduced pressure. The final compound was purified by preparative HPLC.

Preparative HPLC: Waters XBridge Prep C18 5 μm ODB 19 mm ID×100 mm L. The method used MeCN/H₂O 65-90% gradients. H₂O contained 0.1% trifluoroacetic acid (TFA).

### Example 90

### 3-tert-butyl-1-methyl-5-(methyl(3-(2-methylthiazol-4-yl)benzyl)amino)-1H-pyrazolo[4,3-d]pyrimidin-7(6H)-one

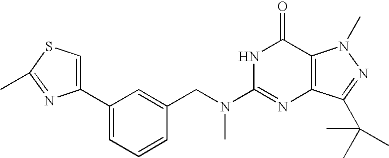

Methyl-[3-(2-methyl-thiazol-4-yl)-benzyl]-amine (0.26 mmol) was added to a solution of 5-chloro-1-methyl-3-tert-butyl-1,6-dihydro-pyrazolo[4,3-d]pyrimidin-7-one (8.84×10−2 mmol) in t-BuOH (1 mL). The reaction was heated in a sealed tube to 120° C. for 24 h. On completion of the reaction (monitored by LCMS), the mixture was allowed to cool to room temperature and the solvent was removed under reduced pressure. The final compound was purified by preparative HPLC.

Preparative HPLC: Waters XBridge Prep C18 5 μm ODB 19 mm ID×100 mm L. The method used MeCN/H₂O 65-90% gradients. H₂O contained 0.1% trifluoroacetic acid (TFA).

### Example 94

### 5-((1H-pyrazol-m-yl)benzyl)(isobutyl)amino)-1-methyl-3-propyl-1H-pyrazolo[4,3-d]pyrimidin-7(6H)-one

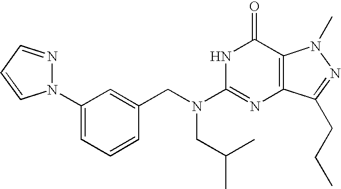

To 3-pyrazol-1-yl-benzylamine (0.5 mmol) in THF (2 mL) at room temperature were added Iso-butylaldehyde (0.6 mmol) and anhydrous magnesium sulfate (60 mg). After stirring for 1.5 h at room temperature, sodium borohydride (0.5 mmol) was added and the mixture stirred for a further 2 h. Water (3 ml) was added to the mixture and stirring resumed for 10 min. Additional water was added (1 ml) and the mixture was extracted with CH2Cl2 (10 mL×3). After being dried over anhydrous magnesium sulfate the solvent was removed under reduced pressure to give the crude product. This material was used in subsequent steps without further purification.

The amine obtained in the previous step (0.26 mmol) was added to a solution of 5-chloro-1-methyl-3-propyl-1,6-dihydro-pyrazolo[4,3-d]pyrimidin-7-one (0.13 mmol) in t-BuOH (0.5 mL). The reaction was heated in a sealed tube tot 100° C. for 12-72 h. On completion of the reaction (monitored by LCMS), the mixture was allowed to cool to room temperature and the solvent was removed under reduced pressure. The final compound was isolated by preparative HPLC.

Preparative HPLC: Waters XBridge Prep C18 5 μm ODB 19 mm ID×100 mm L. The method used MeCN/H₂O 55-80% gradients. H₂O contained 0.1% trifluoroacetic acid (TFA).

### Example 100

### 5-((1H-pyrrol-1-yl)benzyl)(isobutyl)amino)-3-tert-butyl-1-methyl-1H-pyrazolo[4,3-d]pyrimidin-7(6H)-one

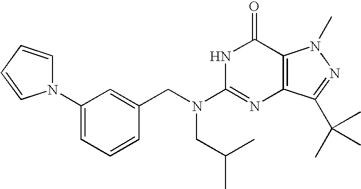

To pyrrol-1-yl-benzylamine (0.5 mmol) in THF (2 mL) at room temperature were added Iso-butylaldehyde (0.6 mmol)) and anhydrous magnesium sulfate (60 mg). After stirring for 1.5 h at room temperature, sodium borohydride (0.5 mmol) was added and the mixture stirred for a further 2 h. Water (3 mL) was added to the mixture and stirring resumed for 10 min. Additional water was added (1 mL) and the mixture was extracted with CH₂Cl₂ (10 ml×3). After being dried over anhydrous magnesium sulfate the solvent was removed under reduced pressure to give the crude product. This material was used in subsequent steps without further purification.

The amine obtained in the previous step (0.26 mmol) was added to a solution of 5-chloro-1-methyl-3-tert-butyl-1,6-dihydro-pyrazolo[4,3-d]pyrimidin-7-one (0.13 mmol) in t-BuOH (0.5 ml). The reaction was heated in a sealed tube to 120° C. for 48 h. On completion of the reaction (monitored by LCMS), the mixture was allowed to cool to room temperature and the solvent was removed under reduced pressure. The final compound was isolated by preparative HPLC.

Preparative HPLC: Waters XBridge Prep C18 5 μm ODB 19 mm ID×100 nm L. The method used MeCN/H₂O 75-100% gradients. H₂O contained 0.1% trifluoroacetic acid (TFA).

### Example 102

### 5-(isobutyl(3-(4-methylpiperazin-1-yl)benzyl)amino)-methyl-3-propyl-1H-pyrazolo[4,3-d]pyrimidin-7(6H)-one

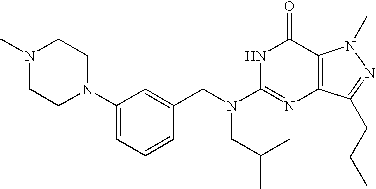

To 3-(4-methyl-piperazin-1-yl)-benzylamine (0.5 mmol) in THF (2 mL) at room temperature were added Iso-butylaldehyde (0.6 mmol) and anhydrous magnesium sulfate (60 mg). After stirring for 1.5 h at room temperature, sodium borohydride (0.5 mmol) was added and the mixture stirred for a further 2 h. Water (3 mL) was added to the mixture and stirring resumed for 10 min. Additional water was added (1 mL) and the mixture was extracted with CH₂Cl₂ (10 mL×3). After being dried over anhydrous magnesium sulfate the solvent was removed under reduced pressure to give the crude product. This material was used in subsequent steps without further purification.

The amine obtained in the previous step (0.26 mmol) was added to a solution of 5-chloro-1-methyl-3-propyl-1,6-dihydro-pyrazolo[4,3-d]pyrimidin-7-one (0.13 mmol) in t-BuOH (0.5 mL). The reaction was heated in a sealed tube to 120° C. for 48 h. On completion of the reaction (monitored by LCMS), the mixture was allowed to cool to room temperature and the solvent was removed under reduced pressure. The final compound was isolated by preparative HPLC.

Preparative HPLC: Waters XBridge Prep C18 5 μm ODB 19 mm ID×100 mm L. The method used MeCN/H₂O 30-55% gradients. H₂O contained 0.1% trifluoroacetic acid (TFA).

### Example 103

### 3-tert-butyl-5-((3,3-dimethylbutyl)(3-morpholinobenzyl)amino)-1-methyl-1H-pyrazolo[4,3-d]pyrimidin-7(6H)-one

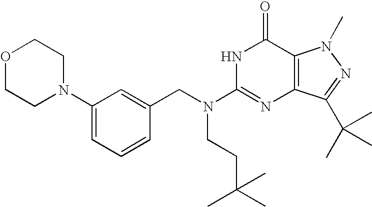

To 3-morpholin-4-yl-benzylamine (0.5 mmol) in THF (2 mL) at room temperature were added 3,3-bismethylbutyraldehyde (0.6 mmol) and anhydrous magnesium sulfate (60 mg). After stirring for 1.5 h at room temperature, sodium borohydride (0.5 mmol) was added and the mixture stirred for a further 2 h. Water (3 mL) was added to the mixture and stirring resumed for 10 min. Additional water was added (1 mL) and the mixture was extracted with CH₂Cl₂ (10 mL×3). After being dried over anhydrous magnesium sulfate the solvent was removed under reduced pressure to give the crude product. This material was used in subsequent steps without further purification.

The amine obtained in the previous step (0.26 mmol) was added to a solution of 5-chloro-1-methyl-3-tert-butyl-1,6-dihydro-pyrazolo[4,3-d]pyrimidin-7-one (0.13 mmol) in t-BuOH (0.5 mL). The reaction was heated in a sealed tube to 120° C. for 48 h. On completion of the reaction (monitored by LCMS), the mixture was allowed to cool to room temperature and the solvent was removed under reduced pressure. The final compound was isolated by preparative HPLC.

Preparative HPLC: Waters XBridge Prep C18 5 μm ODB 19 mm ID×100 mL. The method used MeCN/H₂O 75-100% gradients. H₂O contained 0.1% trifluoroacetic acid (TFA).

### Example 108

### 5-((3,3-dimethylbutyl)(3-morpholinobenzyl)amino)-1-methyl-3-propyl-1H-pyrazolo[4,3-d]pyrimidin-7(6H)-one

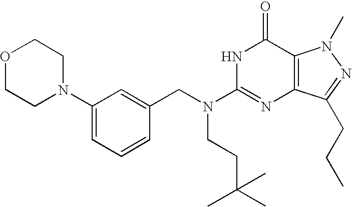

To 3-morpholin-4-yl-benzylamine (0.5 mmol) in THF (2 mL) at room temperature were added 3,3-bismethylbutyraldehyde (0.6 mmol) and anhydrous magnesium sulfate (60 mg). After stirring for 1.5 h at room temperature, sodium borohydride (0.5 mmol) was added and the mixture stirred for a further 2 h. Water (3 mL) was added to the mixture and stirring resumed for 10 min. Additional water was added (1 mL) and the mixture was extracted with CH2Cl2 (10 mL×3). After being dried over anhydrous magnesium sulfate the solvent was removed under reduced pressure to give the crude product. This material was used in subsequent steps without further purification.

The amine obtained in the previous step (0.26 mmol) was added to a solution of 5-chloro-1-methyl-3-propyl-1,6-dihydro-pyrazolo[4,3-d]pyrimidin-7-one (0.13 mmol) in t-BuOH (0.5 mL). The reaction was heated in a sealed tube to 120° C. for 48 h. On completion of the reaction (monitored by LCMS), the mixture was allowed to cool to room temperature and the solvent was removed under reduced pressure. The final compound was isolated by preparative HPLC.

Preparative HPLC: Waters XBridge Prep C18 5 μm ODB 19 mm ID×100 mm L. The method used MeCN/H₂O 65-90% gradients. H₂O contained 0.1% trifluoroacetic acid (TFA).

### Example 116

### 5-((1H-1,2,4-triazol-1-yl)benzyl)(2-methylbutyl)amino)-1-methyl-3-propyl-1H-pyrazolo[4,3-d]pyrimidin-7(6H)-one

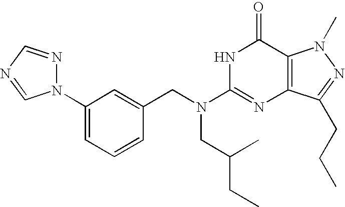

To 3-(1,2,4-triazol-1-yl)-benzylamine (0.5 mmol) in THF (2 mL) at room temperature were added Rac-2-methylbutylaldehyde (0.6 mmol) and anhydrous magnesium sulfate (60 mg). After stirring for 1.5 h at room temperature, sodium borohydride (0.5 mmol) was added and the mixture stirred for a further 2 h. Water (3 mL) was added to the mixture and stirring resumed for 10 min. Additional water was added (1 mL) and the mixture was extracted with CH2Cl2 (10 mL×3). After being dried over anhydrous magnesium sulfate the solvent was removed under reduced pressure to give the crude product. This material was used in subsequent steps without further purification.

The amine obtained in the previous step (0.26 mmol) was added to a solution of 5-chloro-1-methyl-3-propyl-1,6-dihydro-pyrazolo[4,3-d]pyrimidin-7-one (0.13 mmol) in t-BuOH (0.5 mL). The reaction was heated in a sealed tube to 100° C. for 24 h. On completion of the reaction (monitored by LCMS), the mixture was allowed to cool to room temperature and the solvent was removed under reduced pressure. The final compound was isolated by preparative HPLC.

Preparative HPLC: Waters XBridge Prep C18 5 μm ODB 19 mm ID×100 mm L. The method used MeCN/H₂O 45-70% gradients. H₂O contained 0.1% trifluoroacetic acid (TFA).

### Example 118

### 5-((1H-1,2,4-triazol-1-yl)benzyl)(2-methylbutyl)amino)-3-tert-butyl-1-methyl-1H-pyrazolo[4,3-d]pyrimidin-7(6H)-one

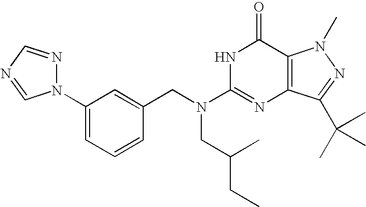

To 3-(1,2,4-triazol-1-yl)-benzylamine (0.5 mmol) in THF (2 mL) at room temperature were added Rac-2-methylbutylaldehyde (0.6 mmol) and anhydrous magnesium sulfate (60 mg). After stirring for 1.5 h at room temperature, sodium borohydride (0.5 mmol) was added and the mixture stirred for a further 2 h. Water (3 mL) was added to the mixture and stirring resumed for 10 min. Additional water was added (1 mL) and the mixture was extracted with CH₂Cl₂ (10 mL×3). After being dried over anhydrous magnesium sulfate the solvent was removed under reduced pressure to give the crude product. This material was used in subsequent steps without further purification.

The amine obtained in the previous step (0.26 mmol) was added to a solution of 5-chloro-1-methyl-3-tert-butyl-1,6-dihydro-pyrazolo[4,3-d]pyrimidin-7-one (0.1 3 mmol) in t-BuOH (0.5 mL). The reaction was heated in a sealed tube tot 100° C. for 24 h. On completion of the reaction (monitored by LCMS), the mixture was allowed to cool to room temperature and the solvent was removed under reduced pressure. The final compound was isolated by preparative HPLC.

Preparative HPLC: Waters XBridge Prep C18 5 μm ODB 19 mm ID×100 mm L. The method used MeCN/H₂O 65-90% gradients. H₂O contained 0.1% trifluoroacetic acid (TFA).

### Example 124

### 3-tert-butyl-5-((2-methylpropyl)(3-morpholinobenzyl)amino)-1-ethyl-1H-pyrazolo[4,3-d]pyrimidin-7(6H)-one

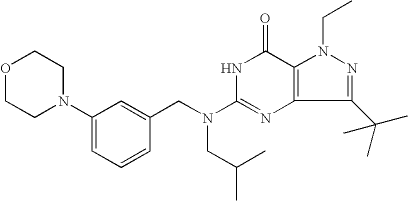

To 3-morpholin-4-yl-benzylamine (0.5 mmol) in THF (2 mL) at room temperature were added Iso-butylaldehyde (0.6 mmol) and anhydrous magnesium sulfate (60 mg). After stirring for 1.5 h at room temperature, sodium borohydride (0.5 mmol) was added and the mixture stirred for a further 2 h. Water (3 mL) was added to the mixture and stirring resumed for 10 min. Additional water was added (1 mL) and the mixture was extracted with CH₂Cl₂ (10 mL×3). After being dried over anhydrous magnesium sulfate the solvent was removed under reduced pressure to give the crude product. This material was used in subsequent steps without further purification.

The amine obtained in the previous step (0.26 mmol) was added to a solution of chloro-1-ethyl-3-tert-butyl-1,6-dihydro-pyrazolo[4,3-d]pyrimidin-7-one (0.13 mmol) in t-BuOH (0.5 mL). The reaction was heated in a sealed tube at 120° C. for 48 h. On completion of the reaction (monitored by LCMS), the mixture was allowed to cool to room temperature and the solvent was removed under reduced pressure. The final compound was isolated by preparative HPLC.

Preparative HPLC: Waters XBridge Prep C18 5 μm ODB 19 mm ID×100 mm L. The method used MeCN/H₂O 75-100% gradients. H₂O contained 0.1% trifluoroacetic acid (TFA).

### Example 125

### 5-((1H-1,2,4-triazol-1-yl)benzyl)(2-methylbutyl)amino)-3-cyclopropyl-1-methyl-1H-pyrazolo[4,3-d]pyrimidin-7(6H)-one

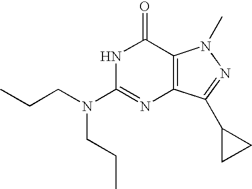

In a homogeneous solution of Pd(dppf)Cl₂ (0.012 mmol) and 3-bromo-5-dipropylamino-1-methyl-1,6-dihydro-pyrazolo[4,3-d]pyridine-7-one (0.122 mmol), dioxane (0.8 ml) and water (0.4 ml) was added potassium carbonate (0.244 mmol) and cyclopropaneboronic acid (0.244 mmol). The reaction was heated at 120° C. under microwave irradiation for 40 min. On cooling to room temperature, the solvents were removed under reduced pressure. The crude product was further purified by preparative HPLC. (yield 40%)

Preparative HPLC: Waters XBridge Prep C18 5 μm ODB 19 mm ID×100 mm L. The method used MeCN/H₂O 35-60% gradients. H₂O contained 0.1% trifluoroacetic acid (TFA).

In addition to the compounds exemplified above, various other pyrazolo[4,3-d]pyrimidinone compounds of this invention have been prepared using the procedure and synthetic methods described above, or via routine modification of the methods described here, and the corresponding starting materials, appropriate reagents, and purification methods known to those skilled in the art. The compounds prepared along with their analytical data and synthetic method information are listed in the Table 1, below.

TABLE 1
pyrazolo[4,3-d]pyrimidin-7(6H)-one derivatives

| | | | MS | | |
|---|---|---|---|---|---|
| Ex | | | (ES+) | NMR (in DMSO-d6 | Synthetic |
| # | STRUCTURE | MW | (obs) | unless specified) | Method |
| 1 | 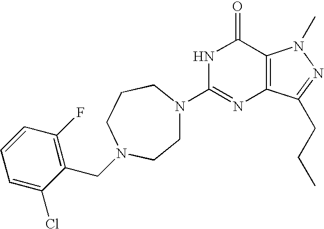 | 432.93 | 433.3(M + 1);HPLCrt =1.00 min. | 11.1 (1H, b, NH); 8.70(1H, m, Ar); 8.53 (1H,b, NH); 7.5-7.0 (3H, m,Ar); 4.1 (3H, s, CH₃);2.78 (2H, m, CH₂); 1.82(2H, m, CH₂); 1.01 (3H,t, J 7.3, CH₃). | A |
| 2 | 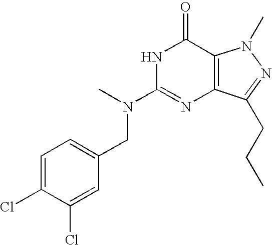 | 380.28 | 380(M + 1);rt =1.47 min. | NMR (¹H, CDCl₃) 11.2(1H, b, NH); 7.38 (1H,s, Ar); 7.32 (1H, d, J8.1; Ar); 7.10 (1H, m,Ar); 4.70 (2H, s, CH₂);3.99 (3H, s, CH₃); 3.07(3H, s, CH₃); 2.70 (2H,t, J 7.5, CH₂); 1.69 (2H,dt, J 7.3, 7.5, CH₂); 0.89(3H, t, J 7.3, CH₃). | A |
| 3 | 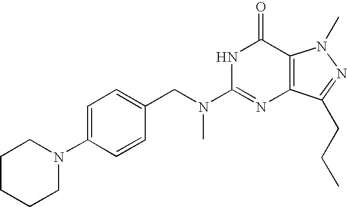 | 394.52 | 395.3(M + 1);HPLCrt =1.00 min. | NMR (¹H, CDCl₃) 9.0(1H, b, NH); 7.2 (2H, b,Ar); 6.85 (2H, b, Ar);4.6 (2H, s, CH₂); 4.0(3H, s, CH₃); 3.1 (4H,b, 2 × CH₂); 2.95 (3H, s,CH₃); 2.7 (2H, t, J 7.5,CH₂); 1.8-1.4 (8H, m,4 × CH₂); 0.9 (3H, t, J7.3, CH₃) | A |
| 4 | 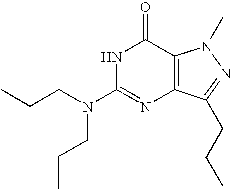 | 291.40 | 292.3(M + 1);HPLCrt =1.34 min. | NMR (¹H, CDCl₃) 3.9(3H, s, CH₃); 3.25 (4H,m, 2 × CH₂); 2.6 (2H, m,CH₂); 1. 6-1.4 (6H, m,3 = CH₂), 0.7 (9H, m,3 × CH₃). | A |
| 5 | 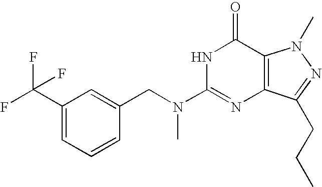 | 379.39 | 380(M + 1);HPLCrt =1.41 | NMR (¹H, CDCl₃) 8-7.4(5H, m, Ar); 4.9 (2H, s,CH₂); 4.1 (3H, s, CH₃);3.1 (3H, s, CH₃); 2.8(2H, m, CH₂), 1.8 (2H,m, CH₂); 0.97 (3H, t, J7.4, CH₃). | A |
| 6 | 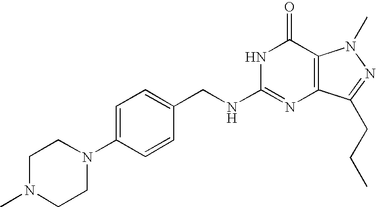 | 395.51 | 395(M + 1);HPLCrt =0.91 min | NMR (¹H, CDCl3) 7.23(2H, b, Ar); 6.70 (2H,b, Ar); 4.54 (2H, s,CH₂); 4.02 (3H, s, CH₃);3.50 (4H, b, 2 × CH₂);3.17 (2H, b, CH₂); 3.00(3H, s, CH₃); 2.71 (2H,t, J 7.7, CH₂); 1.6 (2H,m, CH₂); 0.89 (3H, t, J7, CH₃). | A |
| 7 | 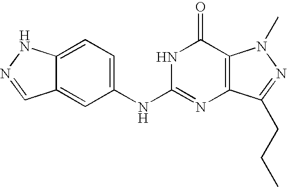 | 323.36 | 324.3(M + 1);HPLCrt =0.98 min. | 12.97 (1H, b, NH); 10.8(1H, b, NH); 8.46 (1H,b, NH); 8.26 (1H, s,Ar); 7.99 (1H, s, Ar);7.49 (1H, d, J 8.6, Ar);7.38 (1H, d, J 8.6, Ar);4.05 (3H, s, CH₃); 2.70(2H, m, CH₂); 1.76(2H, m, CH₂); 0.95(3H, t, J 7.3, CH₃) | A |
| 8 | 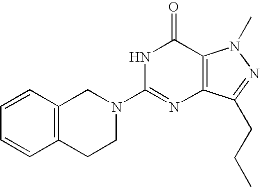 | 323.40 | 324.3(M + 1);HPLCrt =1.27 min | 11.2 (1H, b, NH); 7.18(4H, s, Ar); 4.67 (2H, s,CH₂); 4.03 (3H, s, CH₃);3.76 (2H, bt, CH₃); 2.90(2H, bt, CH₃); 2.65 (2H,m, CH₂); 1.71 (2H, m,CH₂); 0.91 (3H, m,CH₃). | A |
| 9 | 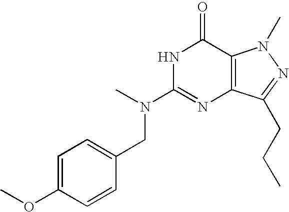 | 341.42 | 324.3(M + 1);HPLCrt =1.24 min. | 11.2 (1H, b, NH); 7.37(2H, d, J 8.2, Ar); 7.04(2H, d, J 8.2, Ar); 4.82(2H, s, CH₂); 4.18 (3H,s, CH₃); 3.88 (3H, s,CH₃); 3.12 (3H, s, CH₃);2.78 (2H, t, J 7.3, CH₂);1.84 (2H, m, CH₂); 1.05(3H, t, J 7.3, CH₃). | A |
| 10 | 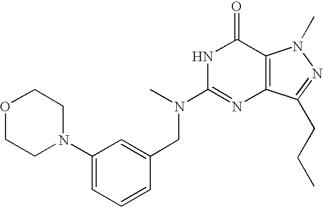 | 396.50 | 397.3(M + 1);HPLCrt =1.16 min | 10.9 (1H, b, NH); 2.17(1H, m, Ar); 7.85 (2H,m, Ar); 6.69 (1H, m,Ar); 4.68 (2H, s, CH2);4.02 (3H, s, CH₃); 3.71(4H, b, 2 × CH₂); 3.06(4H, b, 2 × CH₂); 2.98(3H, s, CH₃); 2.63 (2H,m, CH₂); 1.68 (2H, m,CH₂); 0.88 (3H, m,CH₃). | A |
| 11 | 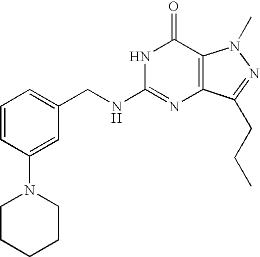 | 380.50 | 381.3(M + 1);HPLCrt =0.97 min | 7.3-6.9 (4H, m, Ar); 6.4(1H, b, NH); 4.27 (2H,s, CH₂); 3.79 (3H, s,CH₃); 3.12 (4H, b,CH₂); 2.40 (2H, m,CH₂); 1.6-1.3 (8H, m,4 × CH2); 0.67 (3H, m,CH₃). | A |
| 12 | 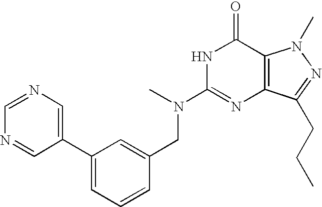 | 389.46 | 390.3M + 1;HPLCrt =1.13 min | 11.1 (1H, b, NH); 9.17(1H, s, Ar), 9.12 (2H, s,Ar); 7.78 (2H, d, J 7.3,Ar); 7.43 (2H, d, J 7.6,Ar); 4.82 (2H, s, CH₂);4.03 (3H, s, CH₃); 3.05(3H, s, CH₃); 2.63 (2H,m, CH₂); 1.67 (2H, m,CH₂), 0.89 (3H, t, J 6.4,CH₃). | A |
| 13 | 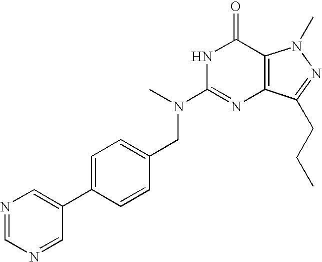 | 389.46 | 390.3(M + 1);HPLCrt =1.14 min. | 9.20 (1H, s, Ar); 9.18(2H, s, Ar); 7.8-7.7(2H, m, Ar); 7.6-7.4(2H, m, Ar); 4.82 (2H,s, CH₂); 4.02 (3H, s,CH₃); 3.07 (3H, s, CH₃);2.62 (2H, m, CH₂); 1.65(2H, m, CH₂); 0.83 (3H,m, CH₃). | A |
| 14 | 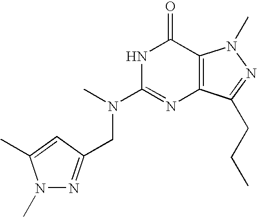 | 329.41 | 330.3(M + 1);HPLCrt =1.02 min | 5.87 (1H, s, Ar); 4.72(2H, s, CH₂); 4.02 (3H,s, CH₃); 3.71 (3H, s,CH₃); 3.01 (3H, s, CH₃);2.64 (2H, m, CH₂); 2.06(3H, s, CH₃); 1.68 (2H,m, CH₂); 0.90 (3H, t, J7.3, CH₃) | A |
| 15 | 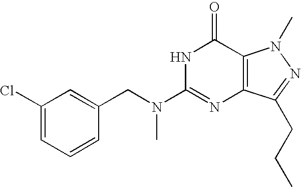 | 345.83 | 346(M + 1);HPLCrt =1.36 min. | 11.1 (1H, b, NH); 7.2-7.4 (5H, m, Ar); 4.7(2H, s, CH₂); 4.0 (3H, s,CH₃); 3.0 (3H, s, CH₃);2.6 (2H, t, J 7.3, CH₂);1.66 (2H, dt, J 7.3, 7.4,CH₂); 0.88 (3H, t, J 7.4,CH₃). | A |
| 16 | 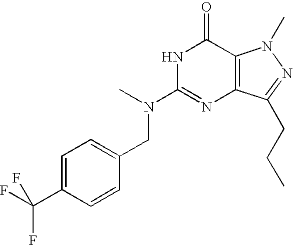 | 379.39 | 380.3(M + 1);HPLCrt =1.41 min | 11.1 (1H, b, NH); 7.69(2H, d, J 7.8, Ar); 7.47(2H, d, J 7.8, Ar); 4.8(2H, s, CH₂); 4.0 (3H, s,CH₃); 3.0 (3H, s, CH₃);2.6 (2H, t, J 7.3, CH₂);1.63 (2H, dt, J 7.3, 7.4,CH₂); 0.85 (3H, t, J 7.4,CH₃). | A |
| 17 | 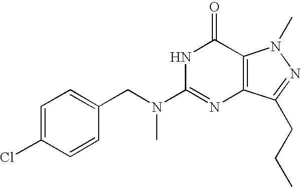 | 345.83 | 346.2(M + 1);HPLCrt =1.37 min | 10.9 (1H, b, NH); 7.38(2H, d, J 7.5, Ar); 7.29(2H, d, J 8, Ar); 4.72(2H, s, CH₂); 4.02 (3H,s, CH₃); 3.00 (3H, s,CH₃); 2.61 (2H, m,CH₂); 1.65 (2H, m,CH₂); 0.88 (3H, t, J 7.3,CH₃) | A |
| 18 | 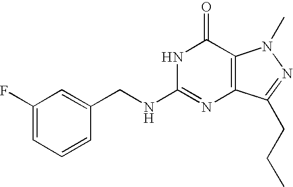 | 315.35 | 316.3(M + 1);HPLCrt =1.21 min | 7.4 (2H, m, Ar); 7.22(2H, m, Ar, NH); 7.1(1H, m, Ar); 6.6 (1H, b,NH); 4.47 (2H, d, J 5.5,CH₂); 3.99 (3H, s,CH₃); 2.60 (2H, m,CH₂); 1.62 (2H, m,CH₂); 0.86 (3H, t, J 7.3,CH₃). | A |
| 19 | 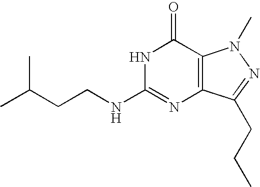 | 277.37 | 278.3(M + 1);HPLCrt = 1.14 min | 6.2 (1H, b, NH); 4.15(3H, s, CH₃); 3.45 (2H,m, CH₂); 2.78 (2H, m,CH₂); 1.9-1.7 (3H, m,CH₂, CH); 1.56 (2H, m,CH₂), 1.05 (9H, m,3 × CH₃). | A |
| 20 | 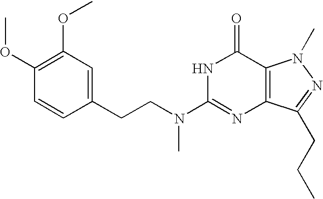 | 385.47 | 396.3(M + 1);HPLCrt =1.15 min | | A |
| 21 | 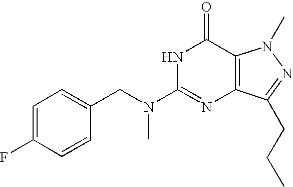 | 329.38 | 330.3(M + 1);HPLCrt =1.29 min. | | A |
| 22 | 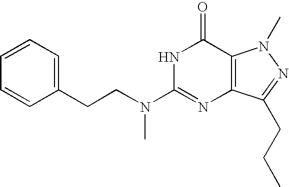 | 325.42 | 326.3(M + 1);HPLCrt =1.27 min. | 7.3-7.15 (5H, m, Ar);4.02 (3H, s, CH₃); 3.68(2H, m, CH₂); 3.02 (3H,s, CH₃); 2.86 (2H, m,CH₂); 2.66 (2H, m,CH₂); 1.72 (2H, m,CH₂); 0.92 (3H, m,CH₃) | A |
| 23 | 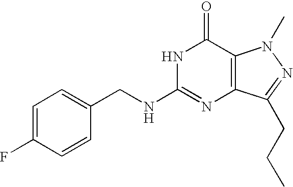 | 315.35 | 316.3(M + 1);HPLCrt =1.19 min. | 7.41 (2H, m, Ar); 7.14(2H, m, Ar); 6.55 (1H,b, NH); 4.45 (2H, d, J4.8, CH₂); 3.99 (3H, s,CH₃); 2.61 (2H, t, J 7.3,CH₂); 1.64 (2H, dt, J7.3, CH₂); 0.88 (3H, t, J7.3, CH₃). | A |
| 24 | 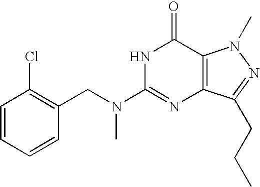 | 345.83 | 346.2(M + 1);HPLCrt =1.36 min | 7.46 (1H, m, Ar); 7.29(2H, m, Ar); 7.19 (1H,m, Ar); 4.81 (2H, s,CH₂); 4.01 (3H, s, CH₃);3.08 (3H, s, CH₃); 2.59(2H, t, J 7.2, CH₂); 1.62(2H, dt, J 7.2, 7.3, CH₂);0.85 (3H, t, J 7.3, CH₃). | A |
| 25 | 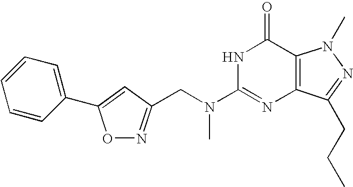 | 378.44 | 379.3(M + 1);HPLCrt =1.33 min. | 7.83 (2H, d, J 7.9, Ar);7.5 (3H, m, Ar); 6.93(1H, s, Ar); 4.82 (2H, s,CH₂); 4.03 (3H, s, CH₃);3.09 (3H, s, CH₃); 2.63(2H, d, J 7.3, CH₂); 1.66(2H, d, J 7.3, CH₂);0.85 (3H, t, J 7.3, CH₃). | A |
| 26 | 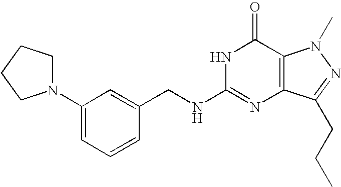 | 366.47 | 367.4(M + 1);HPLCrt =1.15 min. | | A |
| 27 | 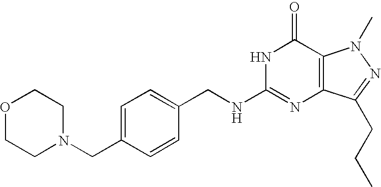 | 396.50 | 397.3(M + 1);HPLCrt =0.91 min. | 7.5 (4H, m, Ar); 4.78(2H, s, CH₂); 4.18 (2H, sCH₂); 4.11 (3H, s, CH₃);3.99 (4H, b, 2 × CH₂);3.43 (2H, b, CH₂); 2.85(2H, b, CH₂); 2.80 (2H,t, J 2.7, CH₂); 1.69 (2H,m, CH₂); 0.99 (3H, t, J7.3, CH₃). | A |
| 28 | 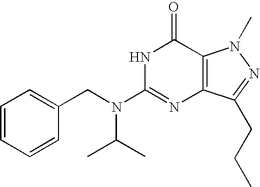 | 339.44 | 340.3(M + 1);HPLCrt =1.41 min. | | B |
| 29 | 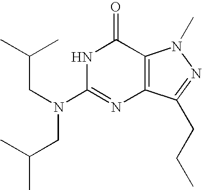 | 319.45 | 320.3(M + 1);HPLCrt =1.46 min. | | A |
| 30 | 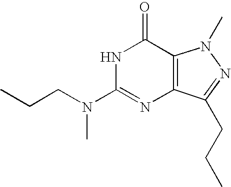 | 263.35 | 264.2(M + 1);HPLCrt =1.07 min. | | A |
| 31 | 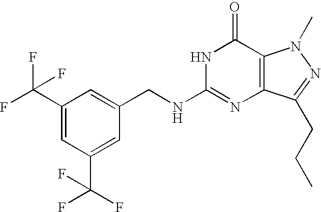 | 433.36 | 344.1(M + 1);HPLC:rt =1.46 min. | | A |
| 32 | 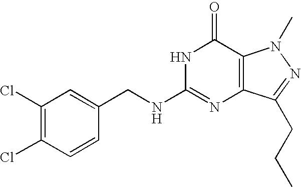 | 366.25 | 366.2(M);368.1(M + 2)HPLCrt =1.37 min. | | A |
| 33 | 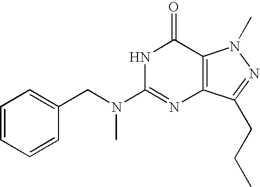 | 311.39 | 312.1(M + 1);HPLCrt =1.26 min. | | A |
| 34 | 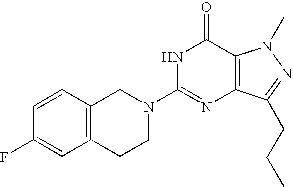 | 341.39 | 342.1(M + 1);HPLCrt =1.30 min. | | A |
| 35 | 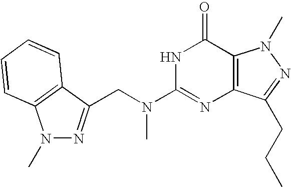 | 365.44 | 366.2(M + 1);HPLCrt =1.24 min. | | A |
| 36 | 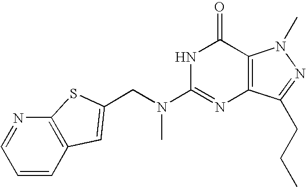 | 368.46 | 369.1(M + 1);HPLCrt =1.19 min. | | A |
| 37 | 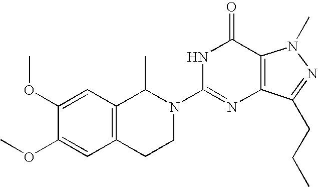 | 397.48 | 398.1(M + 1);HPLCrt =1.26 min. | 6.74 (1H, s, ArH); 6.71(1H, s, ArH); 5.40 (1H,m, CH); 4.21 (1H, m,CH2); 4.02 (3H, s,CH3); 3.74 (6H, underwater peak, 2 × CH3);3.32 (1H, m, CH); 2.85(1H, m, CH); 2.66 (3H,m, CH2 + CH), 1.71 (2H,m, CH2) 1.41 (3H, d, J6.4, CH3); 0.92 (3H, t, J7.6, CH3). | A |
| 38 | 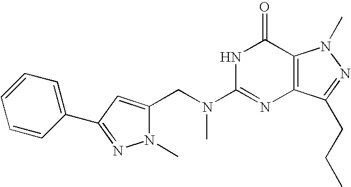 | 391.48 | 392.2(M + 1);HPLCrt =1.27 min. | | A |
| 39 | 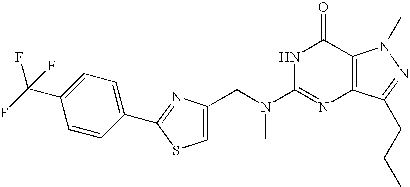 | 462.50 | 463.1(M + 1);HPLCrt =1.48 min. | | A |
| 40 | 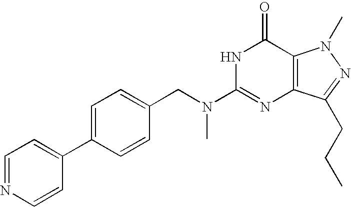 | 388.48 | 389.2(M + 1);HPLCrt =1.00 min. | | A |
| 41 | 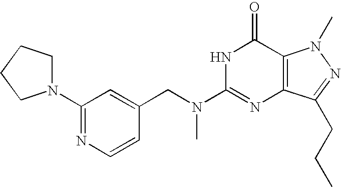 | 381.48 | 382.2(M + 1);HPLCrt =0.99 min. | | A |
| 42 | 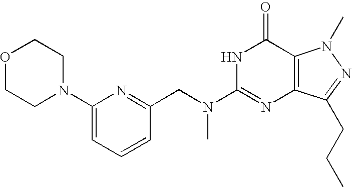 | 397.48 | 398.2(M + 1);HPLCrt =1.02 min. | | A |
| 43 | 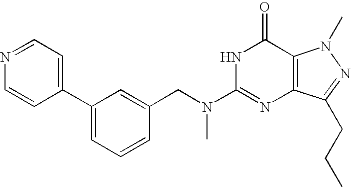 | 388.48 | 389.2(M + 1);HPLCrt =1.01 min. | | A |
| 44 | 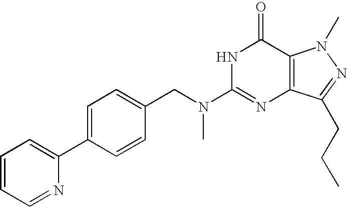 | 388.48 | 389.2(M + 1);rt =1.03 min. | | A |
| 45 | 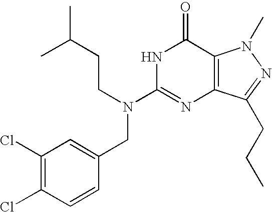 | 436.39 | 436.1(M + 1);HPLCrt =1.69 min. | 11.1 (1H, b, NH); 7.57(2H, m, ArH); 7.25 (1H,d, J 8.4, ArH); 4.68 (2H,s, CH2); 4.01 (3H, s,CH3); 3.51 (2H, m,CH2); 2.61 (2H, m,CH2); 1.61 (3H, m, CH,CH2); 1.39 (2H, m,CH2); 0.87 (9H, m,3 × CH3). | B |
| 46 | 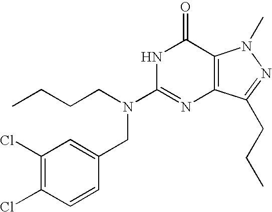 | 422.36 | 422.1(M + 1);HPLCrt =1.64 min. | | B |
| 47 | 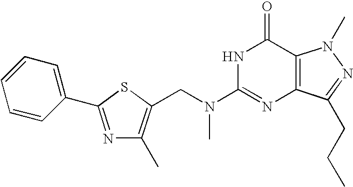 | 408.53 | 409.1(M + 1);HPLCrt =1.37 min. | 11.2 (1H, b, NH); 7.81(2H, m, ArH); 7.42 (3H,m, ArH); 4.84 (2H, s,CH2); 4.03 (3H, s,CH3); 3.04 (3H, s,CH3); 2.69 (2H, m,CH2); 2.5 (3H, underDMSO peak, CH3) 1.72(2H, m, CH2); 0.92 (3H,t, J 7.6, CH3). | A |
| 48 | 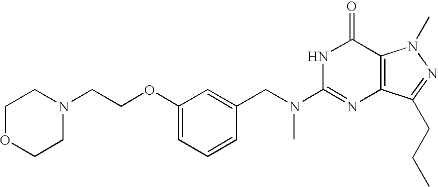 | 440.55 | 441.2(M + 1);HPLCrt =1.02 min. | | A |
| 49 | 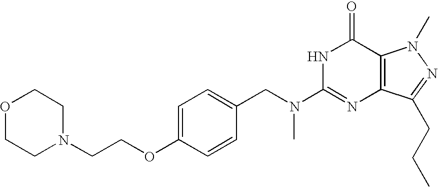 | 440.55 | 441.2(M + 1);HPLCrt =1.00 min. | | A |
| 50 | 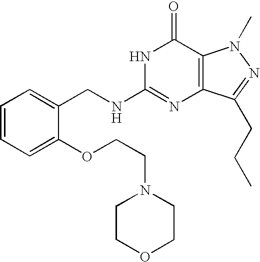 | 426.52 | 427.2(M + 1);HPLCrt =1.01 min. | | A |
| 51 | 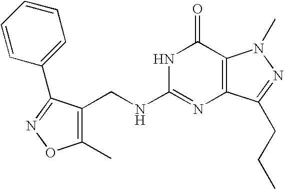 | 378.44 | 379.2(M + 1);HPLCrt =1.26 min. | | A |
| 52 | 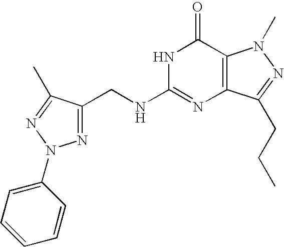 | 378.44 | 379.2(M + 1);HPLCrt =1.31 min. | 11.2 (1H, b, NH); 7.92(2H, d, J 8.4 ArH); 7.53(2H, t, J 7.6, ArH); 7.37(1H, m, ArH); 6.53 (1H,b, NH); 4.57 (2H, d, J5.2, CH2); 3.99 (3H, s,CH3); 2.69 (2H, m,CH2); 2.41 (3H, s,CH3); 1.65 (2H, m,CH2); 0.88 (3H, t, J7.6, CH3). | A |
| 53 | 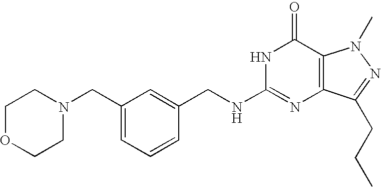 | 396.50 | 397.2(M + 1);HPLCrt =0.94 min. | | A |
| 54 | 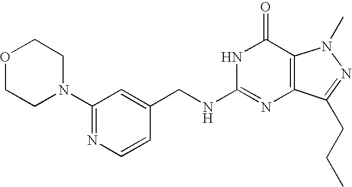 | 383.46 | 384.2(M + 1);HPLCrt =0.90 min. | | A |
| 55 | 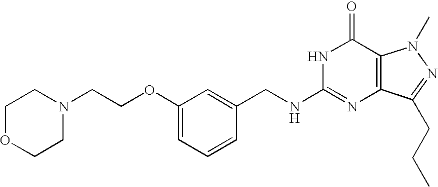 | 426.52 | 427.2(M + 1);HPLCrt =0.95 min. | | A |
| 56 | 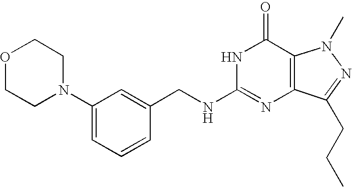 | 382.47 | 383.2(M + 1);HPLCrt =1.08 min. | | A |
| 57 | 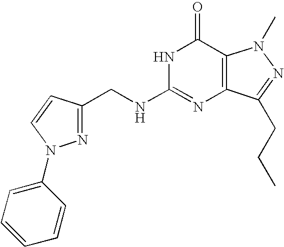 | 363.43 | 364.2(M + 1);HPLCrt =1.17 min. | | A |
| 58 | 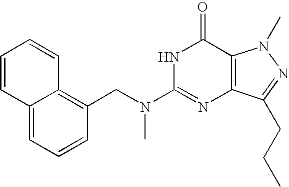 | 361.45 | 362.2(M + 1);HPLCrt =1.41 min. | | A |
| 59 | 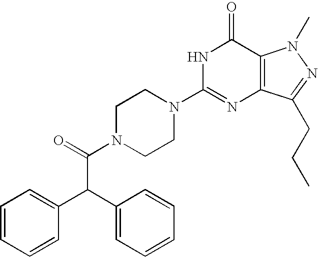 | 470.58 | 471.2(M + 1);HPLCrt =1.36 min. | 7.28 (10H, m, ArH);5.60 (1H, s, CH); 4.02(3H, s, CH3); 3.5 (4H,under peak of water,2 × CH2) 3.21 (2H, b,CH2); 2.90 (2H, b,CH2); 2.62 (2H, m,CH2) 1.67 (2H, m,CH2); 0.89 (3H, t, J7.6, CH3). | A |
| 60 | 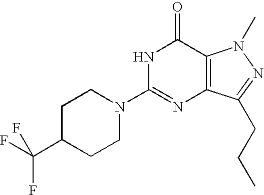 | 343.35 | 344.1(M + 1);HPLCrt =1.29 min. | | A |
| 61 | 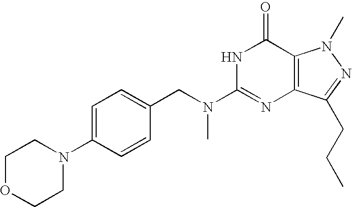 | 396.50 | 304.1(M + 1);HPLCrt =1.21 min. | | A |
| 62 | 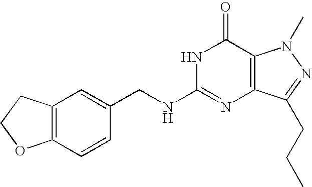 | 339.40 | 340.1(M + 1);HPLCrt =1.15 min. | | A |
| 63 | 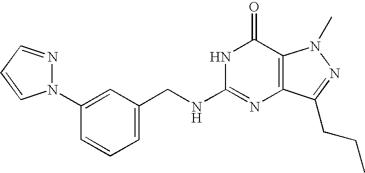 | 363.43 | 364.1(M + 1);HPLCrt =1.16 min. | | A |
| 64 | 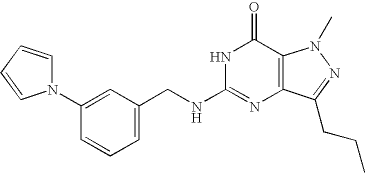 | 362.44 | 363.1(M + 1);HPLCrt =1.30 min. | | A |
| 65 | 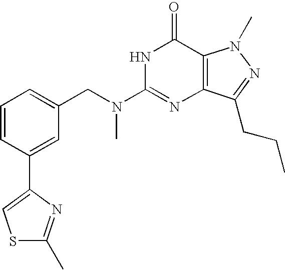 | 408.53 | 1.31(M + 1);HPLCrt = 409.1 | | A |
| 66 | 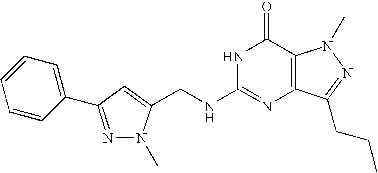 | 377.45 | 1.2(M + 1);HPLCrt = 378.1 | | A |
| 67 | 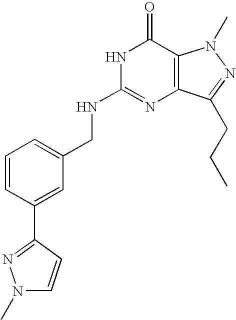 | 377.45 | 378.1(M + 1);HPLCrt = 1.13 | | A |
| 68 | 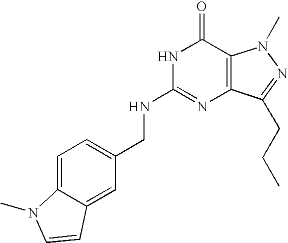 | 350.43 | 351.1(M + 1);HPLCrt = 1.18 | | A |
| 69 | 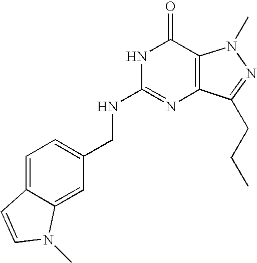 | 350.43 | 351.2(M + 1);HPLCrt = 1.2 | | A |
| 70 | 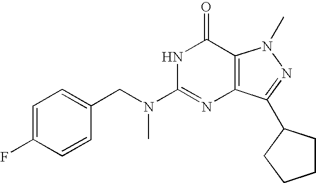 | 355.42 | 356(M + 1);HPLCrt = 3.82¹ | NMR (¹H, CDCl₃) 9.12(1H, s), 7.29 (2H, m),7.02 (2H, m), 4.74 (2H,s), 4.08 (3H, s), 3.28(1H, pent, J = 8 Hz),3.08 (3H, s), 2.06 (2H,m), 1.94 (2H, m), 1.83(2H, m), 1.64 (2H, m). | A |
| 71 | 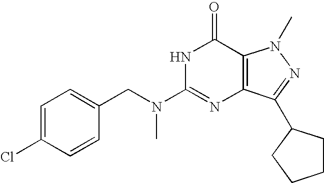 | 371.87 | 372.3(M + 1);HPLCrt = 4.06¹ | | A |
| 72 | 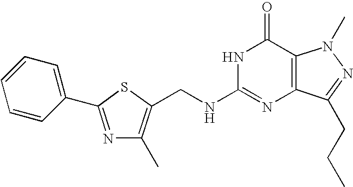 | 394.50 | 395.1(M + 1);HPLCrt = 1.29 | | A |
| 73 | 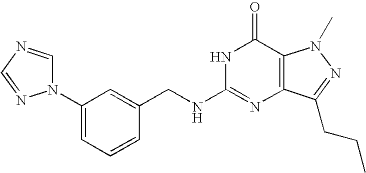 | 364.41 | 365.1(M + 1);HPLCrt = 1.24 | | A |
| 74 | 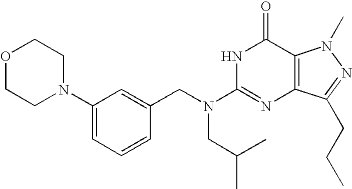 | 438.58 | 439.2(M + 1);HPLCrt = 1.41 | | B |
| 75 | 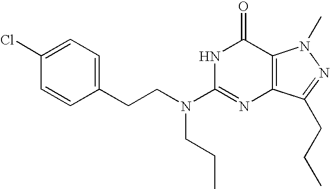 | 387.92 | 388.1(M + 1);HPLCrt = 1.55 | | B |
| 76 | 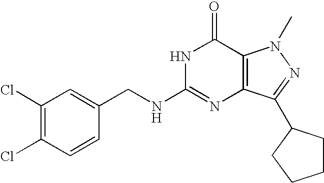 | 392.29 | 392(M + 1);HPLCrt = 4.01¹ | NMR (¹H, CDCl₃) 11.43(1H, s), 7.53 (1H, s),7.38 (1H, d, J = 8 Hz),7.23 (1H, dd, J = 8, 4Hz), 5.36 (1H, brs), 4.56(2H, d, J = 8 Hz), 4.08(3H, s), 3.25 (1H, pent,J = 8 Hz), 2.05 (2H, m),1.93-1.80 (4H, m), 1.66(2H, m). | A |
| 77 | 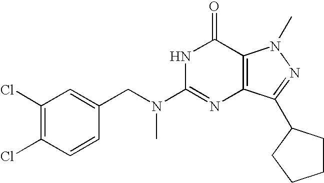 | 406.32 | :406(M + 1);HPLCrt = 4.31¹ | | A |
| 78 | 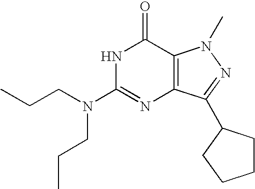 | 317.44 | 318(M + 1);HPLCrt = 4.20¹ | | A |
| 79 | 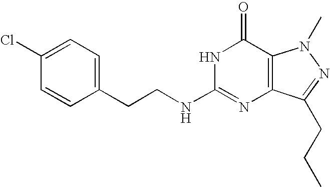 | 345.83 | 346.1(M + 1);HPLCrt = 1.28 | | A |
| 80 | 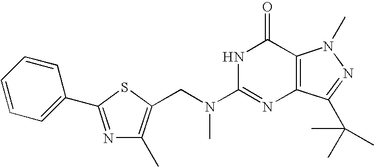 | 422.56 | 423.1(M + 1);HPLCrt = 1.51 | 11.16 (1H, b, NH); 7.79(2H, m, Ar); 7.43 (3H,m, Ar); 4.89 (2H, s,CH₂); 4.04 (3H, s, CH₃);3.02 (3H, s, CH₃); 2.47(3H, s, CH₃); 1.42 (9H,2, CH₃). | A |
| 81 | 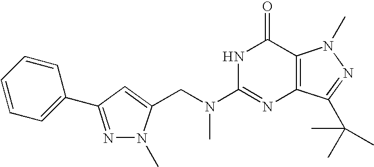 | 405.51 | 406.2(M + 1);HPLCrt = 1.4 | 7.71 (2H, d, J 7.2, Ar);7.34 (2H, dd, J 7.2, 7.6,Ar); 7.25 (1H, d, J 7.6,Ar); 6.56 (1H, s, Ar);4.82 (2H, s, CH₂); 4.01(3H, s, CH₃); 3.81 (3H,s, CH₃); 3.07 (3H, s,CH₃); 1.38 (9H, s, CH₃). | A |
| 82 | 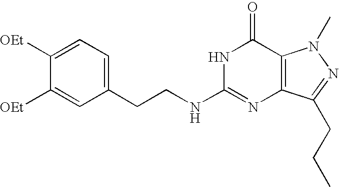 | 399.50 | 400.2(M + 1);HPLCrt = 1.23 | | A |
| 83 | 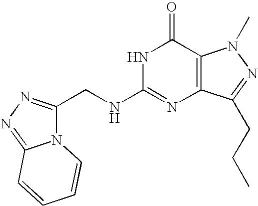 | 338.37 | 339.1(M + 1);HPLCrt = 0.9 | | A |
| 84 | 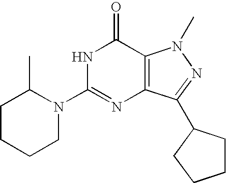 | 315.42 | 316(M + 1);HPLCrt = 3.95¹ | | A |
| 85 | 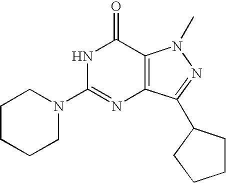 | 301.39 | 301(M + 1);HPLCrt = 367¹ | NMR (¹H, CDCl₃) 9.78(1H, s), 4.14 (3H, s),3.52 (4H, m), 3.29 (1H,pent, J = 8 Hz), 2.08(2H, m), 1.98-1.81 (4H,m), 1.67 (8H, m), 1.26(2H, m). | A |
| 86 | 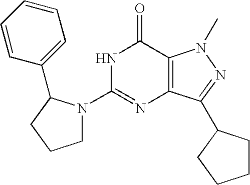 | 363.47 | 364(M + 1);HPLCrt = 3.87¹ | NMR (¹H, CDCl₃) 7.98(1H, s), 7.34 (2H, m),7.27 (3H, m), 4.92 (1H,dd, J = 8, 4 Hz), 4.08(3H, s), 3.88-3.77 (2H,m), 3.23 (1H, pent, J = 8Hz), 2.48 (1H, m), 2.09-1.96 (5H, m), 1.95-1.77(4H, m), 1.64 (2H, m) | A |
| 87 | 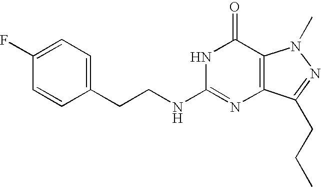 | 329.38 | 380.2(M + 1);HPLCrt =]1.31 | | A |
| 88 | 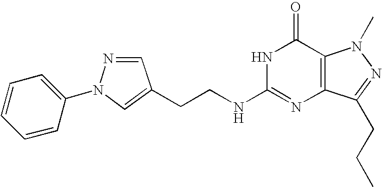 | 377.45 | 378.2(M + 1);HPLCrt = 1.16 | | A |
| 89 | 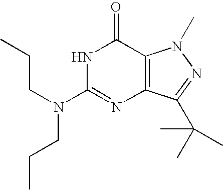 | 305.43 | 306.2(M + 1);HPLCrt = 1.56 | 3.99 (3H, s, CH₃); 3.39(4H, m, CH₂); 1.54 (4H,m, CH₂); 1.37 (9H, s,CH₃); 0.86 (6H, t, J 6.8,CH₃). | A |
| 90 | 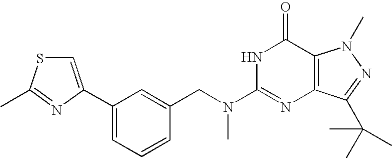 | 422.56 | 423.1(M + 1);HPLCrt = 1.5 | 11.08 (1H, b, NH); 7.90(1H, s, Ar); 7.86 (1H, s,Ar); 7.79 (1H, d, J 6.4,Ar); 7.37 (1H, dd, J 6.4,7.6, Ar); 7.23 (1H, d, J7.6 Ar); 4.79 (2H, s,CH₂); 4.01 (3H, s, CH₃);3.04 (3H, s, CH₃); 2.69(3H, s, CH₃); 1.36 (9H,s, (CH₃). | A |
| 91 | 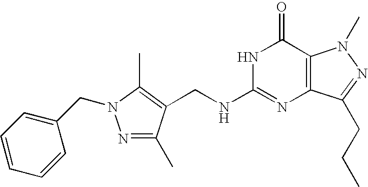 | 405.51 | 406.2(M + 1);HPLCrt = 1.17 | | A |
| 92 | 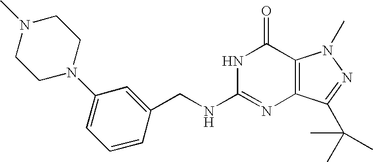 | 409.54 | 410.2(M + 1);HPLCrt = 1.06 | | A |
| 93 | 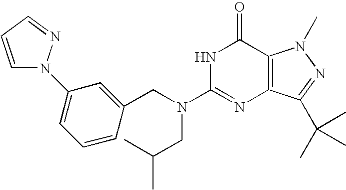 | 433.56 | 434.1(M + 1);HPLCrt = 1.58 | | B |
| 94 | 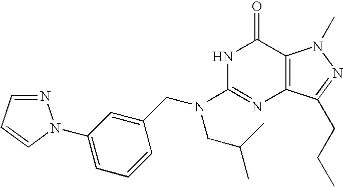 | 419.53 | 420(M + 1);HPLCrt = 1.46 | 11.03 (1H, b, NH); 8.45(1H, d, J 2.8, Ar); 7.78(1H, s, Ar); 7.72 (1H, d,J 1.6, Ar); 7.68 (1H, dd,J 2.2, 8.0, Ar); 7.42 (1H,t, J 7.6 Ar); 7.16 (1H, d,J 7.6, Ar); 6.53 (1H, dd,J 1.6, 2.8, Ar); 4.83 (2H,s, CH₂); 3.99 (3H, s,CH₃); 3.43 (2H, d, J 7.6,CH₂); 2.60 (2H, s, CH₂);2.03 (1H, m, CH); 1.63(2H, dt, J 7.6, 7.6,CH₂); 0.87 (6H, d, J6.8, CH₃); 0.84 (3H, t, J7.4, CH₃). | B |
| 95 | 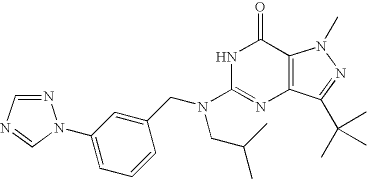 | 434.55 | 435.1(M + 1);HPLCrt = 1.44 | | B |
| 96 | 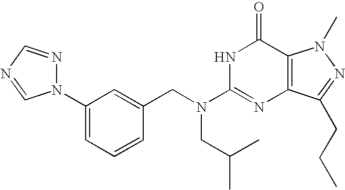 | 420.52 | 421(M + 1);HPLCrt = 1.32 | | B |
| 97 | 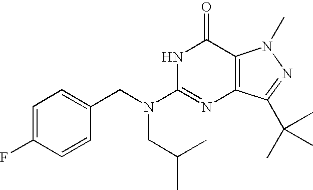 | 385.49 | 386(M + 1);HPLCrt = 1.65 | | B |
| 98 | 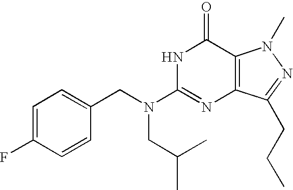 | 371.46 | 372(M + 1);HPLCrt = 1.51 | | B |
| 99 | 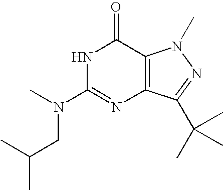 | 291.40 | 292.1(M + 1);HPLCrt = 1.47 | | B |
| 100 | 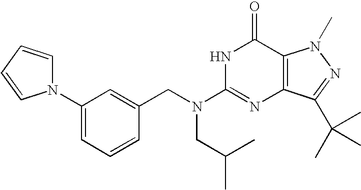 | 432.57 | 433(M + 1);HPLCrt = 1.68 | 11.03 (1H, b, NH); 7.43(1H, s, Ar); 7.40 (1H,m, Ar); 7.68 (1H, dd, J2.2, 8.0, Ar); 7.28 (2H,dd, J 2.4, 2.0, Ar); 7.09(1H, d, J 7.2, Ar); 6.53(1H, s, Ar); 6.25 (2H,dd, J 2.4, 2.0, Ar); 4.82(2H, s, CH₂); 3.99 (3H,s, CH₃); 3.41 (2H, d, J7.2, CH₂); 2.04 (1H, m,CH); 1.32 (9H,s, CH₃);0.88 (6H, d, J 6.8, CH₃). | B |
| 101 | 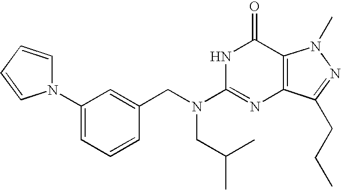 | 418.55 | 419(M + 1);HPLCrt = 1.57 | | B |
| 102 | 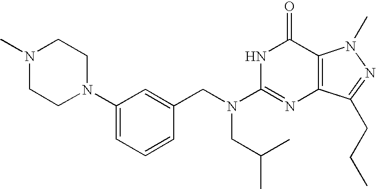 | 451.62 | 452.1(M + 1);HPLCrt = 1.15 | 7.19 (1H, t, J 8.0, Ar);6.91 (1H, s, Ar); 6.88(1H, d, J 7.6, Ar); 6.74(1H, d, J 8.0, Ar); 4.73(2H, s, CH₂); 4.03 (3H,s, CH₃); 3.76 (2H, m,CH₂); 3.51 (2H, m,CH₂); 3.32 (2H, d, J 7.2,CH₂); 3.14 (2H, m,CH₂); 2.90 (2H, m,CH₂); 2.85 (3H, d, J 3.6,CH₃); 2.62 (2H, t, J 7.2,CH₂); 2.03 (1H, m,CH); 1.67 (2H, dt, J 7.2,7.2, CH₂); 0.88 (3H, t, J7.2, CH₃); 0.86 (6H, d, J6.4, CH₃). | B |
| 103 | 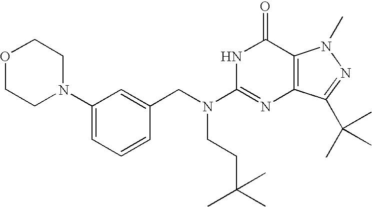 | 480.66 | 481.2(M + 1);HPLCrt = 1.67 | 10.98 (1H, s, NH); 7.18(1H, t, J 7.6, Ar); 6.83(2H, m, Ar); 6.68 (1H,d, J 8.0, Ar); 4.65 (2H,s, CH₂); 3.99 (3H, s,CH₃); 3.71 (4H, m,CH₂); 3.44 (2H, m,CH₂); 3.06 (4H, m,CH₂); 1.47 (2H, m,CH₂); 1.67 (9H, s, CH₃);0.87 (9H, s, CH₃). | B |
| 104 | 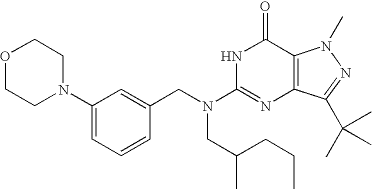 | 480.66 | 481.1(M + 1);HPLCrt = 1.68 | | B |
| 105 | 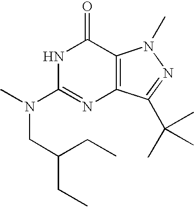 | 319.45 | 320(M + 1);HPLCrt = 1.61 | | B |
| 106 | 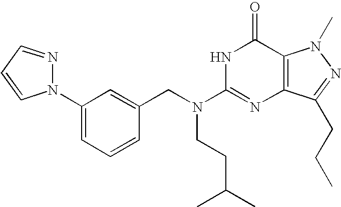 | 433.56 | 434.1(M + 1);HPLCrt = 1.54 | | B |
| 107 | 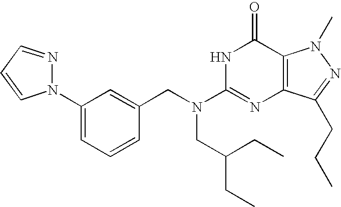 | 447.59 | 448.1(M + 1);HPLCrt = 1.59 | | B |
| 108 | 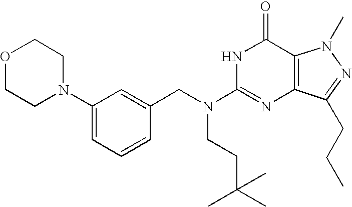 | 466.63 | 467.2(M + 1);HPLCrt = 1.56 | 10.97 (1H, s, NH); 7.18(1H, t, J 7.6, Ar); 6.85(2H, m, Ar); 6.67 (1H,d, J 7.6, Ar); 4.65 (2H,s, CH₂); 4.00 (3H, s,Ch₃); 3.72 (4H, m,CH₂); 3.43 (2H, m,CH₂); 3.06 (4H, m,CH₂); 2.63 (2H, m,CH₂); 1.45 (2H, m,CH₂); 0.88 (9H, s, CH₃). | B |
| 109 | 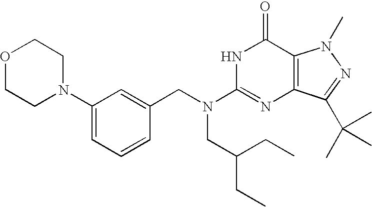 | 480.66 | 481.2(M + 1);HPLCrt = 1.69 | | B |
| 110 | 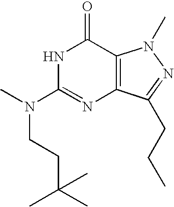 | 305.43 | 306(M + 1);HPLCrt = 1.37 | | B |
| 111 | 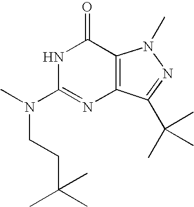 | 319.45 | 404(M + 1);HPLCrt = 1.66 | | B |
| 112 | 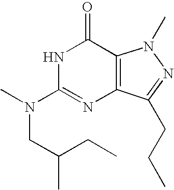 | 291.40 | 320(M + 1);HPLCrt = 1.62 | | B |
| 113 | 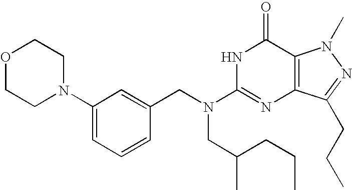 | 466.63 | 467.2(M + 1);HPLCrt = 1.56 | | B |
| 114 | 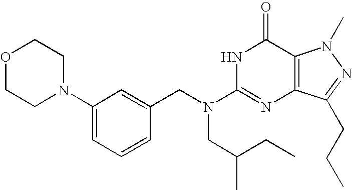 | 452.60 | 453.1(M + 1);HPLCrt = 1.49 | | B |
| 115 | 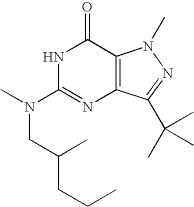 | 319.45 | 320.1(M + 1);HPLCrt = 1.63 | | B |
| 116 | 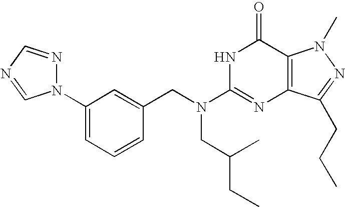 | 434.55 | 435.1(M + 1);HPLCrt = 1.39 | 9.26 (1H, s, Ar); 8.21(1H, s, Ar); 7.78 (1H, b,Ar); 7.72 (1H, d, J 8.0,Ar); 7.49 (1H, t, J 7.6Ar); 7.27 (1H, d, J 7.6,Ar); 4.83 (2H, s, CH₂);3.99 (3H, s, CH₃); 3.55(2H, m, CH₂); 2.59 (2H,t, J 7.6, CH₂); 1.82 (1H,m, CH); 1.61 (1H, dt, J7.6, 7.6, CH₂); 1.10 (1H,m, CH₂); 0.84 (3H, t, J6.4, CH₃); 0.81 (6H, t, J7.2, CH₃). | B |
| 117 | 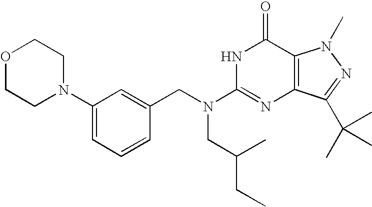 | 466.63 | 467.2(M + 1);HPLCrt = 1.62 | | B |
| 118 | 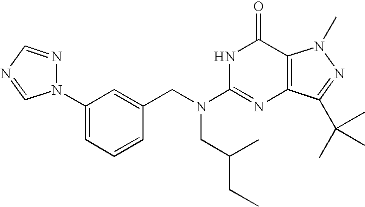 | 448.58 | 449.1(M + 1);HPLCrt = 1.52 | 11.07 (1H, s, NH); 9.26(1H, s, Ar); 8.21 (1H, s,Ar); 7.77 (1H, b, Ar);7.73 (1H, d, J 7.6, Ar);7.49 (1H, t, J 8.4 Ar);7.27 (1H, d, J 8.0, Ar);4.84 (2H, s, CH₂); 3.99(3H, s, CH₃); 3.55 (2H,m, CH₂); 1.85 (1H, m,CH); 1.40 (1H, m, CH₂);1.31 (9H, s, CH₃); 1.11(1H, m, CH₂); 0.84 (6H,t, J 6.0, CH₃). | B |
| 119 | 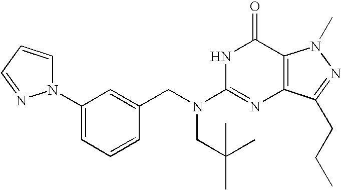 | 433.56 | 434.1(M + 1);HPLCrt = 1.52 | | B |
| 120 | 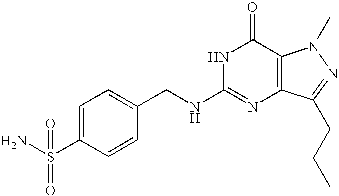 | 376.44 | 377(M + 1);HPLCrt = 0.97 | | A |
| 121 | 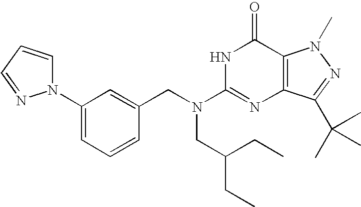 | 461.62 | 462.1(M + 1);HPLCrt = 1.71 | | B |
| 122 | 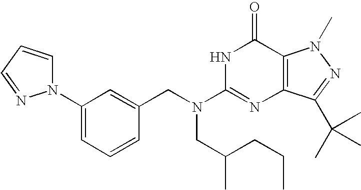 | 461.62 | 462.1(M + 1);HPLCrt = 1.72 | | B |
| 123 | 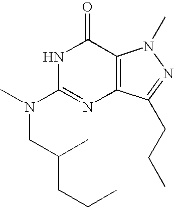 | 305.43 | 306(M + 1);HPLCrt = 1.4 | | B |
| 124 | 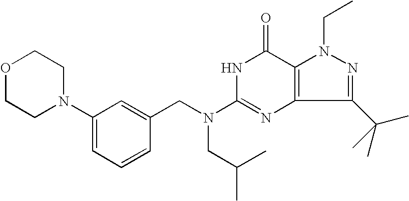 | 466.63 | 467.2(M + 1);HPLCrt = 1.63 | 10.93 (1H, s, NH); 7.16(1H, t, J 8.0, Ar); 6.83(2H, m, Ar); 6.65 (1H,d, J 7.6, Ar); 4.73 (2H,s, CH₂); 4.36 (2H, q, J7.2, CH₂); 3.71 (4H, m,2 × CH₂); 3.35 (2H, m,CH₂); 3.05 (4H, m,2 × CH₂); 2.01 (1H, m,CH); 1.36 (9H, s,3 × CH₃); 1.31 (3H, s,CH₃); 0.86 (6H, t, J 7.2,CH₃). | B |
| 125 | 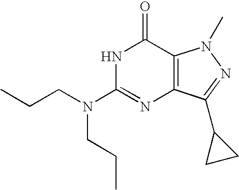 | 289.38 | 290.1(M + 1);HPLCrt = 1.3 | ) 3.96 (3H, s, CH₃);3.78 (4H, m, CH₂); 1.98(1H, m, CH); 1.52 (2H,m, CH₂); 1.05 (4H, s,CH₂); 0.86 (6H, t, J 7.2,CH₃). | C |

¹Xterra column, 100 mm × 4.6 mm × μ5 m 10 mM ammonium bicarbonate/acetonitrile gradient

### Biological Assays

### 2.1 In Vitro PDE1A Assay

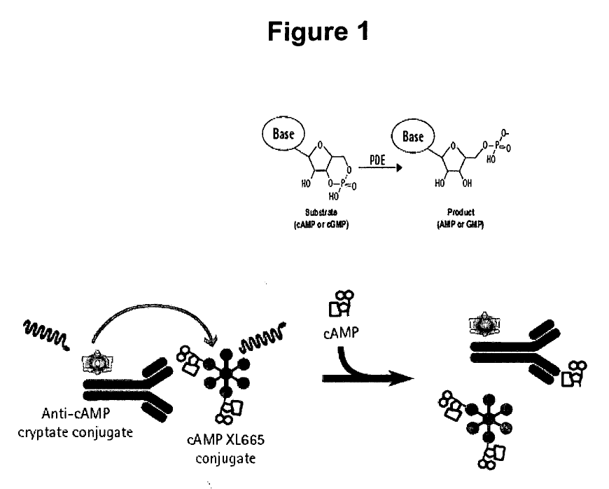

For the primary screening, an assay using the cAMP dynamic htrf kit from Cisbio (catnr 62AM2PEB) was used. Its principle is based on HTRF® technology (Homogeneous Time-Resolved Fluorescence). The method is a competitive immunoassay between native cAMP and the cAMP labeled with XL665. The tracer binding is visualized by a monoclonal antibody against cAMP, labeled with Cryptate. The specific signal (i.e. energy transfer) is inversely proportional to the concentration of cAMP in the sample (see FIG. 1).

For the enzymatic reaction a mix of 20 μL is made with purified PDE1A enzyme, 100 nM cAMP and the compound (10 μM in a final concentration of 1% DMSO) in a black 384-plate. The reaction buffer is Tris 20 mM pH 7.4, 4 μg/mL calmodulin, 3 mM MgCl₂, 1.5 mM CaCl₂, 0.2 mg/mL BSA and 0.001% Brij-35. After an incubation of 30 minutes at room temperature, the reaction is stopped by the addition of 10 μL labelled cAMP-XL665 and 10 μL anti-cAMP-Cryptate. After 1 hour incubation at room temperature, the readout is performed on the Envision (excitation 360 nm; emission donor 615 nm emission acceptor 665 nm).

PDE1A hydrolyses cAMP into 5′AMP; this low cAMP concentration will result in a high signal. A PDE1A inhibitor will result in a decrease of the signal.

As a positive control we used 10 μM Vardenafil (100% inhibition), as negative control is used 1% DMSO (0% inhibition), as variable control 10 μM Zaprinast (+/−50% inhibition) is used and as negative control compound 10 μM Ro-20-1724 (0% inhibition). The positive and negative control were used to calculate z′ and PIN values.

All compounds were screened in single at 10 μM. The hit criterium was set at PIN 50 (50% inhibition).

For the dose response and further screening, we used the Cyclic Nucleotide Phosphodiesterase Assay Kit from Biomol, a colorimetric, non-radioactive assay. The basis for the assay is the cleavage of cAMP by PDE1A. The 5′AMP is further cleaved into the nucleoside and phosphate by the enzyme 5′-nucleotidase (catnr KI-307). The phosphate released due to enzymatic cleavage is quantified using BIOMOL GREEN™ reagent (catnr AK-111) in a modified Malachite Green assay 1,2. A PDE1A inhibitor will result in a decrease of the signal.

For the enzymatic reaction a mix of 25 μL with purified PDE1A enzyme, 100 μM cAMP, 5′Nucleotidase and the compound is made in a clear 384-plate. The reaction buffer is Tris 20 mM pH 7.4, 4 μg/ml calmodulin, 3 mM MgCl2, 1.5 mM CaCl2, 0.2 mg/mL BSA and 0.001% Brij-35. After an incubation of 45 minutes at 37° C., the reaction is stopped by the addition of 50 μL BIOMOL GREEN™ reagent. After 30 minutes incubation at room temperature, the readout is performed on the Envision (absorption at 615 nm).

All compounds are tested in duplicate starting from 20 μM followed by a ⅓ serial dilution, 8 points (20 μM-6.67 μM-2.22 μM-740 nM-247 nM-82 nM-27 nM-9 nM) in a final concentration of 1% DMSO. As the positive dose response control compound Vardenafil is used. For the calculation of z′ and PIN values 10 μM Vardenafil is used as positive control (100% inhibition) and 1% DMSO as negative control (0% inhibition).

### Exemplary Compounds of the Invention

The following compounds have been or can be prepared according to the synthetic methods described above. For the purpose of Table 2 below, activity of each compound, which can be determined using the PDE1A assay method described in above, is expressed as follows:

“+”IC₅₀>500 nM

“++”IC₅₀101500 nM

“+++”IC₅₀<100 nM

TABLE 2
PDE1A IC₅₀ Data for Exemplary Compounds

| | | PDE1A |
|---|---|---|
| | | IC₅₀ |
| Ex # | NAME | (nM) |
| 1 | 5-(4-(2-chloro-6-fluorobenzyl)-1,4-diazepan-1-yl)-1- | + |
| | methyl-3-propyl-1H-pyrazolo[4,3-d]pyrimidin-7(6H)- | |
| | one | |
| 2 | 5-((3,4-dichlorobenzyl)(methyl)amino)-1-methyl-3- | +++ |
| | propyl-1H-pyrazolo[4,3-d]pyrimidin-7(6H)-one | |
| 3 | 1-methyl-5-(methyl(4-(piperidin-1-yl)benzyl)amino)-3- | ++ |
| | propyl-1H-pyrazolo[4,3-d]pyrimidin-7(6H)-one | |
| 4 | 5-(dipropylamino)-1-methyl-3-propyl-1H-pyrazolo[4,3- | +++ |
| | d]pyrimidin-7(6H)-one | |
| 5 | 1-methyl-5-(methyl(3-(trifluoromethyl)benzyl)amino)- | ++ |
| | 3-propyl-1H-pyrazolo[4,3-d]pyrimidin-7(6H)-one | |
| 6 | 1-methyl-5-(4-(4-methylpiperazin-1-yl)benzylamino)- | + |
| | 3-propyl-1H-pyrazolo[4,3-d]pyrimidin-7(6H)-one | |
| 7 | 5-(1H-indazol-5-ylamino)-1-methyl-3-propyl-1H- | + |
| | pyrazolo[4,3-d]pyrimidin-7(6H)-one | |
| 8 | 5-(3,4-dihydroisoquinolin-2(1H)-yl)-1-methyl-3- | ++ |
| | propyl-1H-pyrazolo[4,3-d]pyrimidin-7(6H)-one | |
| 9 | 5-((4-methoxybenzyl)(methyl)amino)-1-methyl-3- | +++ |
| | propyl-1H-pyrazolo[4,3-d]pyrimidin-7(6H)-one | |
| 10 | 1-methyl-5-(methyl(3-morpholinobenzyl)amino)-3- | ++ |
| | propyl-1H-pyrazolo[4,3-d]pyrimidin-7(6H)-one | |
| 11 | 1-methyl-5-(3-(piperidin-1-yl)benzylamino)-3-propyl- | ++ |
| | 1H-pyrazolo[4,3-d]pyrimidin-7(6H)-one | |
| 12 | 1-methyl-5-(methyl(3-(pyrimidin-5-yl)benzyl)amino)- | ++ |
| | 3-propyl-1H-pyrazolo[4,3-d]pyrimidin-7(6H)-one | |
| 13 | 1-methyl-5-(methyl(4-(pyrimidin-5-yl)benzyl)amino)- | ++ |
| | 3-propyl-1H-pyrazolo[4,3-d]pyrimidin-7(6H)-one | |
| 14 | 5-(((1,5-dimethyl-1H-pyrazol-3- | ++ |
| | yl)methyl)(methyl)amino)-1-methyl-3-propyl-1H- | |
| | pyrazolo[4,3-d]pyrimidin-7(6H)-one | |
| 15 | 5-((3-chlorobenzyl)(methyl)amino)-1-methyl-3-propyl- | ++ |
| | 1H-pyrazolo[4,3-d]pyrimidin-7(6H)-one | |
| 16 | 1-methyl-5-(methyl(4-(trifluoromethyl)benzyl)amino)- | ++ |
| | 3-propyl-1H-pyrazolo[4,3-d]pyrimidin-7(6H)-one | |
| 17 | 5-((4-chlorobenzyl)(methyl)amino)-1-methyl-3-propyl- | +++ |
| | 1H-pyrazolo[4,3-d]pyrimidin-7(6H)-one | |
| 18 | 5-(3-fluorobenzylamino)-1-methyl-3-propyl-1H- | + |
| | pyrazolo[4,3-d]pyrimidin-7(6H)-one | |
| 19 | 5-(isopentylamino)-1-methyl-3-propyl-1H- | + |
| | pyrazolo[4,3-d]pyrimidin-7(6H)-one | |
| 20 | 5-((3,4-dimethoxyphenethyl)(methyl)amino)-1-methyl- | + |
| | 3-propyl-1H-pyrazolo[4,3-d]pyrimidin-7(6H)-one | |
| 21 | 5-((4-fluorobenzyl)(methyl)amino)-1-methyl-3-propyl- | +++ |
| | 1H-pyrazolo[4,3-d]pyrimidin-7(6H)-one | |
| 22 | 1-methyl-5-(methyl(phenethyl)amino)-3-propyl-1H- | ++ |
| | pyrazolo[4,3-d]pyrimidin-7(6H)-one | |
| 23 | 5-(4-fluorobenzylamino)-1-methyl-3-propyl-1H- | + |
| | pyrazolo[4,3-d]pyrimidin-7(6H)-one | |
| 24 | 5-((2-chlorobenzyl)(methyl)amino)-1-methyl-3-propyl | ++ |
| | 1H-pyrazolo[4,3-d]pyrimidin-7(6H)-one | |
| 25 | 1-methyl-5-(methyl((5-phenylisoxazol-3- | +++ |
| | yl)methyl)amino)-3-propyl-1H-pyrazolo[4,3- | |
| | d]pyrimidin-7(6H)-one | |
| 26 | 1-methyl-3-propyl-5-(3-(pyrrolidin-1-yl)benzylamino)- | ++ |
| | 1H-pyrazolo[4,3-d]pyrimidin-7(6H)-one | |
| 27 | 1-methyl-5-(4-(morpholinomethyl)benzylamino)-3- | ++ |
| | propyl-1H-pyrazolo[4,3-d]pyrimidin-7(6H)-one | |
| 28 | 5-(benzyl(isopropyl)amino)-1-methyl-3-propyl-1H- | ++ |
| | pyrazolo[4,3-d]pyrimidin-7(6H)-one | |
| 29 | 5-(diisobutylamino)-1-methyl-3-propyl-1H- | ++ |
| | pyrazolo[4,3-d]pyrimidin-7(6H)-one | |
| 30 | 1-methyl-5-(methyl(propyl)amino)-3-propyl-1H- | ++ |
| | pyrazolo[4,3-d]pyrimidin-7(6H)-one | |
| 31 | 5-(3,5-bis(trifluoromethyl)benzylamino)-1-methyl-3- | + |
| | propyl-1H-pyrazolo[4,3-d]pyrimidin-7(6H)-one | |
| 32 | 5-(3,4-dichlorobenzylamino)-1-methyl-3-propyl-1H- | ++ |
| | pyrazolo[4,3-d]pyrimidin-7(6H)-one | |
| 33 | 5-(benzyl(methyl)amino)-1-methyl-3-propyl-1H- | ++ |
| | pyrazolo[4,3-d]pyrimidin-7(6H)-one | |
| 34 | 5-(6-fluoro-3,4-dihydroisoquinolin-2(1H)-yl)-1-methyl- | ++ |
| | 3-propyl-1H-pyrazolo[4,3-d]pyrimidin-7(6H)-one | |
| 35 | 1-methyl-5-(methyl((1-methyl-1H-indazol-3- | + |
| | yl)methyl)amino)-3-propyl-1H-pyrazolo[4,3- | |
| | d]pyrimidin-7(6H)-one | |
| 36 | 1-methyl-5-(methyl(thieno[2,3-b]pyridin-2- | ++ |
| | ylmethyl)amino)-3-propyl-1H-pyrazolo[4,3- | |
| | d]pyrimidin-7(6H)-one | |
| 37 | 5-(6,7-dimethoxy-1-methyl-3,4-dihydroisoquinolin- | ++ |
| | 2(1H)-yl)-1-methyl-3-propyl-1H-pyrazolo[4,3- | |
| | d]pyrimidin-7(6H)-one | |
| 38 | 1-methyl-5-(methyl((1-methyl-3-phenyl-1H-pyrazol-5- | +++ |
| | yl)methyl)amino)-3-propyl-1H-pyrazolo[4,3- | |
| | d]pyrimidin-7(6H)-one | |
| 39 | 1-methyl-5-(methyl((2-(4- | ++ |
| | (trifluoromethyl)phenyl)thiazol-4-yl)methyl)amino)-3- | |
| | propyl-1H-pyrazolo[4,3-d]pyrimidin-7(6H)-one | |
| 40 | 1-methyl-5-(methyl(4-(pyridin-4-yl)benzyl)amino)-3- | +++ |
| | propyl-1H-pyrazolo[4,3-d]pyrimidin-7(6H)-one | |
| 41 | 1-methyl-5-(methyl((2-(pyrrolidin-1-yl)pyridin-4- | + |
| | yl)methyl)amino)-3-propyl-1H-pyrazolo[4,3- | |
| | d]pyrimidin-7(6H)-one | |
| 42 | 1-methyl-5-(methyl((6-morpholinopyridin-2- | ++ |
| | yl)methyl)amino)-3-propyl-1H-pyrazolo[4,3- | |
| | d]pyrimidin-7(6H)-one | |
| 43 | 1-methyl-5-(methyl(3-(pyridin-4-yl)benzyl)amino)-3- | +++ |
| | propyl-1H-pyrazolo[4,3-d]pyrimidin-7(6H)-one | |
| 44 | 1-methyl-5-(methyl(4-(pyridin-2-yl)benzyl)amino)-3- | ++ |
| | propyl-1H-pyrazolo[4,3-d]pyrimidin-7(6H)-one | |
| 45 | 5-((3,4-dichlorobenzyl)(isopentyl)amino)-1-methyl-3- | +++ |
| | propyl-1H-pyrazolo[4,3-d]pyrimidin-7(6H)-one | |
| 46 | 5-(butyl(3,4-dichlorobenzyl)amino)-1-methyl-3-propyl- | ++ |
| | 1H-pyrazolo[4,3-d]pyrimidin-7(6H)-one | |
| 47 | 1-methyl-5-(methyl((4-methyl-2-phenylthiazol-5- | +++ |
| | yl)methyl)amino)-3-propyl-1H-pyrazolo[4,3- | |
| | d]pyrimidin-7(6H)-one | |
| 48 | 1-methyl-5-(methyl(3-(2- | + |
| | morpholinoethoxy)benzyl)amino)-3-propyl-1H- | |
| | pyrazolo[4,3-d]pyrimidin-7(6H)-one | |
| 49 | 1-methyl-5-(methyl(4-(2- | ++ |
| | morpholinoethoxy)benzyl)amino)-3-propyl-1H- | |
| | pyrazolo[4,3-d]pyrimidin-7(6H)-one | |
| 50 | 1-methyl-5-(2-(2-morpholinoethoxy)benzylamino)-3- | + |
| | propyl-1H-pyrazolo[4,3-d]pyrimidin-7(6H)-one | |
| 51 | 1-methyl-5-((5-methyl-3-phenylisoxazol-4- | ++ |
| | yl)methylamino)-3-propyl-1H-pyrazolo[4,3- | |
| | d]pyrimidin-7(6H)-one | |
| 52 | 1-methyl-5-((5-methyl-2-phenyl-2H-1,2,3-triazol-4- | +++ |
| | yl)methylamino)-3-propyl-1H-pyrazolo[4,3- | |
| | d]pyrimidin-7(6H)-one | |
| 53 | 1-methyl-5-(3-(morpholinomethyl)benzylamino)-3- | + |
| | propyl-1H-pyrazolo[4,3-d]pyrimidin-7(6H)-one | |
| 54 | 1-methyl-5-((2-morpholinopyridin-4-yl)methylamino)- | ++ |
| | 3-propyl-1H-pyrazolo[4,3-d]pyrimidin-7(6H)-one | |
| 55 | 1-methyl-5-(3-(2-morpholinoethoxy)benzylamino)-3- | ++ |
| | propyl-1H-pyrazolo[4,3-d]pyrimidin-7(6H)-one | |
| 56 | 1-methyl-5-(3-morpholinobenzylamino)-3-propyl-1H- | ++ |
| | pyrazolo[4,3-d]pyrimidin-7(6H)-one | |
| 57 | 1-methyl-5-((1-phenyl-1H-pyrazol-3-yl)methylamino)- | ++ |
| | 3-propyl-1H-pyrazolo[4,3-d]pyrimidin-7(6H)-one | |
| 58 | 1-methyl-5-(methyl(naphthalen-1-ylmethyl)amino)-3- | + |
| | propyl-1H-pyrazolo[4,3-d]pyrimidin-7(6H)-one | |
| 59 | 5-(4-(2,2-diphenylacetoyl)piperazin-1-yl)-1-methyl-3- | + |
| | propyl-1H-pyrazolo[4,3-d]pyrimidin-7(6H)-one | |
| 60 | 1-methyl-3-propyl-5-(4-(trifluoromethyl)piperidin-1- | + |
| | yl)-1H-pyrazolo[4,3-d]pyrimidin-7(6H)-one | |
| 61 | 1-methyl-5-(methyl(4-morpholinobenzyl)amino)-3- | ++ |
| | propyl-1H-pyrazolo[4,3-d]pyrimidin-7(6H)-one | |
| 62 | 5-((2,3-dihydrobenzofuran-5-yl)methylamino)-1- | ++ |
| | methyl-3-propyl-1H-pyrazolo[4,3-d]pyrimidin-7(6H)- | |
| | one | |
| 63 | 5-(1H-pyrazol-1-yl)benzylamino)-1-methyl-3-propyl- | ++ |
| | 1H-pyrazolo[4,3-d]pyrimidin-7(6H)-one | |
| 64 | 5-(1H-pyrrol-1-yl)benzylamino)-1-methyl-3-propyl- | ++ |
| | 1H-pyrazolo[4,3-d]pyrimidin-7(6H)-one | |
| 65 | 1-methyl-5-(methyl(3-(2-methylthiazol-4- | ++ |
| | yl)benzyl)amino)-3-propyl-1H-pyrazolo[4,3- | |
| | d]pyrimidin-7(6H)-one | |
| 66 | 1-methyl-5-((1-methyl-3-phenyl-1H-pyrazol-5- | +++ |
| | yl)methylamino)-3-propyl-1H-pyrazolo[4,3- | |
| | d]pyrimidin-7(6H)-one | |
| 67 | 1-methyl-5-(methyl-1H-pyrazol-3-yl)benzylamino)-3- | +++ |
| | propyl-1H-pyrazolo[4,3-d]pyrimidin-7(6H)-one | |
| 68 | 1-methyl-5-((1-methyl-1H-indol-5-yl)methylamino)-3- | ++ |
| | propyl-1H-pyrazolo[4,3-d]pyrimidin-7(6H)-one | |
| 69 | 1-methyl-5-((1-methyl-1H-indol-6-yl)methylamino)-3- | ++ |
| | propyl-1H-pyrazolo[4,3-d]pyrimidin-7(6H)-one | |
| 70 | 3-cyclopentyl-5-((4-fluorobenzyl)(methyl)amino)-1- | +++ |
| | methyl-1H-pyrazolo[4,3-d]pyrimidin-7(6H)-one | |
| 71 | 5-((4-chlorobenzyl)(methyl)amino)-3-cyclopentyl-1- | +++ |
| | methyl-1H-pyrazolo[4,3-d]pyrimidin-7(6H)-one | |
| 72 | 1-methyl-5-((4-methyl-2-phenylthiazol-5- | +++ |
| | yl)methylamino)-3-propyl-1H-pyrazolo[4,3- | |
| | d]pyrimidin-7(6H)-one | |
| 73 | 5-(1H-1,2,4-triazol-1-yl)benzylamino)-1-methyl-3- | ++ |
| | propyl-1H-pyrazolo[4,3-d]pyrimidin-7(6H)-one | |
| 74 | 5-(isobutyl(3-morpholinobenzyl)amino)-1-methyl-3- | +++ |
| | propyl-1H-pyrazolo[4,3-d]pyrimidin-7(6H)-one | |
| 75 | 5-((4-chlorophenethyl)(propyl)amino)-1-methyl-3- | ++ |
| | propyl-1H-pyrazolo[4,3-d]pyrimidin-7(6H)-one | |
| 76 | 3-cyclopentyl-5-(3,4-dichlorobenzylamino)-1-methyl- | ++ |
| | 1H-pyrazolo[4,3-d]pyrimidin-7(6H)-one | |
| 77 | 3-cyclopentyl-5-((3,4-dichlorobenzyl)(methyl)amino)- | +++ |
| | 1-methyl-1H-pyrazolo[4,3-d]pyrimidin-7(6H)-one | |
| 78 | 3-cyclopentyl-5-(dipropylamino)-1-methyl-1H- | +++ |
| | pyrazolo[4,3-d]pyrimidin-7(6H)-one | |
| 79 | 5-(4-chlorophenethylamino)-1-methyl-3-propyl-1H- | ++ |
| | pyrazolo[4,3-d]pyrimidin-7(6H)-one | |
| 80 | 3-tert-butyl-1-methyl-5-(methyl((4-methyl-2- | +++ |
| | phenylthiazol-5-yl)methyl)amino)-1H-pyrazolo[4,3- | |
| | d]pyrimidin-7(6H)-one | |
| 81 | 3-tert-butyl-1-methyl-5-(methyl((1-methyl-3-phenyl- | +++ |
| | 1H-pyrazol-5-yl)methyl)amino)-1H-pyrazolo(4,3- | |
| | d]pyrimidin-7(6H)-one | |
| 82 | 5-(3,4-diethoxyphenethylamino)-1-methyl-3-propyl | + |
| | 1H-pyrazolo[4,3-d]pyrimidin-7(6H)-one | |
| 83 | 5-([1,2,4]triazolo[4,3-a]pyridin-3-ylmethylamino)-1- | ++ |
| | methyl-3-propyl-1H-pyrazolo[4,3-d]pyrimidin-7(6H)- | |
| | one | |
| 84 | 3-cyclopentyl-1-methyl-5-(2-methylpiperidin-1-yl)-1H- | + |
| | pyrazolo[4,3-d]pyrimidin-7(6H)-one | |
| 85 | 3-cyclopentyl-1-methyl-5-(piperidin-1-yl)-1H- | + |
| | pyrazolo[4,3-d]pyrimidin-7(6H)-one | |
| 86 | 3-cyclopentyl-1-methyl-5-(2-phenylpyrrolidin-1-yl)- | +++ |
| | 1H-pyrazolo[4,3-d]pyrimidin-7(6H)-one | |
| 87 | 5-(4-fluorophenethylamino)-1-methyl-3-propyl-1H- | + |
| | pyrazolo[4,3-d]pyrimidin-7(6H)-one | |
| 88 | 1-methyl-5-(2-(1-phenyl-1H-pyrazol-4-yl)ethylamino)- | + |
| | 3-propyl-1H-pyrazolo[4,3-d]pyrimidin-7(6H)-one | |
| 89 | 3-tert-butyl-5-(dipropylamino)-1-methyl-1H- | +++ |
| | pyrazolo[4,3-d]pyrimidin-7(6H)-one | |
| 90 | 3-tert-butyl-1-methyl-5-(methyl(3-(2-methylthiazol-4- | +++ |
| | yl)benzyl)amino)-1H-pyrazolo[4,3-d]pyrimidin-7(6H)- | |
| | one | |
| 91 | 5-((1-benzyl-3,5-dimethyl-1H-pyrazol-4- | ++ |
| | yl)methylamino)-1-methyl-3-propyl-1H-pyrazolo[4,3- | |
| | d]pyrimidin-7(6H)-one | |
| 92 | 3-tert-butyl-1-methyl-5-(3-(4-methylpiperazin-1- | +++ |
| | yl)benzylamino)-1H-pyrazolo[4,3-d]pyrimidin-7(6H)- | |
| | one | |
| 93 | 5-((1H-pyrazol-1-yl)benzyl)(isobutyl)amino)-3-tert- | +++ |
| | butyl-1-methyl-1H-pyrazolo[4,3-d]pyrimidin-7(6H)- | |
| | one | |
| 94 | 5-((1H-pyrazol-1-yl)benzyl)(isobutyl)amino)-1-methyl- | +++ |
| | 3-propyl-1H-pyrazolo[4,3-d]pyrimidin-7(6H)-one | |
| 95 | 5-((1H-1,2,4-triazol-1-yl)benzyl)(isobutyl)amino)-3- | +++ |
| | tert-butyl-1-methyl-1H-pyrazolo[4,3-d]pyrimidin- | |
| | 7(6H)-one | |
| 96 | 5-((1H-1,2,4-triazol-1-yl)benzyl)(isobutyl)amino)-1- | +++ |
| | methyl-3-propyl-1H-pyrazolo[4,3-d]pyrimidin-7(6H)- | |
| | one | |
| 97 | 3-tert-butyl-5-((4-fluorobenzyl)(isobutyl)amino)-1- | +++ |
| | methyl-1H-pyrazolo[4,3-d]pyrimidin-7(6H)-one | |
| 98 | 5-((4-fluorobenzyl)(isobutyl)amino)-1-methyl-3- | +++ |
| | propyl-1H-pyrazolo[4,3-d]pyrimidin-7(6H)-one | |
| 99 | 3-tert-butyl-5-(isobutyl(methyl)amino)-1-methyl-1H- | +++ |
| | pyrazolo[4,3-d]pyrimidin-7(6H)-one | |
| 100 | 5-((1H-pyrrol-1-yl)benzyl)(isobutyl)amino)-3-tert- | +++ |
| | butyl-1-methyl-1H-pyrazolo[4,3-d]pyrimidin-7(6H)- | |
| | one | |
| 101 | 5-((1H-pyrrol-1-yl)benzyl)(isobutyl)amino)-1-methyl- | +++ |
| | 3-propyl-1H-pyrazolo[4,3-d]pyrimidin-7(6H)-one | |
| 102 | 5-(isobutyl(3-(4-methylpiperazin-1-yl)benzyl)amino)- | +++ |
| | 1-methyl-3-propyl-1H-pyrazolo[4,3-d]pyrimidin- | |
| | 7(6H)-one | |
| 103 | 3-tert-butyl-5-((3,3-dimethylbutyl)(3- | +++ |
| | morpholinobenzyl)amino)-1-methyl-1H-pyrazolo[4,3- | |
| | d]pyrimidin-7(6H)-one | |
| 104 | 3-tert-butyl-1-methyl-5-((2-methylpentyl)(3- | +++ |
| | morpholinobenzyl)amino)-1H-pyrazolo[4,3- | |
| | d]pyrimidin-7(6H)-one | |
| 105 | 3-tert-butyl-5-((2-ethylbutyl)(methyl)amino)-1-methyl- | +++ |
| | 1H-pyrazolo[4,3-d]pyrimidin-7(6H)-one | |
| 106 | 5-((1H-pyrazol-1-yl)benzyl)(isopentyl)amino)-1- | +++ |
| | methyl-3-propyl-1H-pyrazolo[4,3-d]pyrimidin-7(6H)- | |
| | one | |
| 107 | 5-((1H-pyrazol-1-yl)benzyl)(2-ethylbutyl)amino)-1- | +++ |
| | methyl-3-propyl-1H-pyrazolo[4,3-d]pyrimidin-7(6H)- | |
| | one | |
| 108 | 5-((3,3-dimethylbutyl)(3-morpholinobenzyl)amino)-1- | +++ |
| | methyl-3-propyl-1H-pyrazolo[4,3-d]pyrimidin-7(6H)- | |
| | one | |
| 109 | 3-tert-butyl-5-((2-ethylbutyl)(3- | +++ |
| | morpholinobenzyl)amino)-1-methyl-1H-pyrazolo[4,3- | |
| | d]pyrimidin-7(6H)-one | |
| 110 | 5-((3,3-dimethylbutyl)(methyl)amino)-1-methyl-3- | +++ |
| | propyl-1H-pyrazolo[4,3-d]pyrimidin-7(6H)-one | |
| 111 | 3-tert-butyl-5-((3,3-dimethylbutyl)(methyl)amino)-1- | +++ |
| | methyl-1H-pyrazolo[4,3-d]pyrimidin-7(6H)-one | |
| 112 | 1-methyl-5-(methyl(2-methylbutyl)amino)-3-propyl- | ++ |
| | 1H-pyrazolo[4,3-d]pyrimidin-7(6H)-one | |
| 113 | 1-methyl-5-((2-methylpentyl)(3- | +++ |
| | morpholinobenzyl)amino)-3-propyl-1H-pyrazolo[4,3- | +++ |
| | d]pyrimidin-7(6H)-one | |
| 114 | 1-methyl-5-((2-methylbutyl)(3- | +++ |
| | morpholinobenzyl)amino)-3-propyl-1H-pyrazolo[4,3- | |
| | d]pyrimidin-7(6H)-one | |
| 115 | 3-tert-butyl-1-methyl-5-(methyl(2- | +++ |
| | methylpentyl)amino)-1H-pyrazolo[4,3-d]pyrimidin- | |
| | 7(6H)-one | |
| 116 | 5-((1H-1,2,4-triazoi-1-yl)benzyl)(2- | +++ |
| | methylbutyl)amino)-1-methyl-3-propyl-1H- | |
| | pyrazolo[4,3-d]pyrimidin-7(6H)-one | |
| 117 | 3-tert-butyl-1-methyl-5-((2-methylbutyl)(3- | +++ |
| | morpholinobenzyl)amino)-1H-pyrazolo[4,3- | |
| | d]pyrimidin-7(6H)-one | |
| 118 | 5-((1H-1,2,4-triazol-1-yl)benzyl)(2- | +++ |
| | methylbutyl)amino)-3-tert-butyl-1-methyl-1H- | |
| | pyrazolo[4,3-d]pyrimidin-7(6H)-one | |
| 119 | 5-((1H-pyrazol-1-yl)benzyl)(neopentyl)amino)-1- | +++ |
| | methyl-3-propyl-1H-pyrazolo[4,3-d]pyrimidin-7(6H)- | |
| | one | |
| 120 | 4-((1-methyl-7-oxo-3-propyl-6,7-dihydro-1H- | + |
| | pyrazolo[4,3-d]pyrimidin-5- | |
| | ylamino)methyl)benzenesulfonamide | |
| 121 | 5-((1H-pyrazol-1-yl)benzyl)(2-ethylbutyl)amino)-3- | +++ |
| | tert-butyl-1-methyl-1H-pyrazolo[4,3-d]pyrimidin- | |
| | 7(6H)-one | |
| 122 | 5-((1H-pyrazol-1-yl)benzyl)(2-methylpentyl)amino)-3- | +++ |
| | tert-butyl-1-methyl-1H-pyrazolo[4,3-d]pyrimidin- | |
| | 7(6H)-one | |
| 123 | 1-methyl-5-(methyl(2-methylpentyl)amino)-3-propyl- | ++ |
| | 1H-pyrazolo[4,3-d]pyrimidin-7(6H)-one | |
| 124 | 3-tert-butyl-1-ethyl-5-(isobutyl(3- | + |
| | morpholinobenzyl)amino)-1H-pyrazolo[4,3- | |
| | d]pyrimidin-7(6H)-one | |
| 125 | 3-cyclopropyl-5-(dipropylamino)-1-methyl-1H- | ++ |
| | pyrazolo[4,3-d]pyrimidin-7(6H)-one | |

### 2.2 PDE1A Cellular Assay

As an alternative assay to the in vitro assay described above, a cellular PDE1A assay was developed in order to determine PDE1A inhibitor activity on cAMP levels in forskolin (NIHK477) stimulated HEK293 cells transiently transfected with PDE1A with HTRF® (Homogeneous Time-Resolved Fluorescence) cAMP dynamic 2 bulk kit (from Cisbio).

HEK 293T are routinely maintained in Dulbecco's Modified Eagle's Medium (DMEM) containing 10% heat inactivated fetal calf serum, 100 U/mL Penicillin and 100 μg/ml Streptomycin.

70% confluent HEK 293T are used for reverse transfection that involves simultaneously transfecting and plating cells. 60 000 cells are transiently transfected with 100 ng of pcDNA3.1 (+)PDE1 A DNA using 0.2 μL Jet-PEI (Polyplus) as transfection reagent per well for 96 well plate format. Transfected cells were seeded in poly-D-lysine coated 96-well plates. After overnight incubation at 37° C., 10% CO₂, transfection medium was removed and 100 μl of cell culture medium were added.

8 hour after transfection, the medium is removed, 20 μL DMEM is added, then 20 μL of compound dilution in DMEM, 50 mM Hepes (25 mM f.c.), 2*10⁻4M Forskolin (10⁻⁴M f.c.; NHK477; to activate adenylate cyclase) and 2*10⁻³M Rolipram (10⁻³M f.c.). By pharmacologically inhibiting PDE4 endogenous activity with Rolipram, transiently expressed PDE1A becomes the main PDE using cAMP as substrate in transfected HEK293T cells and this allows a specific PDE1A readout.

All compounds are tested in duplicate starting from 100 μM followed by a ⅓ serial dilution, 8 points (100 μM-33.3 μM-11.1 μM-3.7 μM-1.2 μM-411 nM-137 nM-46 nM-15 nM) in a final concentration of 5% DMSO.

After 1 h incubation at 37° C., cells are lysed by addition of 40 μL lysis buffer (50 mM KH2PO4, 50 mM Na₂HPO₄, 0.8 M Potassium Fluoride, 0.2% BSA, 1% Triton-X100). Cell lysates are diluted 1/100 (2× 1/10) in lysis buffer.

Htrf reagents are added to 20 L diluted cell lysate: 10 μL cAMP-d2 (cAMP labelled with d2 dye), then 10 μL anti cAMP-Cryptate (monoclonal antibody against cAMP, labeled with cryptate). The plates are incubated at room temperature for 1 hour and read on the Envision ((excitation 360 nm; emission donor 615 nm; emission acceptor 665 nm). Results are calculated from the 665 nm/615 nm ratio and expressed in Delta F % as described by manufacturer.

Appropriate positive and negative control may be selected by a person of skill in the art and used to calculate z′ and PIN values.

### 2.3 PDE Selectivity Panel

In one aspect the compounds of the invention are more potent against PDE1A than against other PDE isoforms. In a particular embodiment, the compounds are 2 fold more potent against PDE1A than against one or more of the other isoforms. In an alternative embodiment, the compounds of the invention are 5 fold, particularly 10-fold, particularly 20-fold more potent against PDE1A than against one or more of the other isoforms of PDE. In particular, the compounds of the invention are more potent against PDE1A than against at least one of PDE1B, PDE2A, PDE4A or PDE5A. In particular the compounds are more potent against PDE1A than against at least two of PDE1B, PDE2A, PDE4A or PDE5A. In particular the compounds are more potent against PDE1A than against all of the other PDE isoforms. Methods for testing the selectivity of the compounds against a range of PDE isoforms will be familiar to those of skill in the art, and for example, may measure comparative IC₅₀ values or percentage inhibition values at a set concentration. Typical methods are described below.

To test the selectivity of the compounds against a panel of PDE's, lysate derived from transiently transfected HEK293 cells (transfected with PDE5A, PDE1B, PDE2A or PDE4A for 48 h) is used as the enzyme source.

The dose response of compounds on PDE5A lysate is performed using the cGMP bulk htrf kit from Cisbio (catnr 62GM2PEC). The principle of this kit is based on the HTRF®D technology (Homogeneous Time-Resolved Fluorescence). The method is based on the competition between native cGMP and the cGMP labeled with d2. The tracer binding is visualized by a monoclonal antibody against cGMP, labeled with Cryptate. The specific signal (i.e. energy transfer) is inversely proportional to the concentration of cGMP in the sample. PDE5A hydrolyses cGMP into 5′GMP; the decrease in cGMP concentration upon PDE5A activity will result in an increased signal. A PDE5A inhibitor will cause a decrease of this signal.

For the enzymatic reaction, a mix of 20 μL with PDE5A lysate, 400 nM cGMP, and the compound is made in a black 384-plate. The reaction buffer consists of Tris 20 mM pH 7.4, 3 mM MgCl₂, 1.5 mM CaCl₂, 0.2 mg/mL BSA and 0.001% Brij-35. After an incubation of 25 minutes at room temperature, the reaction is stopped by the addition of 10 μL labeled cGMP-d2 and 10 μL anti-cGMP-Cryptate. After 1 hour incubation at room temperature, the readout is performed on the Envision (excitation 360 nm; emission donor 615 nm; emission acceptor 665 nm).

All compounds are tested in duplicate starting from 20 μM and 20 nM followed by a ⅓ serial dilution, 8 points (20 μM-6.67 μM-2.22 μM-740 nM-247 nM-82 nM-27 nM-9 nM and 20 nM-6.67 nM-2.22 nM-740 μM-247 μM-82 μM-27 μM-9 μM) in a final concentration of 1% DMSO. As positive control the compound Vardenafil is also added in dose response. For the calculation of z′ and PIN values 1% DMSO is used as positive control (100% inhibition) and lysate in 1% DMSO as negative control (0% inhibition).

For the single dose screening on PDE1B, PDE2A and PDE4A lysates an assay using the cAMP dynamic 2 bulk htrf kit from Cisbio (catnr 62AM4PEC) is used. The principle of this kit is based on the HTRF® technology (Homogeneous Time-Resolved Fluorescence). The method is based on the competition between native cAMP and the cAMP labeled with d2. The tracer binding is visualized by a monoclonal antibody against cAMP, labeled with Cryptate. The specific signal (i.e. energy transfer) is inversely proportional to the concentration of cAMP in the sample.

For the enzymatic reaction, a mixture is made of 10 μL with PDE1B, PDE2A or PDE4A lysate, 100 nM cAMP and the compound (50 nM in a final concentration of 1% DMSO) in a black 384-plate. The reaction buffer is Tris 20 mM pH 7.4, 37.5 U/ml calmodulin, 3 mM MgCl₂, 1.5 mM CaCl₂, 0.2 mg/mL BSA and 0.001% Brij-35®. After an incubation of 25 minutes at room temperature, the reaction is stopped by the addition of 5 μL labelled cAMP-d2 and 5 μL anti-cAMP-Cryptate. After 1 hour incubation at room temperature, the readout is performed on the Envision (excitation 360 nm; emission donor 615 nm; emission acceptor 665 nm).

PDE1B, PDE2A and PDE4A hydrolyse cAMP into 5′AMP; this decrease in cAMP concentration will result in an increase in signal. A PDE1A, PDE2A or PDE4A inhibitor will result in a decrease of this signal.

1% DMSO (100% inhibition) may be used as a positive control, lysate with 1% DMSO (0% inhibition) may be used as a negative control. The positive and negative control are used to calculate z′ and PIN values.

All compounds may be screened at a single concentration of 50 nM. The hit criteria is set at PIN 50 (50% inhibition).

It should be understood that factors such as the differential cell penetration capacity of the various compounds can contribute to discrepancies between the activity of the compounds in the in vitro biochemical and cellular assays.

It will be appreciated by those skilled in the art that the foregoing description is exemplary and explanatory in nature, and is intended to illustrate the invention and its particular embodiments. Through routine experimentation, an artisan will recognise apparent modifications and variations that may be made without departing from the spirit of the invention. Thus, the invention is intended to be defined not by the above description, but by the following claims and their equivalents.

2.4 Development of a High-Throughput Screening Method for the Detection of Endogenous Collagen Type II, Alpha-1 (col2 α1)

Principle of the Assay:

The mouse embryonic cell line ATDC5 is a cell line that can be induced to a chondrogenic fate by certain culturing conditions such as high cell density or certain growth factors. Anabolic compounds can be tested in this cell line for their capacity to induce or enhance the chondrogenic differentiation by measuring a typical chondrocyte markers such as collagen type II, alpha-1 (col2α1), a major constituent of normal cartilage. ATDC5 cells are seeded in 384 well plates and 3 days after plating treated with compounds. Col2α1 deposition is determined 14 days after the start of the infection.

### Control Compounds

FK506 was described to induce chondrogenic differentiation in ATDC5 cells and increase collagen II production (Nishigaki et al, 2002, Eur J. Pharmacol. FK506 induces chondrogenic differentiation of clonal mouse embryonic carcinoma cells, ATDC5).

### Assay Description

ATDC5 cells are seeded on day 0 at 1000 cells/well in 50 μL of DMEM/F12 (InVitrogen), containing 5% heat-inactivated fetal calf serum, 1% Pen/Strep in a 384-well plate previously coated with a 0.1% solution of gelatin in PBS. After 3 days the medium is replaced by the same medium containing 12.5% cell differentiation medium (CDM, Cell applications) and either 0.1% DMSO or a compound solution in DMSO such that DMSO levels are 0.1%. At day 7 medium and compound solutions are refreshed, and at day 10 60 μg/mL 2-Phospho-L-ascorbic acid is added.

Up-regulation of Col2a1 is read at day 13: The medium is removed with a VacuSafe; 50 μL ice-cold MeOH is added and removed immediately by inverting the plate; 50 μL of ice-cold MeOH is added to fix the cells, and plates are incubated for 20 min at −20° C.; MeOH is removed and plates are air-dried for 20 min, followed by 2× washing with 80 μl of phosphate buffered saline (PBS); 75 μL of blocking buffer (0.1% casein in PBS) is added and plates are incubated for at least 2 h at room temperature (RT). After the incubation the blocking buffer is removed; cells are washed with 25 μL of EC buffer (20 mM sodium phosphate, 2 mM EDTA, 400 mM NaCl, 0.2% BSA, 0.05% CHAPS, 0.4% casein, 0.05% NaN₃, pH 7) and 35 μL of the primary antibody (Collagen II Ab-2, Neomarkers MS-235-P) diluted 1/400 in buffer C (20 mM sodium phosphate, 2 mM EDTA, 400 mM NaCl, 1% BSA, pH 7) is added; plates are incubated overnight at 4° C. After the incubation the primary antibody is removed; cells are washed once with 80 μL of PBST (0.5% Tween 20 in PBS) and once with 80 μL1 PBS; 35 μL of the secondary antibody (Goat-anti-mouse Immunoglobulins/HRP. DAKO, PO477; diluted 1/2000 in buffer C) is added; plates are incubated at RT for at least 45 min but no longer than 1 h. After this incubation, the secondary antibody is removed and cells are washed twice with 80 μL PBST and once with 80 μL PBS; 50 μL of luminol substrate is added and after 5 minutes read-out is determined on a luminometer.

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. All such modifications coming within the scope of the appended claims are intended to be included therein.

The chemical names of compounds given in this application were generated using various commercially available chemical naming software tools including MDL's ISIS Draw Autonom Software tool, and were not verified. Particularly, in the event of inconsistency, the depicted structure governs.

### REFERENCES

Andreakos E, et al. (2003). Arthritis Rheum. 48: 1901-12.
Choy E H, Panayi G S. (2001). N Engl J. Med. 344: 907-16.
Clegg D O et al. (2006) N Engl J. Med. 2006.354:795-808. Glucosamine, chondroitin sulfate, and the two in combination for painful knee osteoarthritis.
Coussens L M, et al. (2002). Science 295: 2387-92.
Creemers E E, et al. (2001). Circ Res. 2001 89:201-10
Cunnane G, et al. (2001). Arthritis Rheum 44: 2263-74.
Edwards J. C. W., Szczepanski L., Szechinski J., Filipowicz-Sosnowska A., Emery P., Close D. R.,
Stevens R. M., Shaw T. (2004) N Engl J. Med. 350:2572-2581.
EMBO J. 23:4780-91.
Firestein G S. (2003). Nature. 423:356-61.
Gapski R, et al. (2004). J Periodontol. 75:441-52.
Geng Y, Zhou L, Thompson W J, Lotz M. (1998) Cyclic GMP and cGMP-binding phosphodiesterase are required for interleukin-1-induced nitric oxide synthesis in human articular chondrocytes. J Biol. Chem. 273:27484-91.
Gomez-Reino J J, et al. (2003). Arthritis Rheum. 48: 2122-7.
Goraya T A, Cooper D M. (2005) Ca2+-calmodulin-dependent phosphodiesterase (PDE1): current perspectives. Cell Signal. 17:789-97.
Harder K. W. et al. Biochem. J. 1994 398 395
Kakkar R, Raju R V, Sharma R K. (1999) Calmodulin-dependent cyclic nucleotide phosphodiesterase (PDE1). Cell Mol Life Sci. 55:1164-86.
Kremer J. M., Westhovens R., Leon M., Di Giorgio E., Alten R., Steinfeld S., Russell A., Dougados M., Emery P., Nuamah I. F., Williams G. R., Becker J.-C., Hagerty D. T., Moreland L. W. (2003) N Engl J. Med. 349:1907-1915.
Lee D M, Weinblatt M E (2001). Lancet. 358: 903-11.
Lugnier C. (2006) Pharmacol Ther. 2006 109:366-98. Review. Cyclic nucleotide phosphodiesterase (PDE) superfamily: a new target for the development of specific therapeutic agents.
Murray F et al. (2006) Am J Physiol Lung Cell Mol. Physiol. Expression and Activity of cAMP Phosphodiesterase Isoforms in Pulmonary Artery Smooth Muscle Cells from Patients with Pulmonary Hypertension: Role for PDE1. In publication.
New L, Jiang Y, Han J. (2003) Regulation of PRAK subcellular location by p38 MAP kinases. Mol Biol Cell. 14(6):2603-16.
O'Dell J R, Leff R, Paulsen G, Haire C, Mallek J, Eckhoff P J, Fernandez A, Blakely K, Wees S, Stoner J, Hadley S, Felt J, Palmer W, Waytz P, Churchill M, Klassen L, Moore G. (2002) Arthritis Rheum. 46:1164-70.
O'Dell J R. (2004) Therapeutic strategies for rheumatoid arthritis. N Engl J. Med. 350(25):2591-602.
Reed T M et al. (2002) Phosphodiesterase 1B knock-out mice exhibit exaggerated locomotor hyperactivity and DARPP-32 phosphorylation in response to dopamine agonists and display impaired spatial learning. J. Neurosci. 22:5188-97.
Reif S, Somech R, Brazovski E, Reich R, Belson A, Konikoff F M, Kessler A. (2005) Digestion. 71:124-130.
Rosenberg G A. (2002). Glia. 39:279-91.
S. A. Beers et al. Bioorg. Med. Chem. 1997 5 2203
Schanstra J P, et al. (2002). J Clin Invest. 110:371-9.
Seternes O M, Mikalsen T, Johansen B, Michaelsen E, Armstrong C G, Morrice N A, Turgeon B, Meloche S, Moens U, Keyse S M. (2004) Activation of MK5/PRAK by the atypical MAP kinase ERK3 defines a novel signal transduction pathway.
Shi Y, Kotlyarov A, Laabeta K, Gruber A D, Butt E, Marcus K, Meyer H E, Friedrich A, Volk H D, Gaestel M. (2003) Elimination of protein kinase MK5/PRAK activity by targeted homologous recombination. Mol Cell Biol. 23:7732-41.
Smolen J S, Steiner G. (2003). Nat Rev Drug Discov. 2: 473-88.
St Clair E W, van der Heijde D M, Smolen J S, Maini R N, Bathon J M, Emery P, Keystone E, Schiff M, Kalden J R, Wang B, Dewoody K, Weiss R, Baker D; (2004) Combination of infliximab and methotrexate therapy for early rheumatoid arthritis: a randomized, controlled trial. Arthritis Rheum. 50:3432-43.
Suzuki R, et al. (2004). Treat Respir Med. 3:17-27.
Tenor H, Hedbom E, Hauselmann H J, Schudt C, Hatzelmann A. Phosphodiesterase isoenzyme families in human osteoarthritis chondrocytes—functional importance of phosphodiesterase 4. Br J. Pharmacol. (2002) 135:609-18.
Wieland H A, Michaelis M, Kirschbaum B J, Rudolphi K A. (2005). Nat Rev Drug Discov. 4:331-44. Osteoarthritis—an untreatable disease?
Zhang K Y et al. (2004) A glutamine switch mechanism for nucleotide selectivity by phosphodiesterases. Mol. Cell. 2004 15:279-86.

1. A compound according to formula Ia, Ib, or Ic: 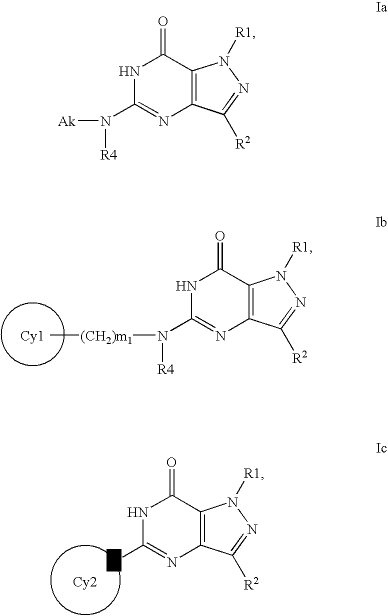
wherein:
Ak is C₁-C₆ alkyl;
Cy1 is substituted or unsubstituted aryl or substituted or unsubstituted heteroaryl;
Cy2 is heterocycloalkyl unsubstituted or substituted with C₁-C₆ alkyl, aralkyl, halo substituted aralkyl, heteroarylalkyl or halo substituted heteroarylalkyl; or
Cy2 is 1,2,3,4-tetrahydroisoquinoline;
R¹ is H, C₁-C₆ alkyl, C₂-C₆ alkenyl, C₂-C₆ alkynyl, cycloalkyl optionally substituted with one or more of OH, CF₃, CN, halogen, —CONH₂, or C₁-C₆ alkyl;
R² is H, C₁-C₆ alkyl, C₂-C₆ alkenyl, C₂-C₆ alkynyl, cycloalkyl optionally substituted with one or more of OH, CF₃, CN, halogen, —CONH₂, or C₁-C₆ alkyl;
R⁴ is H, or alkyl; and
m1 is 0, 1, or 2; provided that when Cy1 is heteroaryl, and the heteroaryl group is joined to —(CH₂)m1- via a N atom of the heteroaryl groups then m1 is 2;
or a pharmaceutically acceptable salt thereof or isotopic variants thereof, stereoisomers or tautomers thereof.

2. The compound according to claim 1 of formula Ia: 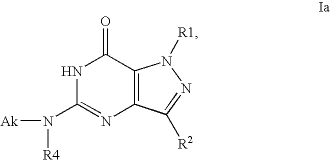
wherein Ak, R¹, R² and R⁴ are as in claim 1.

3. The compound according to claim 1 of formula Ib: 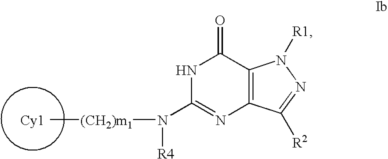
wherein Cy1, R¹, R², R⁴ and ml are as in claim 1.

4. (canceled)

5. The compound according to claim 1 wherein R¹ is H or C₁-C₆ alkyl.

6. The compound according to claim 1 wherein R¹ is Me.

7. The compound according to claim 1 wherein R² is C₁-C₆ alkyl or cycloalkyl.

8. The compound according to claim 1 wherein R² is Me, Et, n-Pr, t-Bu, cyclopropyl, cyclohexyl, or cyclopentyl.

9. The compound according to claim 1 wherein R⁴ is C₁-C₆ alkyl.

10. (canceled)

11. The compound according to claim 1 of formula II: 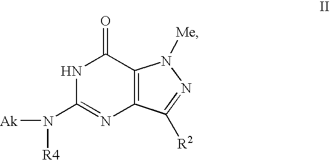
wherein R² is C₁-C₆ alkyl or C₃-C₈ cycloalkyl and R⁴ is C₁-C₆ alkyl.

12. (canceled)

13. (canceled)

14. (canceled)

15. (canceled)

16. The compound according to claim 3 wherein Cy1 is aryl unsubstituted or substituted with C₁-C₆ alkyl halo, halo C₁-C₆ alkyl, C₁-C₆ alkoxy, C₃-C₈ cycloalkyl, heterocycloalkyl, aryl or heteroaryl.

17. The compound according to claim 3 of formulae IIIa or IIIb: 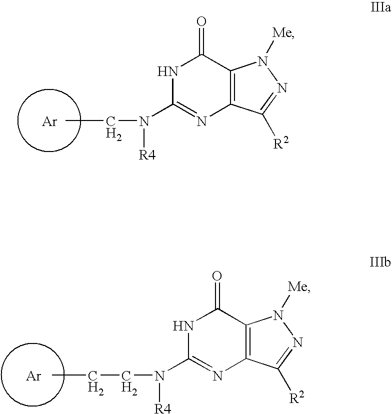
wherein:
Ar is substituted or unsubstituted phenyl;
R² is C₁-C₆ alkyl or C₃-C₈ cycloalkyl; and
R⁴ is C₁-C₆ alkyl.

18. (canceled)

19. The compound according to claim 17 wherein Ar is phenyl substituted with C₃-C₈ cycloalkyl, heterocycloalkyl, aryl or heteroaryl.

20. (canceled)

21. (canceled)

22. (canceled)

23. The compound according to claim 17 of formula IVb: 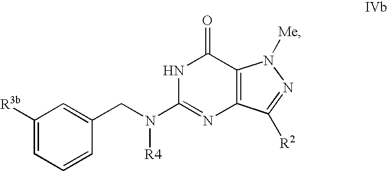
wherein:
R² and R⁴ are as in claim 17; and
R^{3b} is aryl unsubstituted or substituted with C₁-C₆ alkyl, halo, halo C₁-C₆ alkyl, C₁-C₆ alkoxy, C₃-C₈ cycloalkyl, heterocycloalkyl, aryl or heteroaryl; heterocycloalkyl or heteroaryl.

24. The compound according to claim 23 wherein R^{3b} is selected from substituted or unsubstituted piperidinyl, piperazinyl, morpholinyl, pyrrolyl, pyrazolyl, imidazolyl, triazolyl, tetrazolyl, thiazolyl, oxazolyl, pyridyl, and pyrimidinyl.

25. (canceled)

26. (canceled)

27. The compound according to claim 17 of formulae Va, Vb, Vc, Vd, Ve, Vf, Vg, or Vh: 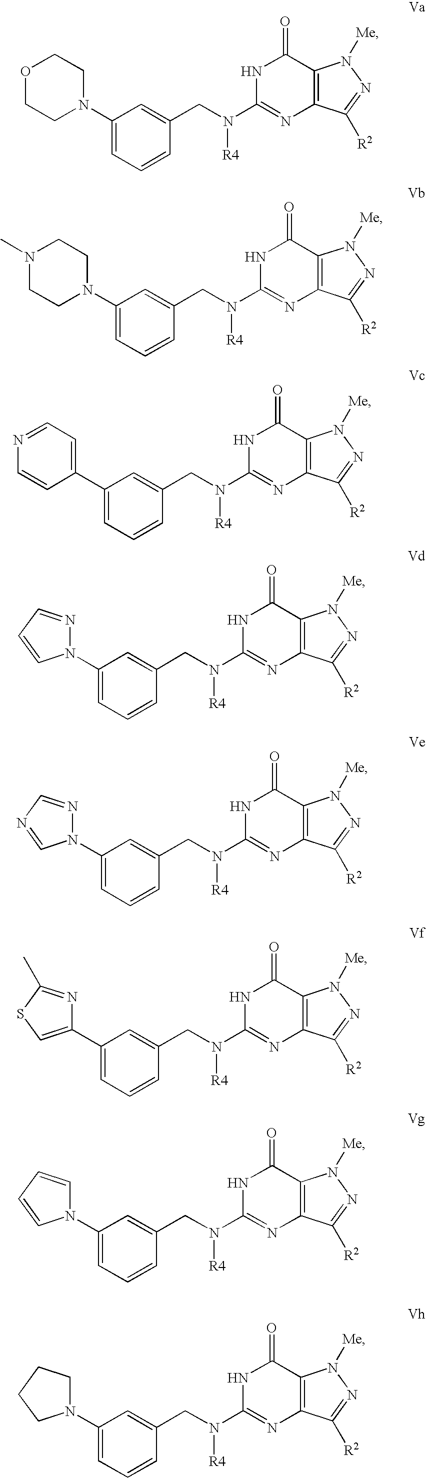
wherein R² and R⁴ are as in claim 17.

28. (canceled)

29. The compound according to claim 3 of formulae VIa or VIb: 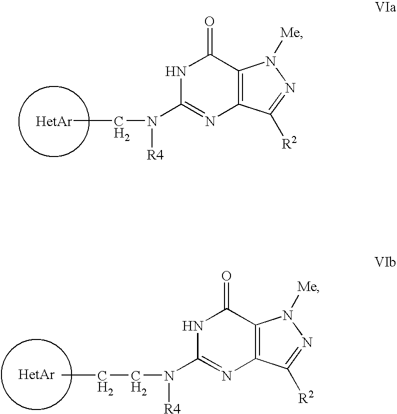
wherein:
HetAr is heteroaryl unsubstituted or substituted with C₁-C₆ alkyl, halo, halo C₁-C₆ alkyl, C₁-C₆ alkoxy C₃-C₈ cycloalkyl, heterocycloalkyl, aryl or heteroaryl;
R² is C₁-C₆ alkyl or cycloalkyl;
R⁴ is C₁-C₆ alkyl.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. The compound according to claim 29 wherein HetAr is selected from substituted or unsubstituted pyrrolyl, pyrazolyl, imidazolyl, triazolyl, tetrazolyl, thiazolyl, isoxazolyl, and oxazolyl.

42. The compound according to claim 29 wherein HetAr is selected from pyrrolyl, pyrazolyl, imidazolyl, triazolyl, tetrazolyl, thiazolyl, isoxazolyl, and oxazolyl; substituted with one or more groups selected from C₁-C₆ alkyl, halo, haloalkyl, and phenyl.

43. (canceled)

44. (canceled)

45. The compound according to claim 29 of formulae XIa, XIb, XIc, XId, or XIe: 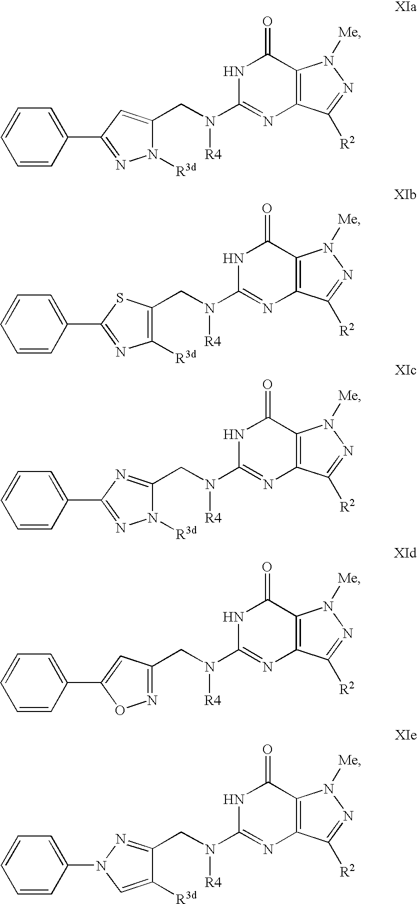
wherein R² and R⁴ are as in claim 29; and R^{3d} is H, C₁-C₆ alkyl or C₁-C₆ haloalkyl.

46. (canceled)

47. (canceled)

48. (canceled)

49. (canceled)

50. (canceled)

51. (canceled)

52. (canceled)

53. (canceled)

54. A compound according to claim 1 wherein the compound is selected from
5-(4-(2-chloro-6-fluorobenzyl)-1,4-diazepan-1-yl)-1-methyl-3-propyl-1H-pyrazolo[4,3-d]pyrimidin-7(6H)-one;
5-((3,4-dichlorobenzyl)(methyl)amino)-1-methyl-3-propyl-1H-pyrazolo[4,3-d]pyrimidin-7(6H)-one;
1-methyl-5-(methyl(4-(piperidin-1-yl)benzyl)amino)-3-propyl-1H-pyrazolo[4,3-d]pyrimidin-7(6H)-one;
5-(dipropylamino)-1-methyl-3-propyl-1H-pyrazolo[4,3-d]pyrimidin-7(6H)-one;
1-methyl-5-(methyl(3-(trifluoromethyl)benzyl)amino)-3-propyl-1H-pyrazolo[4,3-d]pyrimidin-7(6H)-one;
1-methyl-5-(4-(4-methylpiperazin-1-yl)benzylamino)-3-propyl-1H-pyrazolo[4,3-d]pyrimidin-7(6H)-one;
5-(1H-indazol-5-ylamino)-1-methyl-3-propyl-1H-pyrazolo[4,3-d]pyrimidin-7(6H)-one;
5-(3,4-dihydroisoquinolin-2(1H)-yl)-1-methyl-3-propyl-1H-pyrazolo[4,3-d]pyrimidin-7(6H)-one;
5-((4-methoxybenzyl)(methyl)amino)-1-methyl-3-propyl-1H-pyrazolo[4,3-d]pyrimidin-7(6H)-one;
1-methyl-5-(methyl(3-morpholinobenzyl)amino)-3-propyl-1H-pyrazolo[4,3-d]pyrimidin-7(6H)-one;
1-methyl-5-(3-(piperidin-1-yl)benzylamino)-3-propyl-1H-pyrazolo[4,3-d]pyrimidin-7(6H)-one;
1-methyl-5-(methyl(3-(pyrimidin-5-yl)benzyl)amino)-3-propyl-1H-pyrazolo[4,3-d]pyrimidin-7(6H)-one;
1-methyl-5-(methyl(4-(pyrimidin-5-yl)benzyl)amino)-3-propyl-1H-pyrazolo[4,3-d]pyrimidin-7(6H)-one;
5-(((1,5-dimethyl-1H-pyrazol-3-yl)methyl)(methyl)amino)-1-methyl-3-propyl-1H-pyrazolo[4,3-d]pyrimidin-7(6H)-one;
5-((3-chlorobenzyl)(methyl)amino)-1-methyl-3-propyl-1H-pyrazolo[4,3-d]pyrimidin-7(6H)-one;
1-methyl-5-(methyl(4-(trifluoromethyl)benzyl)amino)-3-propyl-1H-pyrazolo[4,3-d]pyrimidin-7(6H)-one;
5-((4-chlorobenzyl)(methyl)amino)-1-methyl-3-propyl-1H-pyrazolo[4,3-d]pyrimidin-7(6H)-one;
5-(3-fluorobenzylamino)-1-methyl-3-propyl-1H-pyrazolo[4,3-d]pyrimidin-7(6H)-one;
5-(isopentylamino)-1-methyl-3-propyl-1H-pyrazolo[4,3-d]pyrimidin-7(6H)-one;
5-((3,4-dimethoxyphenethyl)(methyl)amino)-1-methyl-3-propyl-1H-pyrazolo[4,3-d]pyrimidin-7(6H)-one;
5-((4-fluorobenzyl)(methyl)amino)-1-methyl-3-propyl-1H-pyrazolo[4,3-d]pyrimidin-7(6H)-one;
1-methyl-5-(methyl(phenethyl)amino)-3-propyl-1H-pyrazolo[4,3-d]pyrimidin-7(6H)-one;
5-(4-fluorobenzylamino)-1-methyl-3-propyl-1H-pyrazolo[4,3-d]pyrimidin-7(6H)-one;
5-((2-chlorobenzyl)(methyl)amino)-1-methyl-3-propyl-1H-pyrazolo[4,3-d]pyrimidin-7(6H)-one;
1-methyl-5-(methyl((5-phenylisoxazol-3-yl)methyl)amino)-3-propyl-1H-pyrazolo[4,3-d]pyrimidin-7(6H)-one;
1-methyl-3-propyl-5-(3-(pyrrolidin-1-yl)benzylamino)-1H-pyrazolo[4,3-d]pyrimidin-7(6H)-one;
1-methyl-5-(4-(morpholinomethyl)benzylamino)-3-propyl-1H-pyrazolo[4,3-d]pyrimidin-7(6H)-one;
5-(benzyl(isopropyl)amino)-1-methyl-3-propyl-1H-pyrazolo[4,3-d]pyrimidin-7(6H)-one;
5-(diisobutylamino)-1-methyl-3-propyl-1H-pyrazolo[4,3-d]pyrimidin-7(6H)-one;
1-methyl-5-(methyl(propyl)amino)-3-propyl-1H-pyrazolo[4,3-d]pyrimidin-7(6H)-one;
5-(3,5-bis(trifluoromethyl)benzylamino)-1-methyl-3-propyl-1H-pyrazolo[4,3-d]pyrimidin-7(6H)-one;
5-(3,4-dichlorobenzylamino)-1-methyl-3-propyl-1H-pyrazolo[4,3-d]pyrimidin-7(6H)-one;
5-(benzyl(methyl)amino)-1-methyl-3-propyl-1H-pyrazolo[4,3-d]pyrimidin-7(6H)-one;
5-(6-fluoro-3,4-dihydroisoquinolin-2(1H)-yl)-1-methyl-3-propyl-1H-pyrazolo[4,3-d]pyrimidin-7(6H)-one;
1-methyl-5-(methyl((1-methyl-1H-indazol-3-yl)methyl)amino)-3-propyl-1H-pyrazolo[4,3-d]pyrimidin-7(6H)-one;
1-methyl-5-(methyl(thieno[2,3-b]pyridin-2-ylmethyl)amino)-3-propyl-1H-pyrazolo[4,3-d]pyrimidin-7(6H)-one;
5-(6,7-dimethoxy-1-methyl-3,4-dihydroisoquinolin-2(1H)-yl)-1-methyl-3-propyl-1H-pyrazolo[4,3-d]pyrimidin-7(6H)-one;
1-methyl-5-(methyl((1-methyl-3-phenyl-1H-pyrazol-5-yl)methyl)amino)-3-propyl-1H-pyrazolo[4,3-d]pyrimidin-7(6H)-one;
1-methyl-5-(methyl((2-(4-(trifluoromethyl)phenyl)thiazol-4-yl)methyl)amino)-3-propyl-1H-pyrazolo[4,3-d]pyrimidin-7(6H)-one;
1-methyl-5-(methyl(4-(pyridin-4-yl)benzyl)amino)-3-propyl-1H-pyrazolo[4,3-d]pyrimidin-7(6H)-one;
9,
1-methyl-5-(methyl((2-(pyrrolidin-1-yl)pyridin-4-yl)methyl)amino)-3-propyl-1H-pyrazolo[4,3-d]pyrimidin-7(6H)-one;
1-methyl-5-(methyl((6-morpholinopyridin-2-yl)methyl)amino)-3-propyl-1H-pyrazolo[4,3-d]pyrimidin-7(6H)-one;
1-methyl-5-(methyl(3-(pyridin-4-yl)benzyl)amino)-3-propyl-1H-pyrazolo[4,3-d]pyrimidin-7(6H)-one;
1-methyl-5-(methyl(4-(pyridin-2-yl)benzyl)amino)-3-propyl-1H-pyrazolo[4,3-d]pyrimidin-7(6H)-one;
5-((3,4-dichlorobenzyl)(isopentyl)amino)-1-methyl-3-propyl-1H-pyrazolo[4,3-d]pyrimidin-7(6H)-one;
5-(butyl(3,4-dichlorobenzyl)amino)-1-methyl-3-propyl-1H-pyrazolo[4,3-d]pyrimidin-7(6H)-one;
1-methyl-5-(methyl((4-methyl-2-phenylthiazol-5-yl)methyl)amino)-3-propyl-1H-pyrazolo[4,3-d]pyrimidin-7(6H)-one;
1-methyl-5-(methyl(3-(2-morpholinoethoxy)benzyl)amino)-3-propyl-1H-pyrazolo[4,3-d]pyrimidin-7(6H)-one;
1-methyl-5-(methyl(4-(2-morpholinoethoxy)benzyl)amino)-3-propyl-1H-pyrazolo[4,3-d]pyrimidin-7(6H)-one;
1-methyl-5-(2-(2-morpholinoethoxy)benzylamino)-3-propyl-1H-pyrazolo[4,3-d]pyrimidin-7(6H)-one;
1-methyl-5-((5-methyl-3-phenylisoxazol-4-yl)methylamino)-3-propyl-1H-pyrazolo[4,3-d]pyrimidin-7(6H)-one;
1-methyl-5-((5-methyl-2-phenyl-2H-1,2,3-triazol-4-yl)methylamino)-3-propyl-1H-pyrazolo[4,3-d]pyrimidin-7(6H)-one;
1-methyl-5-(3-(morpholinomethyl)benzylamino)-3-propyl-1H-pyrazolo[4,3-d]pyrimidin-7(6H)-one;
1-methyl-5-((2-morpholinopyridin-4-yl)methylamino)-3-propyl-1H-pyrazolo[4,3-d]pyrimidin-7(6H)-one;
1-methyl-5-(3-(2-morpholinoethoxy)benzylamino)-3-propyl-1H-pyrazolo[4,3-d]pyrimidin-7(6H)-one;
1-methyl-5-(3-morpholinobenzylamino)-3-propyl-1H-pyrazolo[4,3-d]pyrimidin-7(6H)-one;
1-methyl-5-((1-phenyl-1H-pyrazol-3-yl)methylamino)-3-propyl-1H-pyrazolo[4,3-d]pyrimidin-7(6H)-one;
1-methyl-5-(methyl(naphthalen-1-ylmethyl)amino)-3-propyl-1H-pyrazolo[4,3-d]pyrimidin-7(6H)-one;
5-(4-(2,2-diphenylacetoyl)piperazin-1-yl)-1-methyl-3-propyl-1H-pyrazolo[4,3-d]pyrimidin-7(6H)-one;
1-methyl-3-propyl-5-(4-(trifluoromethyl)piperidin-1-yl)-1H-pyrazolo[4,3-d]pyrimidin-7(6H)-one;
1-methyl-5-(methyl(4-morpholinobenzyl)amino)-3-propyl-1H-pyrazolo[4,3-d]pyrimidin-7(6H)-one;
5-((2,3-dihydrobenzofuran-5-yl)methylamino)-1-methyl-3-propyl-1H-pyrazolo[4,3-d]pyrimidin-7(6H)-one;
5-(1H-pyrazol-1-yl)benzylamino)-1-methyl-3-propyl-1H-pyrazolo[4,3-d]pyrimidin-7(6H)-one;
5-(1H-pyrrol-1-yl)benzylamino)-1-methyl-3-propyl-1H-pyrazolo[4,3-d]pyrimidin-7(6H)-one;
1-methyl-5-(methyl(3-(2-methylthiazol-4-yl)benzyl)amino)-3-propyl-1H-pyrazolo[4,3-d]pyrimidin-7(6H)-one;
1-methyl-5-((1-methyl-3-phenyl-1H-pyrazol-5-yl)methylamino)-3-propyl-1H-pyrazolo[4,3-d]pyrimidin-7(6H)-one;
1-methyl-5-(methyl-1H-pyrazol-3-yl)benzylamino)-3-propyl-1H-pyrazolo[4,3-d]pyrimidin-7(6H)-one;
1-methyl-5-((1-methyl-1H-indol-5-yl)methylamino)-3-propyl-1H-pyrazolo[4,3-d]pyrimidin-7(6H)-one;
1-methyl-5-((1-methyl-1H-indol-6-yl)methylamino)-3-propyl-1H-pyrazolo[4,3-d]pyrimidin-7(6H)-one;
3-cyclopentyl-5-((4-fluorobenzyl)(methyl)amino)-1-methyl-1H-pyrazolo[4,3-d]pyrimidin-7(6H)-one;
5-((4-chlorobenzyl)(methyl)amino)-3-cyclopentyl-1-methyl-1H-pyrazolo[4,3-d]pyrimidin-7(6H)-one;
1-methyl-5-((4-methyl-2-phenylthiazol-5-yl)methylamino)-3-propyl-1H-pyrazolo[4,3-d]pyrimidin-7(6H)-one;
5-(1H-1,2,4-triazol-1-yl)benzylamino)-1-methyl-3-propyl-1H-pyrazolo[4,3-d]pyrimidin-7(6H)-one;
5-(isobutyl(3-morpholinobenzyl)amino)-1-methyl-3-propyl-1H-pyrazolo[4,3-d]pyrimidin-7(6H)-one;
5-((4-chlorophenethyl)(propyl)amino)-1-methyl-3-propyl-1H-pyrazolo[4,3-d]pyrimidin-7(6H)-one;
3-cyclopentyl-5-(3,4-dichlorobenzylamino)-1-methyl-1H-pyrazolo[4,3-d]pyrimidin-7(6H)-one;
3-cyclopentyl-5-((3,4-dichlorobenzyl)(methyl)amino)-1-methyl-1H-pyrazolo[4,3-d]pyrimidin-7(6H)-one;
3-cyclopentyl-5-(dipropylamino)-1-methyl-1H-pyrazolo[4,3-d]pyrimidin-7(6H)-one;
5-(4-chlorophenethylamino)-1-methyl-3-propyl-1H-pyrazolo[4,3-d]pyrimidin-7(6H)-one;
3-tert-butyl-1-methyl-5-(methyl((4-methyl-2-phenylthiazol-5-yl)methyl)amino)-1H-pyrazolo[4,3-d]pyrimidin-7(6H)-one;
3-tert-butyl-1-methyl-5-(methyl((1-methyl-3-phenyl-1H-pyrazol-5-yl)methyl)amino)-1H-pyrazolo[4,3-d]pyrimidin-7(6H)-one;
5-(3,4-diethoxyphenethylamino)-1-methyl-3-propyl-1H-pyrazolo[4,3-d]pyrimidin-7(6H)-one;
5-([1,2,4]triazolo[4,3-a]pyridin-3-ylmethylamino)-1-methyl-3-propyl-1H-pyrazolo[4,3-d]pyrimidin-7(6H)-one;
3-cyclopentyl-1-methyl-5-(2-methylpiperidin-1-yl)-1H-pyrazolo[4,3-d]pyrimidin-7(6H)-one;
3-cyclopentyl-1-methyl-5-(piperidin-1-yl)-1H-pyrazolo[4,3-d]pyrimidin-7(6H)-one;
3-cyclopentyl-1-methyl-5-(2-phenylpyrrolidin-1-yl)-1H-pyrazolo[4,3-d]pyrimidin-7(6H)-one;
5-(4-fluorophenethylamino)-1-methyl-3-propyl-1H-pyrazolo[4,3-d]pyrimidin-7(6H)-one;
1-methyl-5-(2-(1-phenyl-1H-pyrazol-4-yl)ethylamino)-3-propyl-1H-pyrazolo[4,3-d]pyrimidin-7(6H)-one;
3-tert-butyl-5-(dipropylamino)-1-methyl-1H-pyrazolo[4,3-d]pyrimidin-7(6H)-one;
3-tert-butyl-1-methyl-5-(methyl(3-(2-methylthiazol-4-yl)benzyl)amino)-1H-pyrazolo[4,3-d]pyrimidin-7(6H)-one;
5-((1-benzyl-3,5-dimethyl-1H-pyrazol-4-yl)methylamino)-1-methyl-3-propyl-1H-pyrazolo[4,3-d]pyrimidin-7(6H)-one;
3-tert-butyl-1-methyl-5-(3-(4-methylpiperazin-1-yl)benzylamino)-1H-pyrazolo[4,3-d]pyrimidin-7(6H)-one;
5-((1H-pyrazol-1-yl)benzyl)(isobutyl)amino)-3-tert-butyl-1-methyl-1H-pyrazolo[4,3-d]pyrimidin-7(6H)-one;
5-((1H-pyrazol-1-yl)benzyl)(isobutyl)amino)-1-methyl-3-propyl-1H-pyrazolo[4,3-d]pyrimidin-7(6H)-one;
5-((1H-1,2,4-triazol-1-yl)benzyl)(isobutyl)amino)-3-tert-butyl-1-methyl-1H-pyrazolo[4,3-d]pyrimidin-7(6H)-one;
5-((1H-1,2,4-triazol-1-yl)benzyl)(isobutyl)amino)-1-methyl-3-propyl-1H-pyrazolo[4,3-d]pyrimidin-7(6H)-one;
3-tert-butyl-5-((4-fluorobenzyl)(isobutyl)amino)-1-methyl-1H-pyrazolo[4,3-d]pyrimidin-7(6H)-one;
5-((4-fluorobenzyl)(isobutyl)amino)-1-methyl-3-propyl-1H-pyrazolo[4,3-d]pyrimidin-7(6H)-one;
3-tert-butyl-5-(isobutyl(methyl)amino)-1-methyl-1H-pyrazolo[4,3-d]pyrimidin-7(6H)-one;
5-((1H-pyrrol-1-yl)benzyl)(isobutyl)amino)-3-tert-butyl-1-methyl-1H-pyrazolo[4,3-d]pyrimidin-7(6H)-one;
5-((1H-pyrrol-1-yl)benzyl)(isobutyl)amino)-1-methyl-3-propyl-1H-pyrazolo[4,3-d]pyrimidin-7(6H)-one;
5-(isobutyl(3-(4-methylpiperazin-1-yl)benzyl)amino)-1-methyl-3-propyl-1H-pyrazolo[4,3-d]pyrimidin-7(6H)-one;
3-tert-butyl-5-((3,3-dimethylbutyl)(3-morpholinobenzyl)amino)-1-methyl-1H-pyrazolo[4,3-d]pyrimidin-7(6H)-one;
3-tert-butyl-1-methyl-5-((2-methylpentyl)(3-morpholinobenzyl)amino)-1H-pyrazolo[4,3-d]pyrimidin-7(6H)-one;
3-tert-butyl-5-((2-ethylbutyl)(methyl)amino)-1-methyl-1H-pyrazolo[4,3-d]pyrimidin-7(6H)-one;
5-((1H-pyrazol-1-yl)benzyl)(isopentyl)amino)-1-methyl-3-propyl-1H-pyrazolo[4,3-d]pyrimidin-7(6H)-one;
5-((1H-pyrazol-1-yl)benzyl)(2-ethylbutyl)amino)-1-methyl-3-propyl-1H-pyrazolo[4,3-d]pyrimidin-7(6H)-one;
5-((3,3-dimethylbutyl)(3-morpholinobenzyl)amino)-1-methyl-3-propyl-1H-pyrazolo[4,3-d]pyrimidin-7(6H)-one;
3-tert-butyl-5-((2-ethylbutyl)(3-morpholinobenzyl)amino)-1-methyl-1H-pyrazolo[4,3-d]pyrimidin-7(6H)-one;
5-((3,3-dimethylbutyl)(methyl)amino)-1-methyl-3-propyl-1H-pyrazolo[4,3-d]pyrimidin-7(6H)-one;
3-tert-butyl-5-((3,3-dimethylbutyl)(methyl)amino)-1-methyl-1H-pyrazolo[4,3-d]pyrimidin-7(6H)-one;
1-methyl-5-(methyl(2-methylbutyl)amino)-3-propyl-1H-pyrazolo[4,3-d]pyrimidin-7(6H)-one;
1-methyl-5-((2-methylpentyl)(3-morpholinobenzyl)amino)-3-propyl-1H-pyrazolo[4,3-d]pyrimidin-7(6H)-one;
1-methyl-5-((2-methylbutyl)(3-morpholinobenzyl)amino)-3-propyl-1H-pyrazolo[4,3-d]pyrimidin-7(6H)-one;
3-tert-butyl-1-methyl-5-(methyl(2-methylpentyl)amino)-1H-pyrazolo[4,3-d]pyrimidin-7(6H)-one;
5-((1H-1,2,4-triazol-1-yl)benzyl)(2-methylbutyl)amino)-1-methyl-3-propyl-1H-pyrazolo[4,3-d]pyrimidin-7(6H)-one;
3-tert-butyl-1-methyl-5-((2-methylbutyl)(3-morpholinobenzyl)amino)-1H-pyrazolo[4,3-d]pyrimidin-7(6H)-one;
5-((1H-1,2,4-triazol-1-yl)benzyl)(2-methylbutyl)amino)-3-tert-butyl-1-methyl-1H-pyrazolo[4,3-d]pyrimidin-7(6H)-one;
5-((1H-pyrazol-1-yl)benzyl)(neopentyl)amino)-1-methyl-3-propyl-1H-pyrazolo[4,3-d]pyrimidin-7(6H)-one;
4-((1-methyl-7-oxo-3-propyl-6,7-dihydro-1H-pyrazolo[4,3-d]pyrimidin-5-ylamino)methyl)benzenesulfonamide;
5-((1H-pyrazol-1-yl)benzyl)(2-ethylbutyl)amino)-3-tert-butyl-1-methyl-1H-pyrazolo[4,3-d]pyrimidin-7(6H)-one;
5-((1H-pyrazol-1-yl)benzyl)(2-methylpentyl)amino)-3-tert-butyl-1-methyl-1H-pyrazolo[4,3-d]pyrimidin-7(6H)-one;
1-methyl-5-(methyl(2-methylpentyl)amino)-3-propyl-1H-pyrazolo[4,3-d]pyrimidin-7(6H)-one;
3-tert-butyl-1-ethyl-5-(isobutyl(3-morpholinobenzyl)amino)-1H-pyrazolo[4,3-d]pyrimidin-7(6H)-one; and
3-cyclopropyl-5-(dipropylamino)-1-methyl-1H-pyrazolo[4,3-d]pyrimidin-7(6H)-one.

55. A pharmaceutical composition comprising an effective amount of a compound according to claim 1, in admixture with a pharmaceutically acceptable carrier, diluent or excipient.

56. A method of treating a disease involving degradation of cartilage, comprising administering an effective amount of a compound according to claim 1.

57. The method according to claim 56, wherein the disease is osteoarthritis.

58. A method of treating a disease involving inflammation, comprising administering an effective amount of a compound according to claim 1.

59. (canceled)
